SEQUENCE LISTING (61 sequences)

SEQ ID NO: 1 - DNA, 20 bp, Artificial sequence
  hAURKB-F2 forward primer
aaggagctgcagaagagctg

SEQ ID NO: 2 - DNA, 20 bp, Artificial sequence
  hAURKB-R2 reverse primer
ccttgagccctaagagcaga

SEQ ID NO: 3 - DNA, 20 bp, Artificial sequence
  hCDK1-F1 forward primer
ctggggtcagctcgttactc

SEQ ID NO: 4 - DNA, 20 bp, Artificial sequence
  hCDK1-R1 reverse primer
tctgaatccccatggaaaag

SEQ ID NO: 5 - DNA, 21 bp, Artificial sequence
  hCKS2-F1 forward primer
cactacgagtaccggcatgtt

SEQ ID NO: 6 - DNA, 21 bp, Artificial sequence
  hCKS2-R1 reverse primer
tgttggacaccaagtctcctc

SEQ ID NO: 7 - DNA, 20 bp, Artificial sequence
  hCLSPN-F2 forward primer
aaggagcgaattgaacgaga

SEQ ID NO: 8 - DNA, 20 bp, Artificial sequence
  hCLSPN-R2 reverse primer
tgcagtgctttggctgtaac

SEQ ID NO: 9 - DNA, 20 bp, Artificial sequence
  hDLGAP5-F2 forward primer
cgtccagaccgagtgttctt

SEQ ID NO: 10 - DNA, 20 bp, Artificial sequence
  hDLGAP5-R2 reverse primer
atccttcctgtgtcgactgg

SEQ ID NO: 11 - DNA, 20 bp, Artificial sequence
  hFAM83D-F1 forward primer
cagtggtcatggacgtgttc

SEQ ID NO: 12 - DNA, 20 bp, Artificial sequence
  hFAM83D-R1 reverse primer
caactccctgtttcctgcat

SEQ ID NO: 13 - DNA, 22 bp, Artificial sequence
  hNCAPH-F2 forward primer
ggctcagaacctctccatacct

SEQ ID NO: 14 - DNA, 22 bp, Artificial sequence
  hNCAPH-R2 reverse primer
gaggtcctctgttccttccagt

SEQ ID NO: 15 - DNA, 20 bp, Artificial sequence
  hNUSAP1-F2 forward primer
aagcgctctgctatctctgc

SEQ ID NO: 16 - DNA, 20 bp, Artificial sequence
  hNUSAP1-R2 reverse primer
ttctggctggagtcttggtc

SEQ ID NO: 17 - DNA, 22 bp, Artificial sequence
  hSPC25-F2 forward primer
ttcaaaagtacggacacctcct

SEQ ID NO: 18 - DNA, 22 bp, Artificial sequence
  hSPC25-R2 reverse primer
ctcaaccattcgttcttcttcc

SEQ ID NO: 19 - DNA, 20 bp, Artificial sequence
  hTACC3-F2 forward primer
tttcgccaccagaagttacc

SEQ ID NO: 20 - DNA, 20 bp, Artificial sequence
  hTACC3-R2 reverse primer
tcatagctttggccaggttc

SEQ ID NO: 21 - DNA, 22 bp, Artificial sequence
  hUBE2C-F1 forward primer
acccaacattgatagtcccttg

SEQ ID NO: 22 - DNA, 22 bp, Artificial sequence
  hUBE2C-R1 reverse primer
gctggtgacctgctttgagtag

SEQ ID NO: 23 - DNA, 27 bp, Artificial sequence
  3R1F forward primer
gcagagatcatttcttgacctgtggag

SEQ ID NO: 24 - DNA, 20 bp, Artificial sequence
  3R1R reverse primer
agcccttggccaggaaacaa

SEQ ID NO: 25 - DNA, 22 bp, Artificial sequence
  3LF forward primer
cttcaaatgctaacccgatgac

SEQ ID NO: 26 - DNA, 22 bp, Artificial sequence
  3LR reverse primer
tctttctcgagctatcgaccag

SEQ ID NO: 27 - DNA, 33 bp, Artificial sequence
  BRD3iso2HA- F forward primer
gttccagattacgctatgtccaccgccacgaca

SEQ ID NO: 28 - DNA, 39 bp, Artificial sequence
  BRD3iso2HA- R reverse primer
atcgtatgggtacatagcctgcttttttgtacaaacttg

SEQ ID NO: 29 - DNA, 25 bp, Artificial sequence
  BRD2-F forward primer
atgctgcaaaacgtgactccccaca

SEQ ID NO: 30 - DNA, 27 bp, Artificial sequence
  BRD2-R reverse primer
ttagcctgagtctgaatcactggtgtc

SEQ ID NO: 31 - DNA, 23 bp, Artificial sequence
  3DF forward primer
atgtccaccgccacgacagtcgc

SEQ ID NO: 32 - DNA, 27 bp, Artificial sequence
  3DR reverse primer
tcattctgagtcactgctgtcagagct

SEQ ID NO: 33 - DNA, 20 bp, Artificial sequence
  hpion-1-F forward primer
tctctgcctgccattcattt

SEQ ID NO: 34 - DNA, 20 bp, Artificial sequence
  hpion-1-R reverse primer
gcactgaggaatgtggcaat

SEQ ID NO: 35 - DNA, 20 bp, Artificial sequence
  hCAMKK1-1-F forward primer
gcgtcagcaaccagtttgag

SEQ ID NO: 36 - DNA, 20 bp, Artificial sequence
  hCAMKK1-1-R reverse primer
agtggcccatacatccaagg

SEQ ID NO: 37 - DNA, 24 bp, Artificial sequence
  hm-GAPDH-hao-F forward primer
ccttcattgacctcaactacatgg

SEQ ID NO: 38 - DNA, 24 bp, Artificial sequence
  hm-GAPDH-hao-R reverse primer
tcgctcctggaagatggtgatggg

SEQ ID NO: 39 - DNA, 28 bp, Artificial sequence
  attR1-F forward primer
caacaagtttgtacaaaaaagctgaacg

SEQ ID NO: 40 - DNA, 44 bp, Artificial sequence
  attrR2-stop-R reverse primer
tcaactagttactaaaccactttgtacaagaaagctgaacgaga

SEQ ID NO: 41 - DNA, 27 bp, Artificial sequence
  3R2R reverse primer
tcaaactccacaggtcaagaaatgatc

SEQ ID NO: 42 - DNA, 62 bp, Artificial sequence
  BRD3S-sh3sn forward primer for cloning BRD3R shRNA
ctaggaacctctgtaattgtttcctggctcgagccaggaaacaattacagaggttctttttt

SEQ ID NO: 43 - DNA, 58 bp, Artificial sequence
  BRD3S-sh3as reverse primer for cloning BRD3R shRNA
aaaaaagaacctctgtaattgtttcctggctcgagccaggaaacaattacagaggttc

SEQ ID NO: 44 - DNA, 5673 bp, Homo sapiens
  misc_feature (1)..(5673)
tgccggggccggcgagccaaagaggagccggccgcgcgggccgggaggggacggccgccggagccgcgaggccaactgtcgcctggttgggcccggaaatgggacgtcgcgctttctcagggagcgtagaagcagccagggcctctccaagccgctgctgtgacagaaagtgagtgagctgccggaggatgtccaccgccacgacagtcgcccccgcggggatcccggcgaccccgggccctgtgaacccaccccccccggaggtctccaaccccagcaagcccggccgcaagaccaaccagctgcagtacatgcagaatgtggtggtgaagacgctctggaaacaccagttcgcctggcccttctaccagcccgtggacgcaatcaaattgaacctgccggattatcataaaataattaaaaacccaatggatatggggactattaagaagagactagaaaataattattattggagtgcaagcgaatgtatgcaggacttcaacaccatgtttacaaattgttacatttataacaagcccacagatgacatagtgctaatggcccaagctttagagaaaatttttctacaaaaagtggcccagatgccccaagaggaagttgaattattaccccctgctccaaagggcaaaggtcggaagccggctgcgggagcccagagcgcaggtacacagcaagtggcggccgtgtcctctgtctccccagcgaccccctttcagagcgtgccccccaccgtctcccagacgcccgtcatcgctgccacccctgtaccaaccatcactgcaaacgtcacgtcggtcccagtccccccagctgccgccccacctcctcctgccacacccatcgtccccgtggtccctcctacgccgcctgtcgtcaagaaaaagggcgtgaagcggaaagcagacacaaccactcccacgacgtcggccatcactgccagccggagtgagtcgcccccgccgttgtcagaccccaagcaggccaaagtggtggcccggcgggagagtggtggccgccccatcaagcctcccaagaaggacctggaggacggcgaggtgccccagcacgcaggcaagaagggcaagctgtcggagcacctacgctactgcgacagcatcctcagggagatgctatccaagaagcacgcggcctacgcctggcccttctacaagccagtggatgccgaggccctggagctgcacgactaccacgacatcatcaagcacccgatggacctcagcaccgtgaaaaggaagatggatggccgagagtacccagacgcacagggctttgctgctgatgtccggctgatgttctcgaattgctacaaatacaatcccccagaccacgaggttgtggccatggcccggaagctccaggacgtgtttgagatgaggtttgccaagatgccagatgagcccgtggaggcaccggcgctgcctgcccccgcggcccccatggtgagcaagggcgctgagagcagccgtagcagtgaggagagctcttcggactcaggcagctcggactcggaggaggagcgggccaccaggctggcggagctgcaggagcagctgaaggccgtgcacgagcagctggccgccctgtctcaggccccagtaaacaaaccaaagaagaagaaggagaagaaggagaaggagaagaagaagaaggacaaggagaaggagaaggagaagcacaaagtgaaggccgaggaagagaagaaggccaaggtggctccgcctgccaagcaggctcagcagaagaaggctcctgccaagaaggccaacagcacgaccacggccggcagacagctgaagaaaggcggcaagcaggcatctgcctcctacgactcagaggaagaggaggagggcctgcccatgagctacgatgaaaagcgccagcttagcctggacatcaaccggctgcccggggagaagctgggccgggtagtgcacatcatccaatctcgggagccctcgctcagggactccaaccccgacgagatagaaattgactttgagactctgaaacccaccactttgcgggaactggagagatatgtcaagtcttgtttacagaaaaagcaaaggaaaccgttctcagcaagcgggaagaaacaggcagccaagtcgaaagaggagctagctcaggaaaagaagaaggagctggaaaagcgtctgcaggatgtcagcgggcagctgagcagcagcaagaagcccgcccggaaagagaagcccggctcagcaccctcagggggcccgtccaggctcagcagcagcagctcctccgagtctgggagcagcagctccagcgggtccagctctgacagcagtgactcagaatgaactggcttcggacagaacaggacagatggatgtcgcacacgccgagactctgccgtacccctctgtggttcatattactacttctgttccatggtgtgcaggtctgcttcttaattcagtgttatgatatcttccagtttttgctttcataggtcagagatctatcttgtgtgtgcgttagacttgatgagaaggtgtgaactctgcagaaagtctcttcttcatcactgaattcagtcacttggagatgacaacttcaaatgctaacccgatgaccccagaaaaccgtgtgagattcgtaccgaagaaccttgtggaatccctttgcttaggcccaacctggtcgatagctcgagaaagaattttttccaaggaaatgtctcggatatgggtactgtatttgaaagctgttagctttgtcaacacgcattgtccttgtcatttgggccccgagctctgaccctcgtgtctgacgcggccacctctttctggaggggctgaggacagatgtgcctgcttgtggagaccaggctgggcctaagcgaagggtcatcgcagccccagcccggagcgtggagcccttggggggtggtcgggtgggatgtgcgttctccgctcgtggtgatgtcaggagctcctcggagggaacagagcggctgtgtatgcagcctgcaggtttccatacactgaagcttttacctcaactttaaaaaaaaaaaaagaagaaaagatttaaaaaaaaaaaaaaaggagactttttttgtaaggttcggcaattttctacggaagtccagcccgctgtgagtccccgcctggctagcgctgcccctgctgctgctgccgcgccacccttgggaaccacttcccgagcttatgtgtatataaatagttatttattaacatcattggtcatttttaaaaaaaagaaaataagaaaaaaccgcagaagaaatgcattcacacagtcgcagagatgcaggccttgccagtggtgtgccgggcgcgggtcctgtctggcggcggcctgtcgtctccaggtgctaactcctgccaccgcgcggtgctcacccacgtctgttcgcgcgctcgcccctgggtttgttgggttttttggtttttttctttgtggtttttttttttttttttttttgtatgaaacttggaggcttacaggtatagacagctttcagctacagcacattctaattttttattttgtttagttcttttgtattcacttctggtctctttaagactgttttaaaagaaatcaatttagggaaccccagttatataatataaactttgtaatctgagagaaaaaatgtatagtaaatctaagtcttgatttttaactttctattgtaaaaaataataatatacagagtttaatagaaggtgatgttttggttttgttttcccagaggctgccatatggtctttgagtacggggatgtcccaaactggcccaccaatgagcatggcggctccggccaggaatgccagagttagcctcccaggcttgcgggtggacatgcctgctccctgccagcctccagtggcctggccaggccctcccgagcctgtctgccctccccaggggtggaggagtctctgggccccaggaggattccctcccggagactcgcacggtgctccctgctcacgcgttgtcacagttagtccggaaatgactgaaaccaggcattctcccggacctcagcgtgggggagcctccaggcagacgctgggtatggagctgtggtgtggtctgtcctgtatggtggccagtgctttctgccagcatttctggatggatatagggactatcattagtatcctaatacacggtgattttaaaacaaccataaaattgattcagagtccactgacccttacagatgtaggtatacccttactggagagggaactctgatgaggagatgctggtaaattatcattttttaaattgctggtgagtctgacacttggtgagttttcagccagtttgttaaacttttaattaagttttgtttataataaaaatataaatggatttgaaagtttccattttttaaagttaccctcgttttcaaaggtattttctaaacagatctttaatggactatttaaaccgaatttaaggaattcacacacgacagttgacaggtcttcacgcaggctggttggtaacgtgctgccagcacagggctgggtgatacgtacaccctaagccgggggtgcctggggctggggggcgctccttgcaatgcccctccagccacagggcagtgaggtgctgcctgtgtgagccgtcgggggagcggccggctgtgggggcagcgcagcaggagcatcgtggggcctttccttctcggctggttctctgtgacggtggcgtcggctcgcctctgctcctttcatctagaaagaagccactgaccctgacagcccacggcgggtacactgagcagctgcattggtgctgtcacttttttaaggctttctgtccagacttcaacactggtttcttttcagagtttcgaaggattaatgacttcctcagcgcccttgctggcgggctgagggtgacagtcacgtccgtttcttctgtattagaaggctgcggtgattcaattagattgtcccactgctgagacctgtagggcagcttctaacatgcttttttcaaggggagaggagtagtgacaagtcgtgtgtcggaattggatttgagaacactctgaatgacccctggaggccgagggggcaggcttcgggcgtgaactgaactccagacccctctttgtgttgggcagtgtcatcttgcttacaaactgtaagacacatttttttgtgtgtttgtttttgttgttgttcttttgcagcactcacgcctctgacagtcttttgggaaagagtaacacccacatacagaatttgtcacatccagagtagcactgttccttaatactggcataatgcttccaggaagtttttcttttttatatttaaaatgttacttttctgtatgatgtgcatgcaagtttaccgtaacttttcttaaactttttagtgccgtttctagtatattcctgtaaatgtcagttactgaaaatgagtccaatgtaagtagtttagcttgtttattgcaatgctggcctcaacacaacagaataaaaatggtagaaagtactctttgatgtttctggtaatcatggacccttctcctggggcatttgttttgttttcataataaaaagcaaaaaaaaaaaaaaaaaaaaaaaaaaaaaaaaaaaaaaaa

SEQ ID NO: 45 - protein, 726 aa, Homo sapiens
  MISC_FEATURE (1)..(726): Amino acid sequence Accession Number NP_031397
MSTATTVAPAGIPATPGPVNPPPPEVSNPSKPGRKTNQLQYMQNVVVKTLWKHQFAWPFYQPVDAIKLNLPDYHKIIKNPMDMGTIKKRLENNYYWSASECMQDFNTMFTNCYIYNKPTDDIVLMAQALEKIFLQKVAQMPQEEVELLPPAPKGKGRKPAAGAQSAGTQQVAAVSSVSPATPFQSVPPTVSQTPVIAATPVPTITANVTSVPVPPAAAPPPPATPIVPVVPPTPPVVKKKGVKRKADTTTPTTSAITASRSESPPPLSDPKQAKVVARRESGGRPIKPPKKDLEDGEVPQHAGKKGKLSEHLRYCDSILREMLSKKHAAYAWPFYKPVDAEALELHDYHDIIKHPMDLSTVKRKMDGREYPDAQGFAADVRLMFSNCYKYNPPDHEVVAMARKLQDVFEMRFAKMPDEPVEAPALPAPAAPMVSKGAESSRSSEESSSDSGSSDSEEERATRLAELQEQLKAVHEQLAALSQAPVNKPKKKKEKKEKEKKKKDKEKEKEKHKVKAEEEKKAKVAPPAKQAQQKKAPAKKANSTTTAGRQLKKGGKQASASYDSEEEEEGLPMSYDEKRQLSLDINRLPGEKLGRVVHIIQSREPSLRDSNPDEIEIDFETLKPTTLRELERYVKSCLQKKQRKPFSASGKKQAAKSKEELAQEKKKELEKRLQDVSGQLSSSKKPARKEKPGSAPSGGPSRLSSSSSSESGSSSSSGSSSDSSDSE

SEQ ID NO: 46 - DNA, 2539 bp, Homo sapiens
  misc_feature (1)..(2539): mRNA Accession Number BC032124.2 Homo sapiens BRD3R
gtttacaaacacgggctcccggcaggtgcgcgccgccccgcccgtgcgcggccggggttcgagggtggctcccgcgggcctcggggtgcccggacgggggctgcggtgctggctgcgtgcccgcttcttccatgccgtcctggggcaccggaaaatccgccgccaggcgctgtccccgacacgggctgtcgcctggttgggcccggaaatgggacgtcgcgctttctcagggagcgtagaagcagccagggcctctccaagccgctgctgtgacagaaagtgagtgagctgccggaggatgtccaccgccacgacagtcgcccccgcggggatcccggcgaccccgggccctgtgaacccaccccccccggaggtctccaaccccagcaagcccggccgcaagaccaaccagctgcagtacatgcagaatgtggtggtgaagacgctctggaaacaccagttcgcctggcccttctaccagcccgtggacgcaatcaaattgaacctgccggattatcataaaataattaaaaacccaatggatatggggactattaagaagagactagaaaataattattattggagtgcaagcgaatgtatgcaggacttcaacaccatgtttacaaattgttacatttataacaagcccacagatgacatagtgctaatggcccaagctttagagaaaatttttctacaaaaagtggcccagatgccccaagaggaagttgaattattaccccctgctccaaagggcaaaggtcggaagccggctgcgggagcccagagcgcaggtacacagcaagtggcggccgtgtcctctgtctccccagcgaccccctttcagagcgtgccccccaccgtctcccagacgcccgtcatcgctgccacccctgtaccaaccatcactgcaaacgtcacgtcggtcccagtccccccagctgccgccccacctcctcctgccacacccatcgtccccgtggtccctcctacgccgcctgtcgtcaagaaaaagggcgtgaagcggaaagcagacacaaccactcccacgacgtcggccatcactgccagccggagtgagtcgcccccgccgttgtcagaccccaagcaggccaaagtggtggcccggcgggagagtggtggccgccccatcaagcctcccaagaaggacctggaggacggcgaggtgccccagcacgcaggcaagaagggcaagctgtcggagcacctgcgctactgcgacagcatcctcagggagatgctatccaagaagcacgcggcctacgcctggcccttctacaagccagtggatgccgaggccctggagctgcacgactaccacgacatcatcaagcacccgatggacctcagcaccgtgaaaaggaagatggatggccgagagtacccagacgcacagggctttgctgctgatgtccggctgatgttctcgaattgctacaaatacaatcccccagaccacgaggttgtggccatggcccggaagctccaggacgtgtttgagatgaggtttgccaagatgccagatgagcccgtggaggcaccggcgctgcctgcccccgcggcccccatggtgagcaagggcgctgagagcagccgtagcagtgaggagagctcttcggactcaggcagctcggactcggaggaggagcgggccaccaggctggcggagctgcaggagcagctgaaggccgtgcacgagcagctggccgccctgtctcaggccccagtaaacaaaccaaagaagaagaaggagaagaaggagaaggagaagaagaagaaggacaaggagaaggagaaggagaagcacaaagtgaaggccgaggaagagaagaaggccaaggtggctccgcctgccaagcaggctcagcagaagaaggctcctgccaagaaggccaacagcacgaccacggccggcagagatcatttcttgacctgtggagtttgagacgcctatggggtgtagagaggaacgaacctctgtaattgtttcctggccaagggctggaaaccccgcagctgggagcgacttttctaaccttggattttctgccttggggcaccactttgggaagaaagcttggtcccagagagcagcctgctgttgggaggaaggggtgtgtgcagtgggctcccacggcaggtagacggagactcaacaccacgttgctctgtctcctgccccagacagctgaagaaaggcggcaagcaggcatctgcctcctacgactcagaggaagaggaggagggcctgcccatgagctacgatgaaaagcgccagcttagcctggacatcaaccggctgcccggggagaagctgggccgggtagtgcacatcatccaatctcgggagccctcgctcagggactccaaccccgacgagatagaaattgactttgagactctgaaacccccccctttgcgggaactggagagatatgtcaagtcttgtttacagaaaaagcaaaggaaaccgttctgtaaaaaaaaaaaaaaaaaaaaaaaaaaaaaaaaaaaa

SEQ ID NO: 47 - protein, 556 aa, Homo sapiens
  MISC_FEATURE (1)..(556): Amino Acid Accession Number AAH32124.1 Homo sapiens BRD3R
MSTATTVAPAGIPATPGPVNPPPPEVSNPSKPGRKTNQLQYMQNVVVKTLWKHQFAWPFYQPVDAIKLNLPDYHKIIKNPMDMGTIKKRLENNYYWSASECMQDFNTMFTNCYIYNKPTDDIVLMAQALEKIFLQKVAQMPQEEVELLPPAPKGKGRKPAAGAQSAGTQQVAAVSSVSPATPFQSVPPTVSQTPVIAATPVPTITANVTSVPVPPAAAPPPPATPIVPVVPPTPPVVKKKGVKRKADTTTPTTSAITASRSESPPPLSDPKQAKVVARRESGGRPIKPPKKDLEDGEVPQHAGKKGKLSEHLRYCDSILREMLSKKHAAYAWPFYKPVDAEALELHDYHDIIKHPMDLSTVKRKMDGREYPDAQGFAADVRLMFSNCYKYNPPDHEVVAMARKLQDVFEMRFAKMPDEPVEAPALPAPAAPMVSKGAESSRSSEESSSDSGSSDSEEERATRLAELQEQLKAVHEQLAALSQAPVNKPKKKKEKKEKEKKKKDKEKEKEKHKVKAEEEKKAKVAPPAKQAQQKKAPAKKANSTTTAGRDHFLTCGV

SEQ ID NO: 48 - protein, 21 aa, Artificial sequence
  Histone H3 N-terminal peptide biotinylated
  MISC_FEATURE (21)..(21): terminal biotinylated lysine
ARTKQTARKSTGGKAPRKQLK

SEQ ID NO: 49 - protein, 21 aa, Artificial sequence
  Histone H3 K9ac peptide biotinylated
  misc_feature (9)..(9): Acetylated lysine
  misc_feature (21)..(21): Biotinylated lysine
  misc_feature (21)..(21): Biotinylated termnal lysine
ARTKQTARKSTGGKAPRKQLK

SEQ ID NO: 50 - protein, 21 aa, Artificial sequence
  Histone H3 K14ac peptide biotinylated
  MISC_FEATURE (14)..(14): Acetylated lysine
  MISC_FEATURE (21)..(21): Biotinylated terminal  lysine
ARTKQTARKSTGGKAPRKQLK

SEQ ID NO: 51 - protein, 21 aa, Artificial sequence
  Histone H3 K9, K14ac peptide biotinylated
  MISC_FEATURE (9)..(9): Acetylated lysine
  MISC_FEATURE (14)..(14): Acetylated lysine
  MISC_FEATURE (21)..(21): Biotinylated termnal lysine
ARTKQTARKSTGGKAPRKQLK

SEQ ID NO: 52 - protein, 23 aa, Artificial sequence
  Histone H4 N-terminal peptide biotinylated
  MISC_FEATURE (1)..(1): Acetylated N-terminal serine
  MISC_FEATURE (23)..(23): PEG-biotnylated terminal arginine
SGRGKGGKGLGKGGAKRHRKVLR

SEQ ID NO: 53 - protein, 23 aa, Artificial sequence
  Histone H4 K5ac peptide biotinylated
  MISC_FEATURE (1)..(1): Acetylated N-terminal serine
  MISC_FEATURE (5)..(5): Acetylated lysine
  MISC_FEATURE (23)..(23): Peg-Biotinylated terminal  arginine
SGRGKGGKGLGKGGAKRHRKVLR

SEQ ID NO: 54 - protein, 23 aa, Artificial sequence
  Histone H4 K8ac peptide biotinylated
  misc_feature (1)..(1): Acetylated N-termnal serine
  misc_feature (8)..(8): Acetylated lysine
  misc_feature (23)..(23): Peg-biotinylated terminal arginine
SGRGKGGKGLGKGGAKRHRKVLR

SEQ ID NO: 55 - protein, 23 aa, Artificial sequence
  Histone H4 K12ac peptide biotinylated
  MISC_FEATURE (1)..(1): Acetylated N-termnal serine
  MISC_FEATURE (12)..(12): Acetylated lysine
  MISC_FEATURE (23)..(23): Peg-biotinylated terminal arginine
SGRGKGGKGLGKGGAKRHRKVLR

SEQ ID NO: 56 - protein, 23 aa, Artificial sequence
  Histone H4 K16ac peptide biotinylated
  MISC_FEATURE (1)..(1): Acteylated N-terminal serine
  MISC_FEATURE (16)..(16): Acteylated lysine
  MISC_FEATURE (23)..(23): Peg-biotinylated terminal arginine
SGRGKGGKGLGKGGAKRHRKVLR

SEQ ID NO: 57 - protein, 23 aa, Artificial sequence
  Histone H4 K5, K8, K12, K16ac peptide biotinylated
  MISC_FEATURE (1)..(1): Acteylated N-terminal serine
  MISC_FEATURE (5)..(5): Acteylated lysine
  MISC_FEATURE (8)..(8): Acteylated lysine
  MISC_FEATURE (12)..(12): Acteylated lysine
  MISC_FEATURE (16)..(16): Acteylated lysine
  MISC_FEATURE (23)..(23): Peg-biotinylated terminal arginine
SGRGKGGKGLGKGGAKRHRKVLR

SEQ ID NO: 58 - DNA, 9363 bp, Artificial sequnce
  Expression vector pLVH-EF1a.AcGFP-P2A-hOct4 (KH162) expression OCT4
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaactacaaaaacaaattacaaaaattcaaaattttcgggtttattacagggacagcagagatccagtttatcgatgtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacccggtcgccaccatggtgagcaagggcgccgagctgttcaccggcatcgtgcccatcctgatcgagctgaatggcgatgtgaatggccacaagttcagcgtgagcggcgagggcgagggcgatgccacctacggcaagctgaccctgaagttcatctgcaccaccggcaagctgcctgtgccctggcccaccctggtgaccaccctgagctacggcgtgcagtgcttctcacgctaccccgatcacatgaagcagcacgacttcttcaagagcgccatgcctgagggctacatccaggagcgcaccatcttcttcgaggatgacggcaactacaagtcgcgcgccgaggtgaagttcgagggcgataccctggtgaatcgcatcgagctgaccggcaccgatttcaaggaggatggcaacatcctgggcaataagatggagtacaactacaacgcccacaatgtgtacatcatgaccgacaaggccaagaatggcatcaaggtgaacttcaagatccgccacaacatcgaggatggcagcgtgcagctggccgaccactaccagcagaatacccccatcggcgatggccctgtgctgctgcccgataaccactacctgtccacccagagcgccctgtccaaggaccccaacgagaagcgcgatcacatgatctacttcggcttcgtgaccgccgccgccatcacccacggcatggatgagctgtacaagtccggactcagatctcgaggatctggagcaacaaacttctcactactcaaacaagcaggtgacgtggaggagaatcccggccccatggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggtgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctctgtcaccactctgggctctcccatgcattcaaactgataaggatccaccggatctaggcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggtttttccagtcacacctcaggtacctttaagaccaatgacttacaaggcagctgtagatcttagccactttttaaaagaaaagaggggactggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattgctagcgttttaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctagtggtttcatttactctaaacctgtgattcctctgaattattttcattttaaagaaattgtatttgttaaatatgtactacaaacttagtagtttttaaagaaattgtatttgttaaatatgtactacaaacttagtagt

SEQ ID NO: 59 - DNA, 9234 bp, Artificial sequence
  Expression vector pLVH-EF1a.AcGFP-P2A- hSox2(KH163) expressing Sox2
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaactacaaaaacaaattacaaaaattcaaaattttcgggtttattacagggacagcagagatccagtttatcgatgtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacccggtcgccaccatggtgagcaagggcgccgagctgttcaccggcatcgtgcccatcctgatcgagctgaatggcgatgtgaatggccacaagttcagcgtgagcggcgagggcgagggcgatgccacctacggcaagctgaccctgaagttcatctgcaccaccggcaagctgcctgtgccctggcccaccctggtgaccaccctgagctacggcgtgcagtgcttctcacgctaccccgatcacatgaagcagcacgacttcttcaagagcgccatgcctgagggctacatccaggagcgcaccatcttcttcgaggatgacggcaactacaagtcgcgcgccgaggtgaagttcgagggcgataccctggtgaatcgcatcgagctgaccggcaccgatttcaaggaggatggcaacatcctgggcaataagatggagtacaactacaacgcccacaatgtgtacatcatgaccgacaaggccaagaatggcatcaaggtgaacttcaagatccgccacaacatcgaggatggcagcgtgcagctggccgaccactaccagcagaatacccccatcggcgatggccctgtgctgctgcccgataaccactacctgtccacccagagcgccctgtccaaggaccccaacgagaagcgcgatcacatgatctacttcggcttcgtgaccgccgccgccatcacccacggcatggatgagctgtacaagtccggactcagatctcgaggatctggagcaacaaacttctcactactcaaacaagcaggtgacgtggaggagaatcccggccccatgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatgtgataaggatccaccggatctaggcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggtttttccagtcacacctcaggtacctttaagaccaatgacttacaaggcagctgtagatcttagccactttttaaaagaaaagaggggactggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattgctagcgttttaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctagtggtttcatttactctaaacctgtgattcctctgaattattttcattttaaagaaattgtatttgttaaatatgtactacaaacttagtagtttttaaagaaattgtatttgttaaatatgtactacaaacttagtagt

SEQ ID NO: 60 - DNA, 9690 bp, Artificial sequence
  Expression vector pLVH-EF1a.AcGFP-P2A- hKlf4(KH164) expression Klf4
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaactacaaaaacaaattacaaaaattcaaaattttcgggtttattacagggacagcagagatccagtttatcgatgtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacccggtcgccaccatggtgagcaagggcgccgagctgttcaccggcatcgtgcccatcctgatcgagctgaatggcgatgtgaatggccacaagttcagcgtgagcggcgagggcgagggcgatgccacctacggcaagctgaccctgaagttcatctgcaccaccggcaagctgcctgtgccctggcccaccctggtgaccaccctgagctacggcgtgcagtgcttctcacgctaccccgatcacatgaagcagcacgacttcttcaagagcgccatgcctgagggctacatccaggagcgcaccatcttcttcgaggatgacggcaactacaagtcgcgcgccgaggtgaagttcgagggcgataccctggtgaatcgcatcgagctgaccggcaccgatttcaaggaggatggcaacatcctgggcaataagatggagtacaactacaacgcccacaatgtgtacatcatgaccgacaaggccaagaatggcatcaaggtgaacttcaagatccgccacaacatcgaggatggcagcgtgcagctggccgaccactaccagcagaatacccccatcggcgatggccctgtgctgctgcccgataaccactacctgtccacccagagcgccctgtccaaggaccccaacgagaagcgcgatcacatgatctacttcggcttcgtgaccgccgccgccatcacccacggcatggatgagctgtacaagtccggactcagatctcgaggatctggagcaacaaacttctcactactcaaacaagcaggtgacgtggaggagaatcccggccccatggctgtcagcgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagaggcatttttaaggatccaccggatctaggcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggtttttccagtcacacctcaggtacctttaagaccaatgacttacaaggcagctgtagatcttagccactttttaaaagaaaagaggggactggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattgctagcgttttaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctagtggtttcatttactctaaacctgtgattcctctgaattattttcattttaaagaaattgtatttgttaaatatgtactacaaacttagtagtttttaaagaaattgtatttgttaaatatgtactacaaacttagtagt

SEQ ID NO: 61 - DNA, 9194 bp, Artificial sequence
  Expression vector pLVH-EF1a-attB1-BRD3\-attB2\ (KH226) expressing BRD3
tagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaactacaaaaacaaattacaaaaattcaaaattttcgggtttattacagggacagcagagatccagtttatcgatgtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacccggtcgaattcaacaagtttgtacaaaaaagcaggctatgtccaccgccacgacagtcgcccccgcggggatcccggcgaccccgggccctgtgaacccaccccccccggaggtctccaaccccagcaagcccggccgcaagaccaaccagctgcagtacatgcagaatgtggtggtgaagacgctctggaaacaccagttcgcctggcccttctaccagcccgtggacgcaatcaaattgaacctgccggattatcataaaataattaaaaacccaatggatatggggactattaagaagagactagaaaataattattattggagtgcaagcgaatgtatgcaggacttcaacaccatgtttacaaattgttacatttataacaagcccacagatgacatagtgctaatggcccaagctttagagaaaatttttctacaaaaagtggcccagatgccccaagaggaagttgaattattaccccctgctccaaagggcaaaggtcggaagccggctgcgggagcccagagcgcaggtacacagcaagtggcggccgtgtcctctgtctccccagcgaccccctttcagagcgtgccccccaccgtctcccagacgcccgtcatcgctgccacccctgtaccaaccatcactgcaaacgtcacgtcggtcccagtccccccagctgccgccccacctcctcctgccacacccatcgtccccgtggtccctcctacgccgcctgtcgtcaagaaaaagggcgtgaagcggaaagcagacacaaccactcccacgacgtcggccatcactgccagccggagtgagtcgcccccgccgttgtcagaccccaagcaggccaaagtggtggcccggcgggagagtggtggccgccccatcaagcctcccaagaaggacctggaggacggcgaggtgccccagcacgcaggcaagaagggcaagctgtcggagcacctacgctactgcgacagcatcctcagggagatgctatccaagaagcacgcggcctacgcctggcccttctacaagccagtggatgccgaggccctggagctgcacgactaccacgacatcatcaagcacccgatggacctcagcaccgtgaaaaggaagatggatggccgagagtacccagacgcacagggctttgctgctgatgtccggctgatgttctcgaattgctacaaatacaatcccccagaccacgaggttgtggccatggcccggaagctccaggacgtgtttgagatgaggtttgccaagatgccagatgagcccgtggaggcaccggcgctgcctgcccctgcggcccccatggtgagcaagggcgctgagagcagccgtagcagtgaggagagctcttcggactcaggcagctcggactcggaggaggagcgggccaccaggctggcggagctgcaggagcagctgaaggccgtgcacgagcagctggccgccctgtctcaggccccagtaaacaaaccaaagaagaagaaggagaagaaggagaaggagaagaagaagaaggacaaggagaaggagaaggagaagcacaaagtgaaggccgaggaagagaagaaggccaaggtggctccgcctgccaagcaggctcagcagaagaaggctcctgccaagaaggccaacagcacgaccacggccggcagagatcatttcttgacctgtggagttacccagctttcttgtacaaagtggtgatccaccggatctaggcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggtttttccagtcacacctcaggtacctttaagaccaatgacttacaaggcagctgtagatcttagccactttttaaaagaaaagaggggactggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattgctagcgttttaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctagtggtttcatttactctaaacctgtgattcctctgaattattttcattttaaagaaattgtatttgttaaatatgtactacaaacttagtagtttttaaagaaattgtatttgttaaatatgtactacaaacttagtagttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacag

(19) United States
(12) Patent Application Publication
(10) Pub. No.: US 2018/0163180 A1
(43) Pub. Date: Jun. 14, 2018

(54) METHOD OF ENHANCING SOMATIC CELL REPROGRAMMING WITH THE ACETYLLYSINE READER BRD3R

(71) Applicant: THE UAB RESEARCH FOUNDATION, INC ., BIRMINGHAM, AL (US)

(72) Inventors: KEJIN HU, VESTAVIA HILLS, AL (US); ZHICHENG SHAO, BELMONT, MA (US)

(73) Assignee: The UAB Research Foundation Inc., Birmingham, AL (US)

(21) Appl. No.: 15/571,078

(22) PCT Filed: Apr. 29, 2016
(86) PCT No.: PCT/US2016/030014

§ 371 Date: Nov. 1, 2017

Related U.S. Application Data
(60) Provisional application No. 62/155,622, filed on May 1, 2015.

(51) Int. Cl. C12N 5/074; C12N 15/86; C07K 14/47

(52) U.S. Cl. CPC C12N 5/0696; C12N 15/86; C07K 14/4702; C12N 2501/40; C12N 2506/1307; C12N 2501/602; C12N 2501/604; C12N 2740/15043; C12N 2510/00; C12N 2501/603

(57) ABSTRACT

A method of generating an induced pluripotent stem cell (iPSC) comprises introducing to an animal somatic cell at least one nuclear reprogramming inducing factor and a BRD3R polypeptide or at least one nucleic acid expressing the at least one nuclear reprogramming factor and the BRD3R-related polypeptide in the recipient somatic cell, and culturing said cell to generate an induced pluripotent stem cell (iPSC). The introduction of the BRD3R-related polypeptide into the recipient somatic cell can increase the efficiency of inducing the generation of an iPSC by the at least one nuclear reprogramming inducing factor.

### CROSS-REFERENCE TO RELATED APPLICATIONS

This application claims priority to U.S. Provisional Patent Application Ser. No. 62/155,622 entitled “METHOD OF ENHANCING SOMATIC CELL REPROGRAMMING WITH THE ACETYLLYSINE READER BRD3R” filed on May 1, 2015, the entirety of which is hereby incorporated by reference.

### FIELD OF THE DISCLOSURE

The present disclosure relates generally to methods of reprogramming a mammalian cell to generate a pluripotent stem cell. This disclosure further relates to the acetyllysine reader BRD3R gene expression product to enhance reprogramming of a mammalian cell.

### SEQUENCE LISTING

The present disclosure includes a sequence listing filed in electronic form as an ASCII.txt file entitled 2221042770_ST25, created on Apr. 28, 2016, the content of which is incorporated herein by reference in its entirety.

### BACKGROUND

Induced Pluripotent Stem Cell (iPSC) technology (or factor reprogramming), the generation of pluripotent stem cells (PSCs) by overexpression of transgenes, offers great opportunities for regenerative medicine and stem cell biology (Takahashi & Yamanaka (2006) Cell 126: 663-676). Understanding of the reprogramming process remains limited despite extensive improvement of iPSC technology and great efforts in deciphering this process (Hu K. (2014) Stem Cells Dev. 23: 1285-1300; Hu K. (2014) Stem Cells Dev. 23: 1301-1315; Hu & Slukvin (2013) Methods Mol. Biol. 997: 163-176; Hu et al., (2011) Blood 117: e109-119; Guo et al., (2014) Cell 156: 649-662). Compared to reprogramming via somatic cell nuclear transfer (SCNT) and cell fusion, factor reprogramming has a very low efficiency and slow kinetics, suggesting the existence of additional yet-to-be discovered reprogramming factors.

PSCs have a unique cell cycle structure characterized by a truncated G1 phase, lack of a G1 checkpoint, lack of CDK periodicity, and a greater portion of cells in S/G2/M phases as compared to somatic cells (White & Dalton (2005) Stem Cell Rev. 1: 131-138). During the reprogramming process, the pluripotent cell cycle structure has to be reset along with many other pluripotent features including differentiation potential, self-renewal, epigenetic landscape, transcriptome, and the unique morphologies of the pluripotent cells and their colonies.

Since the advent of SCNT, one consistent observation has been that only oocytes in the mitosis stage (metaphase II) possess sufficient reprogramming activity to clone animals successfully (Wakayama et al., (2000) Nat. Genet. 24: 108-109). Upon fertilization, such a reprogramming capacity becomes lost in the zygote, but it can be restored when a zygote is arrested in mitosis (Egli et al., (2007) Nature 447: 679-685). In addition, the donor nucleus in SCNT was recently demonstrated to have a mitotic advantage as well because donor chromatin arrested at mitosis are 100× easier to reprogram (Halley-Stott et al., (2014) PLoS Biol. 12: e1001914). The underlying molecular basis for both the potent reprogramming power and the higher reprogrammability of mitotic cells is unknown. Efforts have been made to investigate the role of acetyl epigenetics in reprogramming because of the importance of histone acetylation in PSCs and transcription controls, but these efforts have been restricted to the use of HDAC inhibitors (Liang et al., (2010) J. Biol. Chem. 285: 25516-25521). Here, the identification is now reported of an acetyllysine reader BRD3R, a BET bromodomain protein, as a novel robust reprogramming factor. Evidence is presented that BRD3R facilitates resetting of the pluripotent cell cycle structure and increases the number of reprogramming-privileged mitotic cells by upregulating as many as 128 mitotic genes through its continuous association with mitotic chromatin. At least 19 of these BRD3R-upregulated mitotic genes may constitute a novel expression fingerprint of PSCs.

### SUMMARY

The higher efficiency and faster kinetics observed in reprogramming with somatic cell nuclear transfer (SCNT) compared to factor reprogramming imply the existence of as yet unidentified reprogramming factors. In addition, both recipient cells and donor nuclei demonstrate a mitotic advantage in the traditional SCNT reprogramming, suggesting that mitotic factors play a critical role in reprogramming. An isoform of the bromodomain-containing 3 (BRD3), BRD3R (BRD3 with Reprogramming activity), has now been identified as a robust reprogramming factor that positively regulates mitosis during reprogramming via its continuous association with mitotic chromatin and its upregulation of a large number of mitotic genes without compromising the ARF-p53 surveillance pathway. Nineteen of the BRD3R-upregulated mitotic genes constitute a novel pluripotent molecular signature.

Accordingly, one aspect of the disclosure, therefore, encompasses embodiments of a method of generating an induced pluripotent stem cell (iPSC), said method comprising the steps of: introducing to an animal somatic cell at least one nuclear reprogramming inducing factor and a BRD3R polypeptide having an amino acid sequence having at least 90% sequence similarity to the amino acid sequence according to SEQ ID NO: 47, or at least one nucleic acid expressing said at least one nuclear reprogramming factor and said BRD3R-related polypeptide in the recipient somatic cell, and generating a population of induced pluripotent stem cells (iPSCs) by culturing the recipient somatic cell under conditions that promote the proliferation of said cell.

In some embodiments of this aspect of the disclosure the amino acid sequence can have at least 90% sequence similarity to the amino acid sequence according to SEQ ID NO: 47 and can be expressed from a recombinant expression vector comprising a nucleotide sequence encoding said amino acid sequence operably linked to a gene expression promoter

In some embodiments of this aspect of the disclosure the expression vector can be a lentivirus expression vector.

In some embodiments of this aspect of the disclosure the at least one nucleic acid expressing said at least one nuclear reprogramming factor can be inserted in a recombinant expression vector. In some embodiments the disclosure the expression vector is a lentivirus expression vector.

In embodiments of this aspect of the disclosure the introduction of said BRD3R-related polypeptide into the recipient somatic cell can increase the efficiency of inducing the generation of an iPSC by the at least one nuclear reprogramming inducing factor compared to when said BRD3R-related polypeptide is not introduced into the recipient somatic cell.

In some embodiments of this aspect of the disclosure the nuclear reprogramming inducing factor or a combination of said factors can be selected from the group consisting of: (1) OCT4, or a nucleic acid sequence that encodes the same; (2) SOX2, or a nucleic acid sequence that encodes the same; (3) KLF4, or a nucleic acid sequence that encodes the same; (4) OCT4 and SOX2, or nucleic acid sequences that encode the same; (5) OCT4 and KLF4, or nucleic acid sequences that encode the same; (6) SOX2 and KLF4, or nucleic acid sequences that encode the same; (7) OCT4, SOX2 and KLF4, or nucleic acid sequences that encode the same.

In some embodiments of this aspect of the disclosure the combination of nuclear reprogramming inducing factors of (4)-(7) can be expressed from a single nucleic acid sequence or individual nucleic acid sequences.

Another aspect of the disclosure encompasses embodiments of an expression vector comprising a nucleotide sequence encoding a polypeptide having an amino acid sequence having at least 90% sequence similarity to the amino acid sequence according to SEQ ID NO: 47, wherein said nucleotide sequence is operatively linked to a region of the expression vector that provides expression of the nucleotide sequence in a recipient cell.

In some embodiments of this aspect of the disclosure the expression vector further comprising at least one nucleic acid region encoding a nuclear reprogramming inducing factor or a combination of said factors, wherein said nucleotide sequence is operatively linked to a region of the expression vector that provides expression of the nucleotide sequence in a recipient cell.

In some embodiments of this aspect of the disclosure the nuclear reprogramming inducing factor or a combination of said factors can be selected from the group consisting of: (1) OCT4, or a nucleic acid sequence that encodes the same; (2) SOX2, or a nucleic acid sequence that encodes the same; (3) KLF4, or a nucleic acid sequence that encodes the same; (4) OCT4 and SOX2, or nucleic acid sequences that encode the same; (5) OCT4 and KLF4, or nucleic acid sequences that encode the same; (6) SOX2 and KLF4, or nucleic acid sequences that encode the same; (7) OCT4, SOX2 and KLF4.

In some embodiments of this aspect of the disclosure the expression vector is a lentivirus expression vector.

Another aspect of the disclosure encompasses embodiments of a modified animal somatic cell, wherein said cell can comprise a polypeptide having an amino acid sequence having at least 90% sequence similarity to the polypeptide BRD3R, or a heterologous nucleic acid expressing said BRD3R-related polypeptide.

In some embodiments of this aspect of the disclosure the modified animal somatic cell can be genetically modified by a heterologous nucleic acid expressing the BRD3R-related polypeptide.

In some embodiments of this aspect of the disclosure the modified animal somatic cell can be further modified by a heterologous nucleic acid expressing a nuclear reprogramming inducing factor or a combination of said factors selected from the group consisting of: (1) OCT4, or a nucleic acid sequence that encodes the same; (2) SOX2, or a nucleic acid sequence that encodes the same; (3) KLF4, or a nucleic acid sequence that encodes the same; (4) OCT4 and SOX2, or nucleic acid sequences that encode the same; (5) OCT4 and KLF4, or nucleic acid sequences that encode the same; (6) SOX2 and KLF4, or nucleic acid sequences that encode the same; (7) OCT4, SOX2 and KLF4, or nucleic acid sequences that encode the same.

In some embodiments of this aspect of the disclosure the combination of nuclear reprogramming inducing factors of (4)-(7) can be expressed from a single nucleic acid sequence or individual nucleic acid sequences.

### BRIEF DESCRIPTION OF THE DRAWINGS

Aspects of the present disclosure will be more readily appreciated upon review of the detailed description of its various embodiments, described below, when taken in conjunction with the accompanying drawings. The drawings are described in greater detail in the description and examples below.

FIGS. 1A-1E illustrate the identification of BRD3R as a robust human reprogramming factor.

FIG. 1A illustrates a schematic summary of the optimized screening protocol for the search of novel human reprogramming factor. D, days of reprogramming.

FIG. 1B illustrates the fold changes in numbers of ALP⁺ colonies for the 89 human kinase cDNAs in primary screen as compared to GFP control. #, genes selected for the secondary screen.

FIG. 10 illustrates the fold change for the 11 candidate genes in the secondary screen.

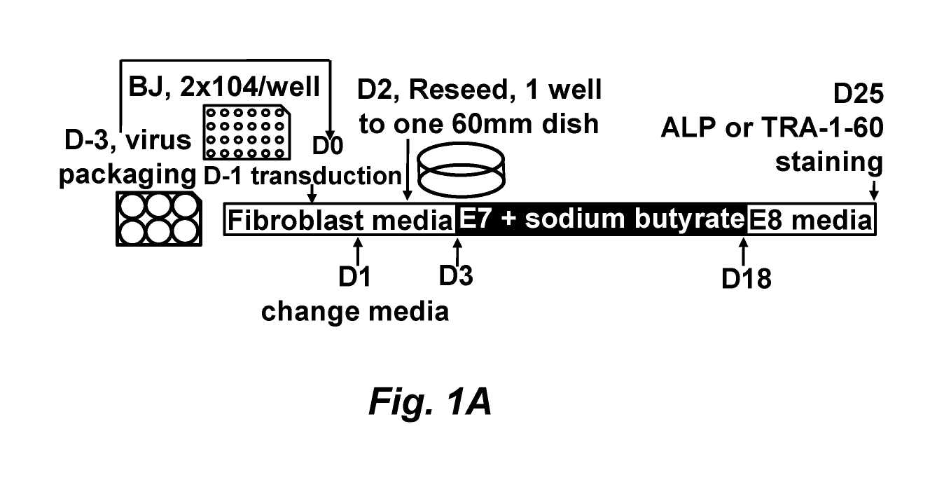

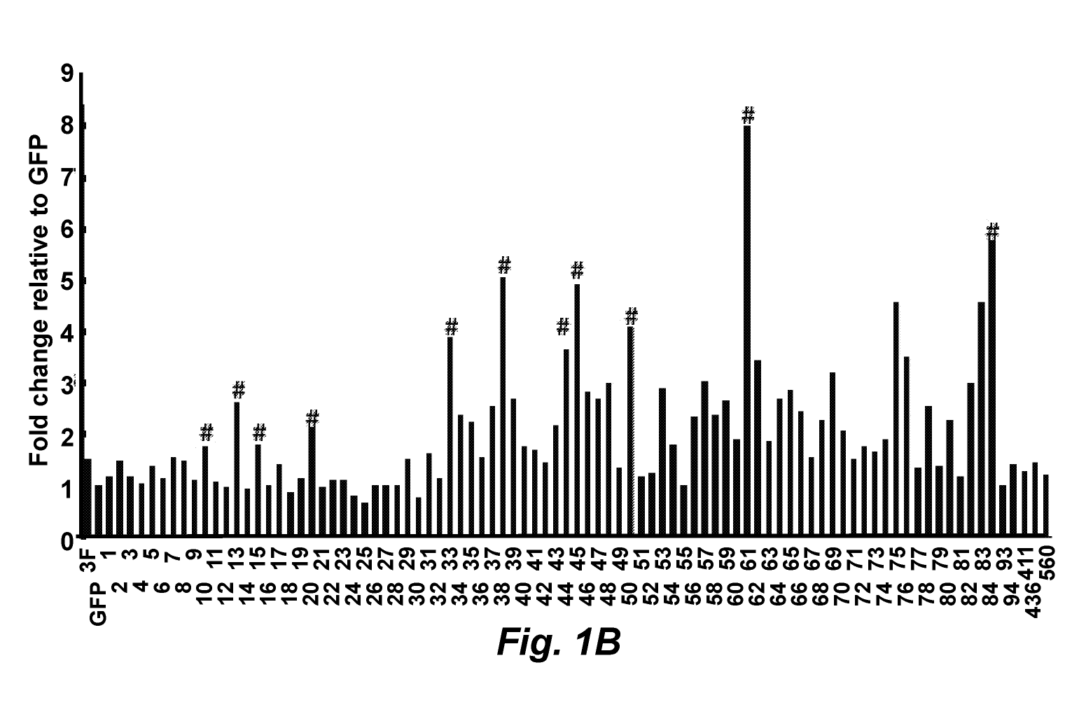

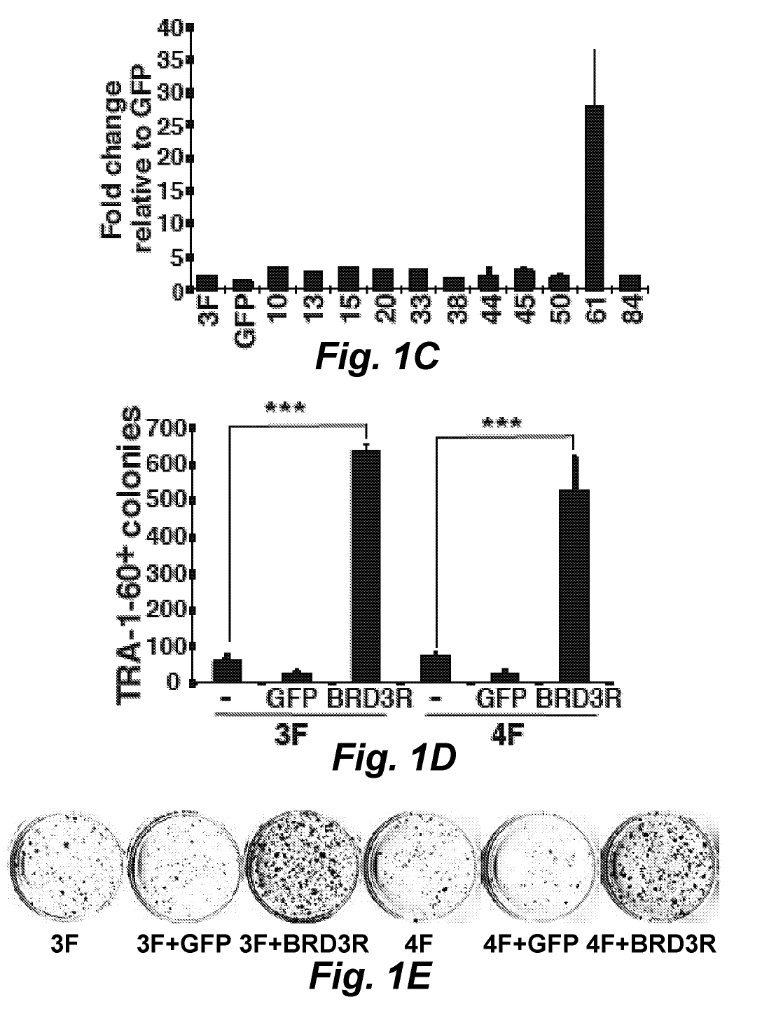

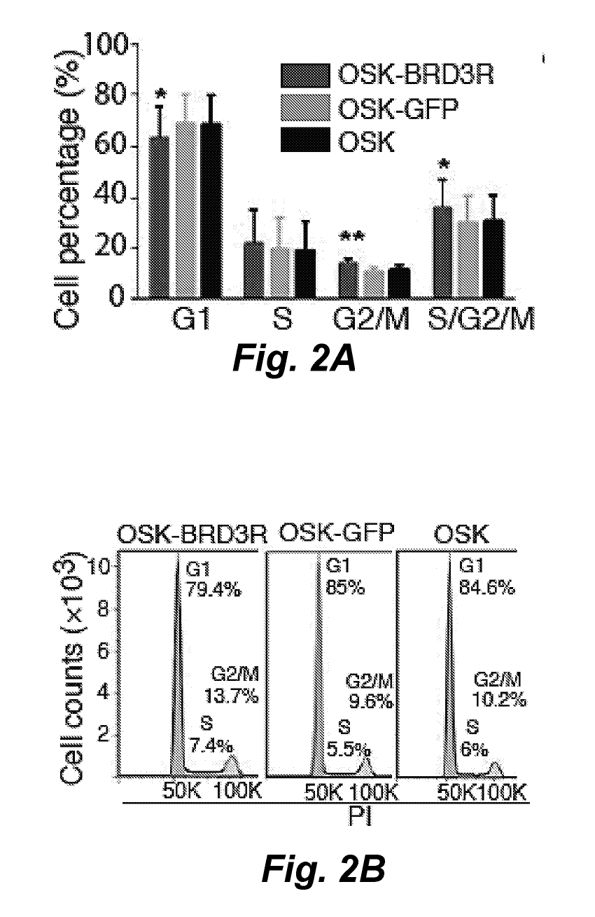

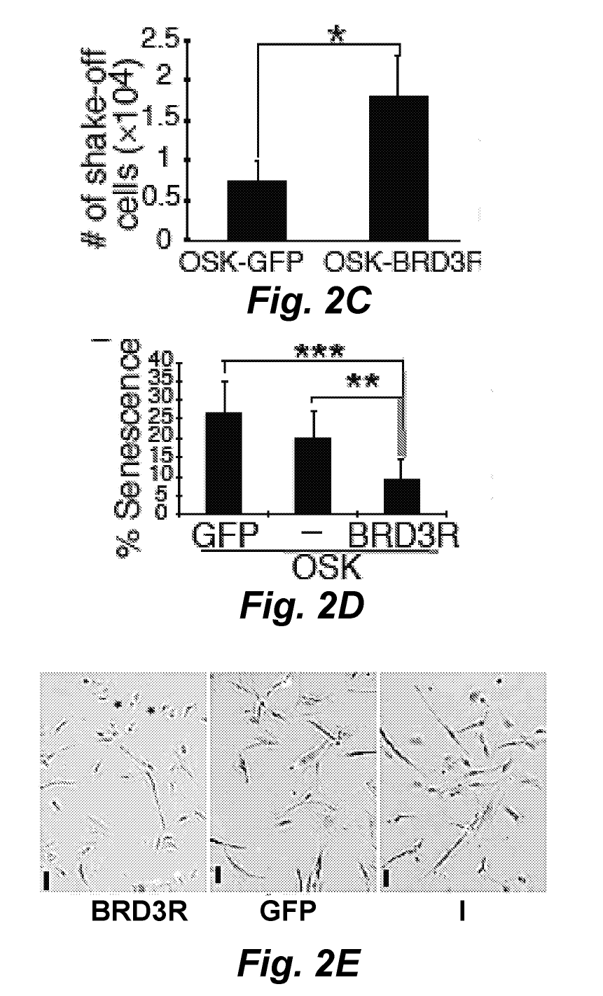

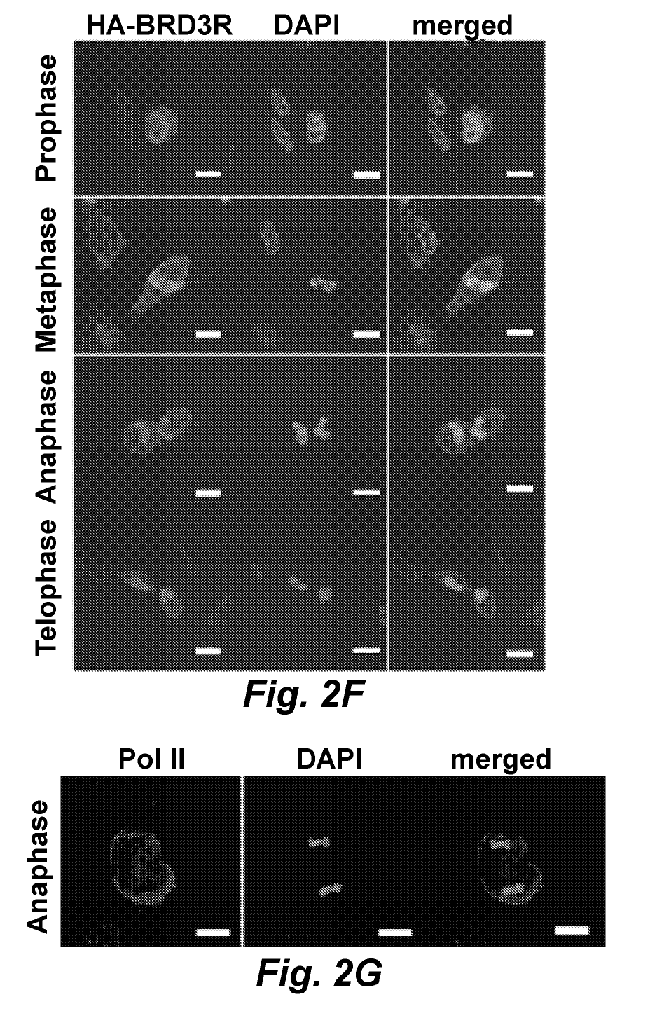

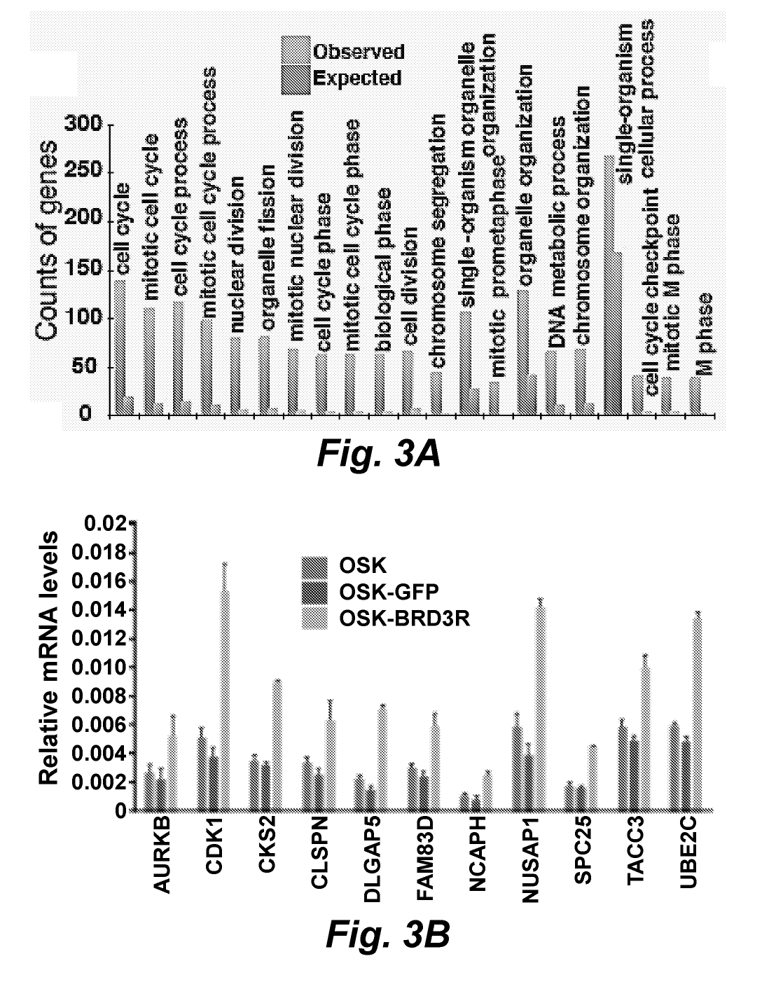

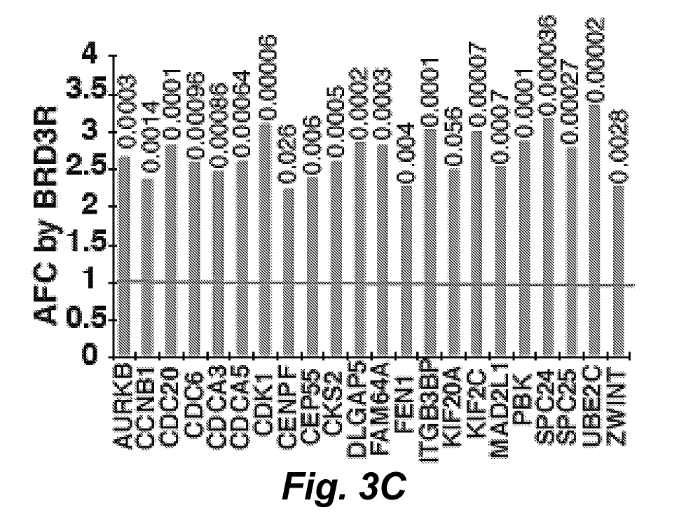

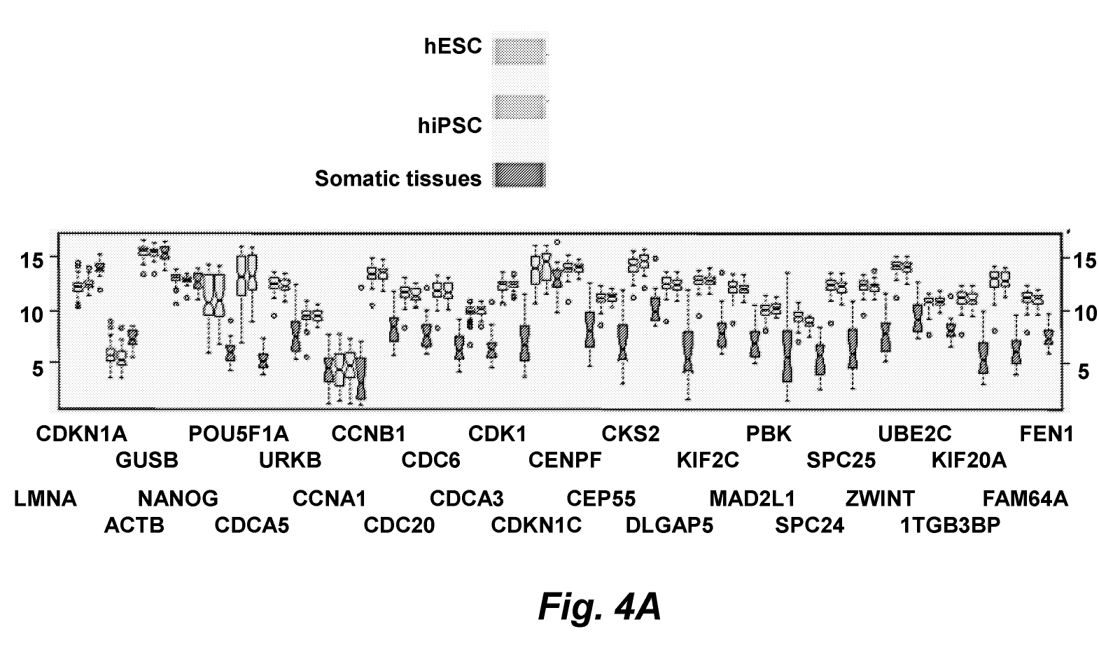

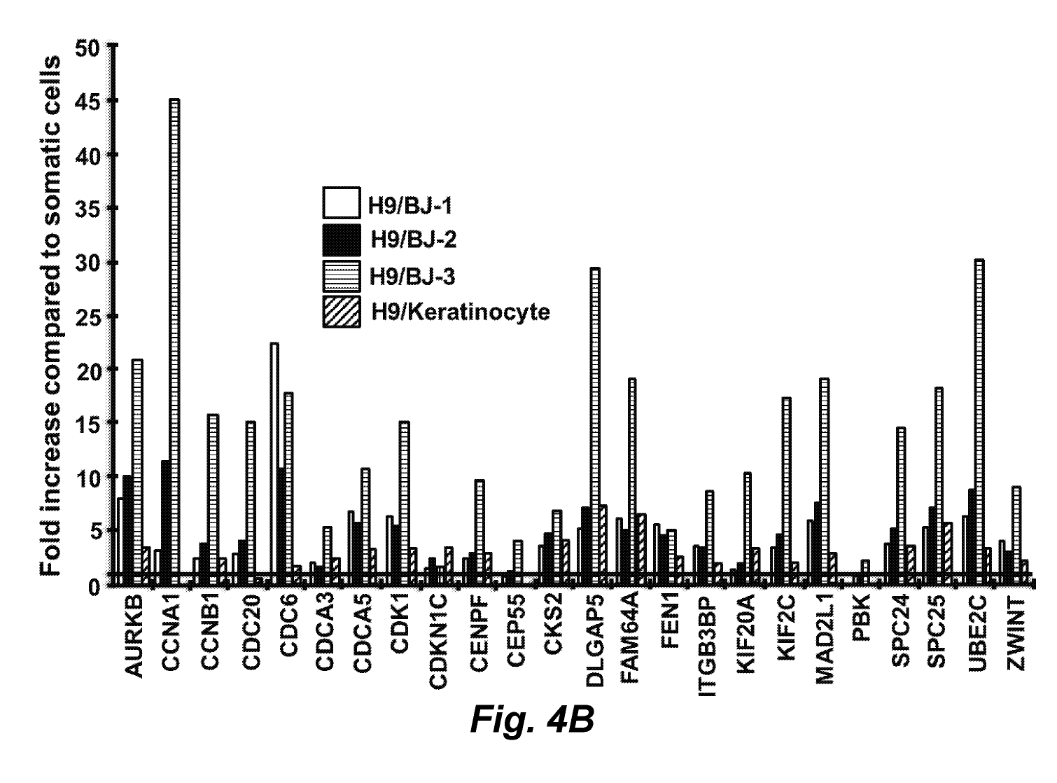

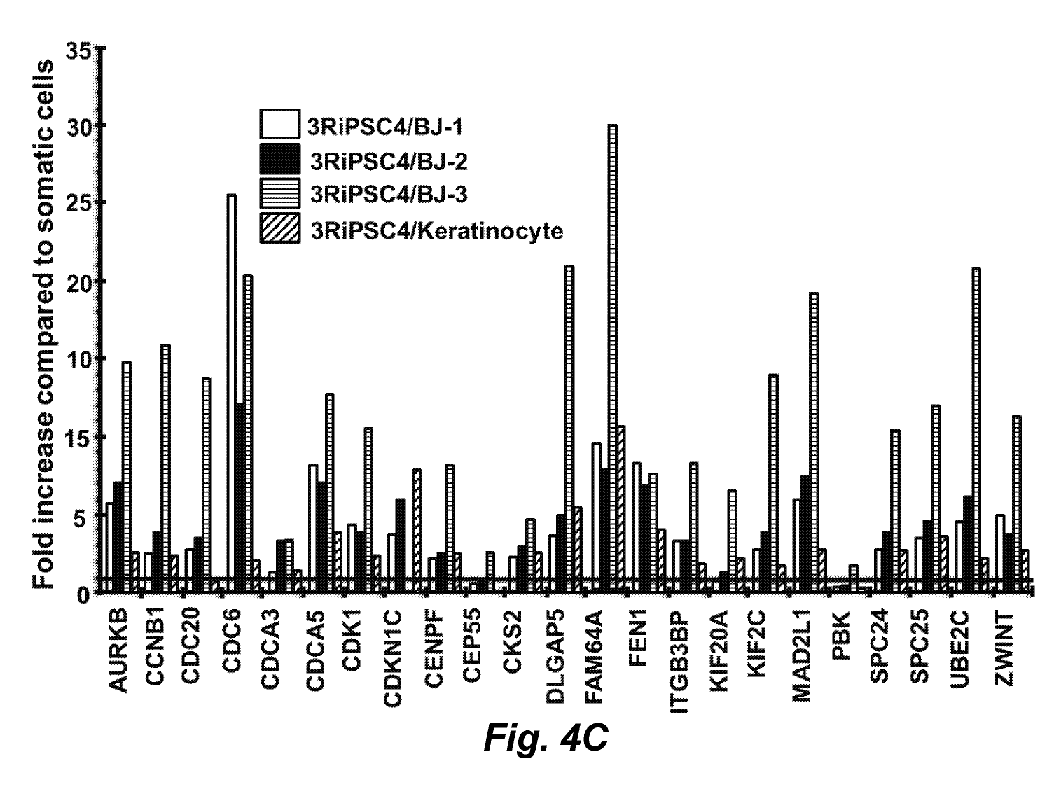

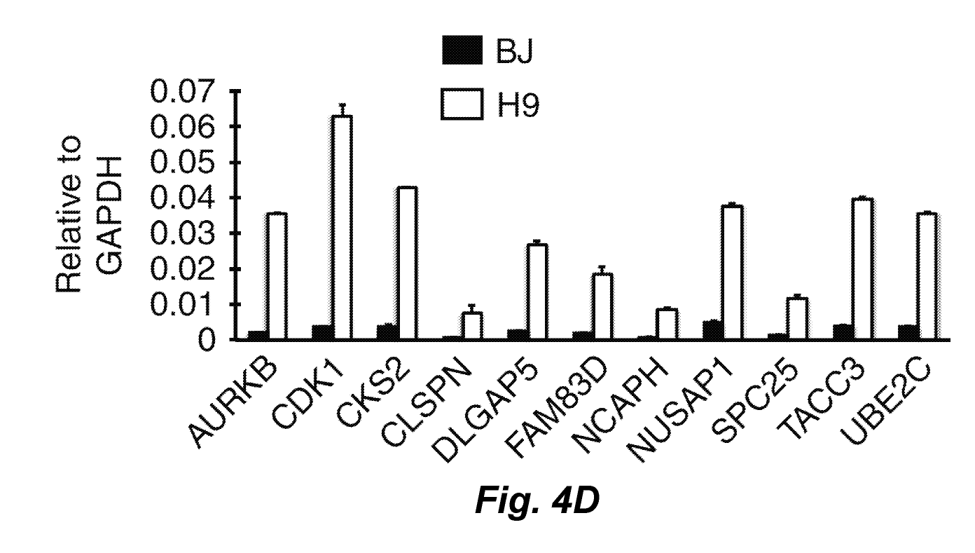

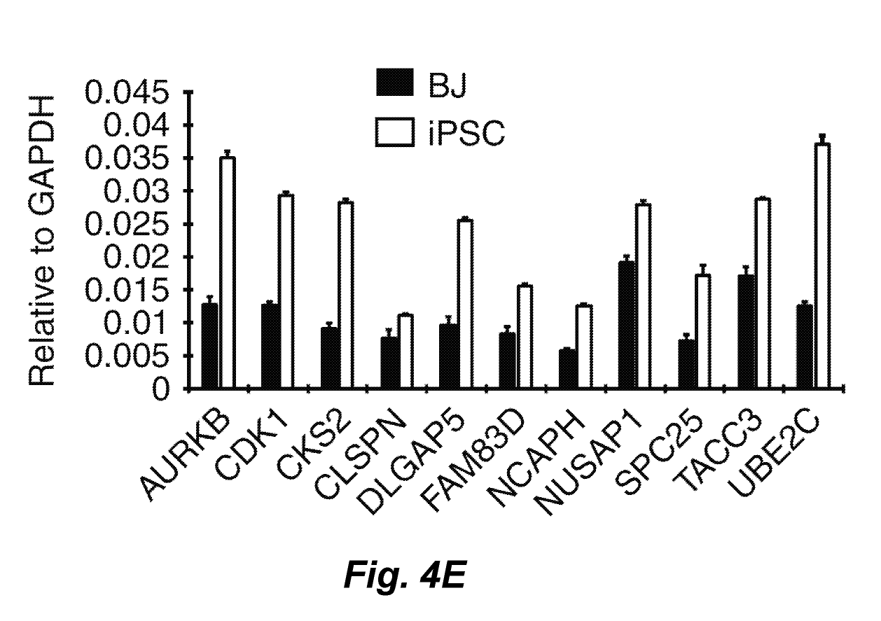

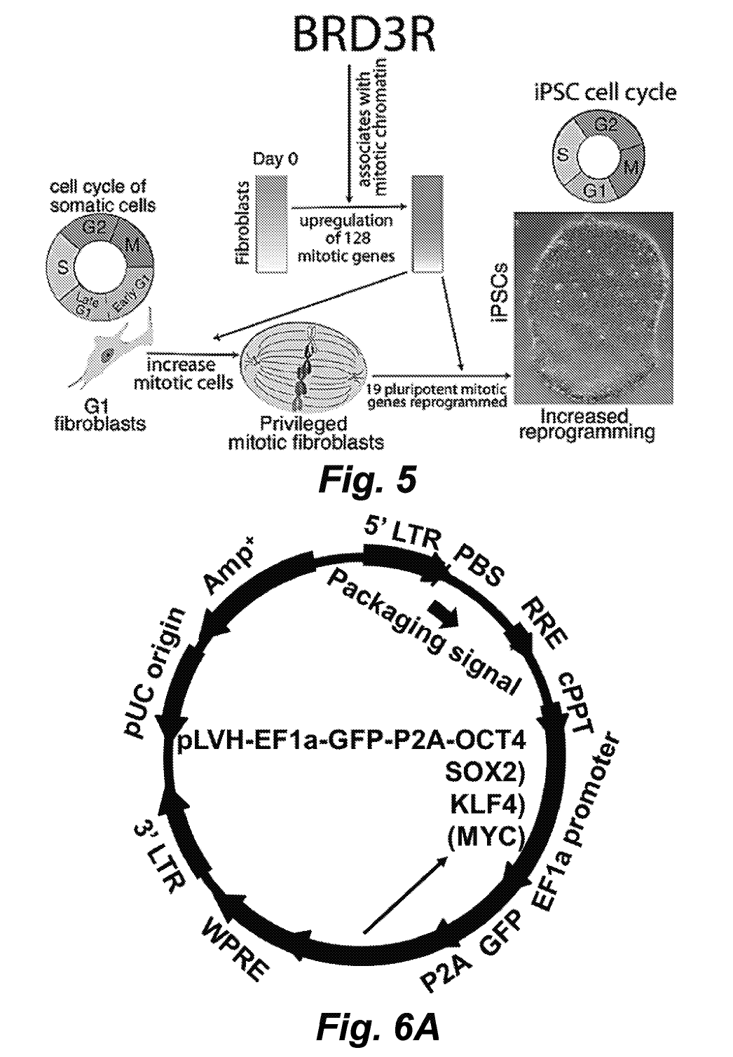

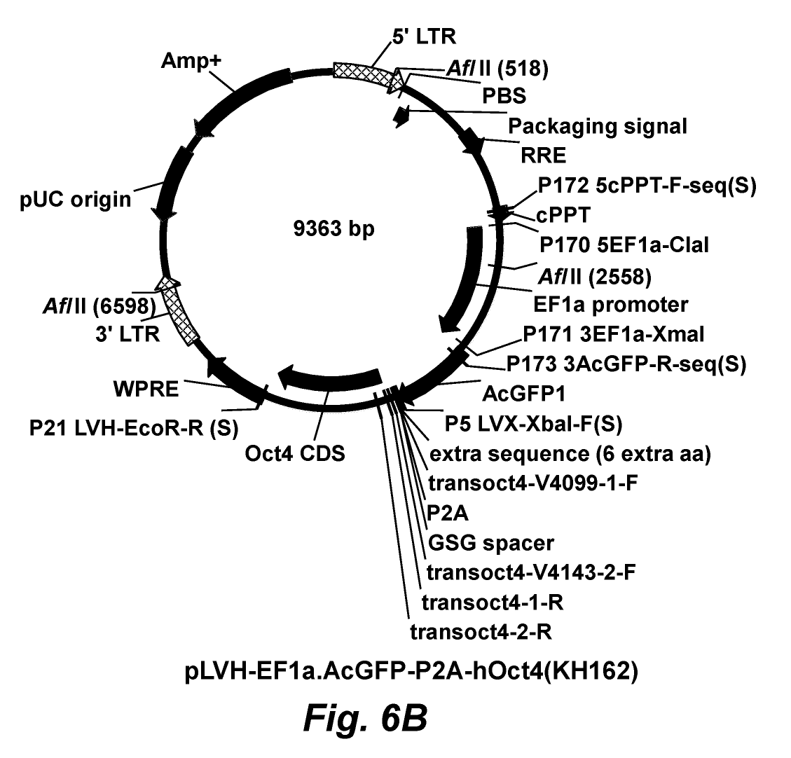

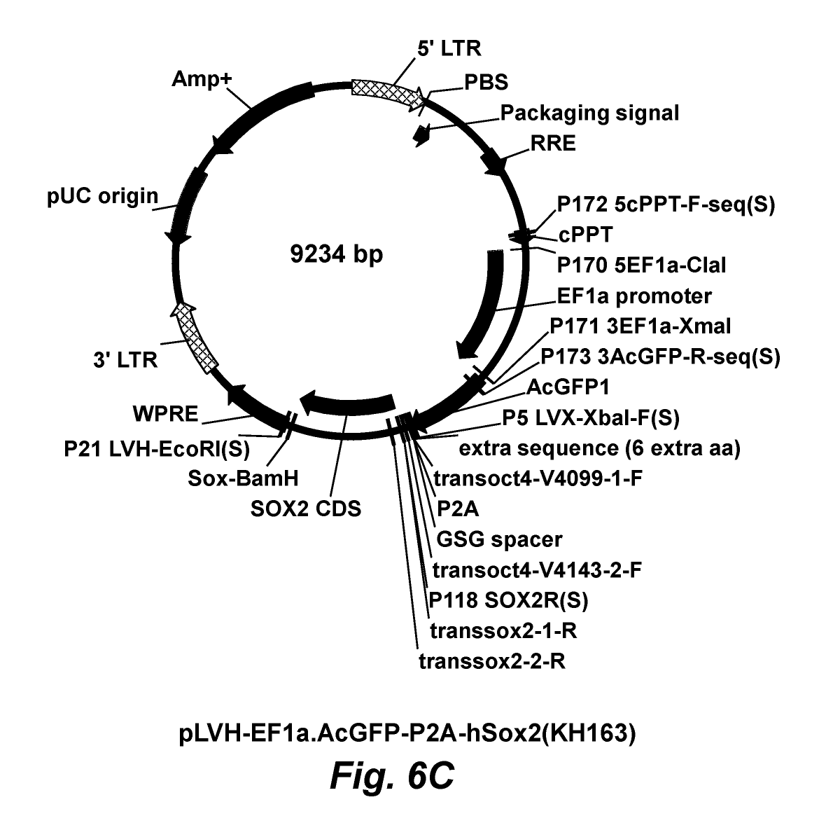

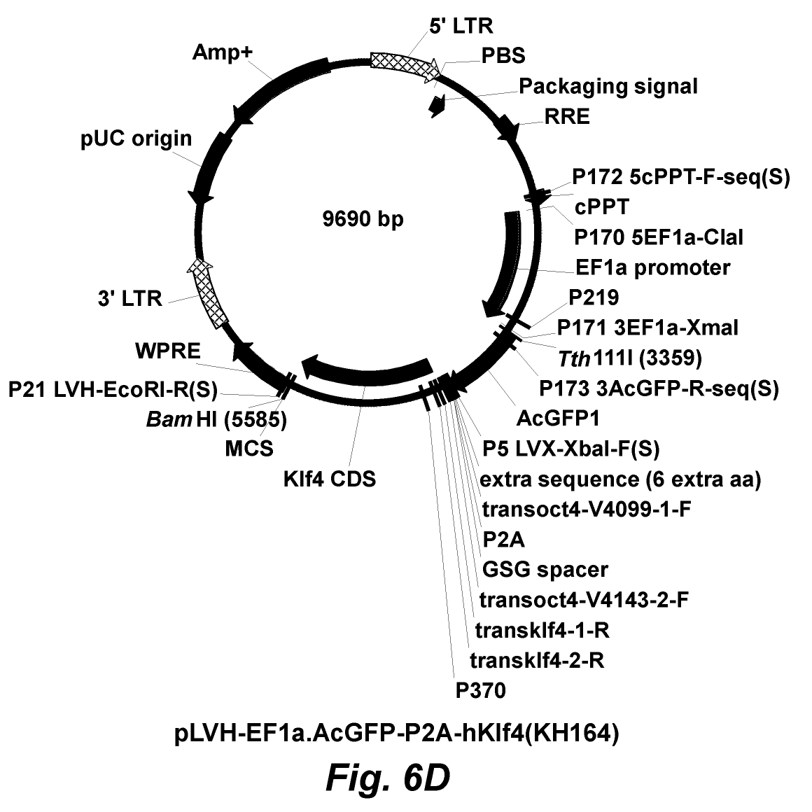

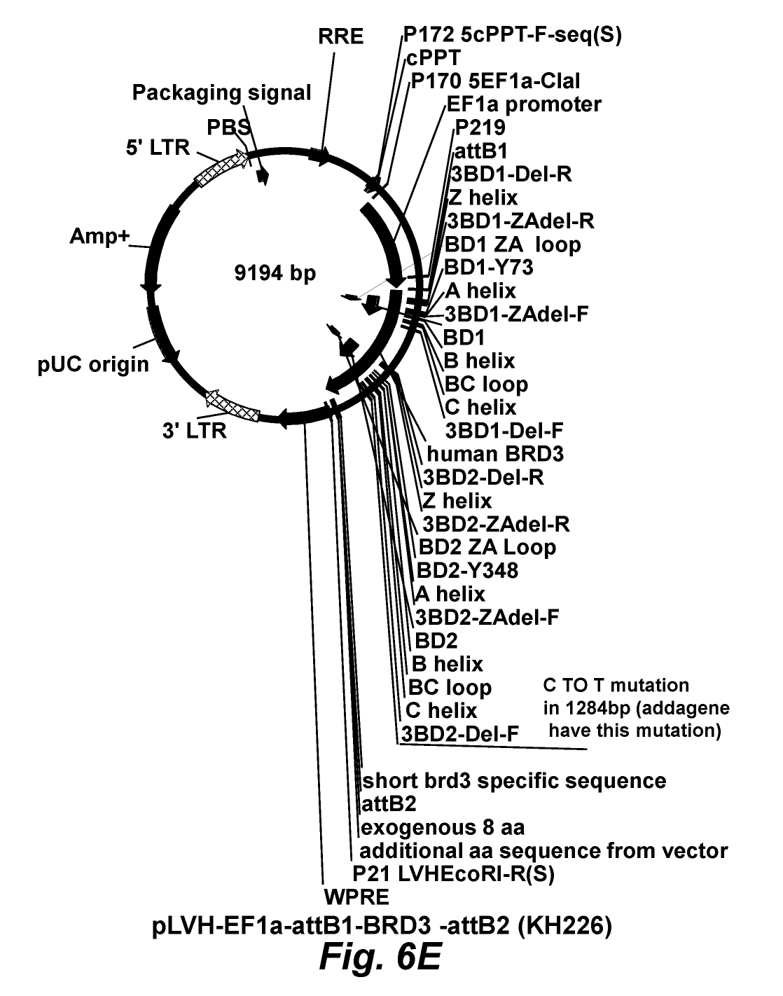

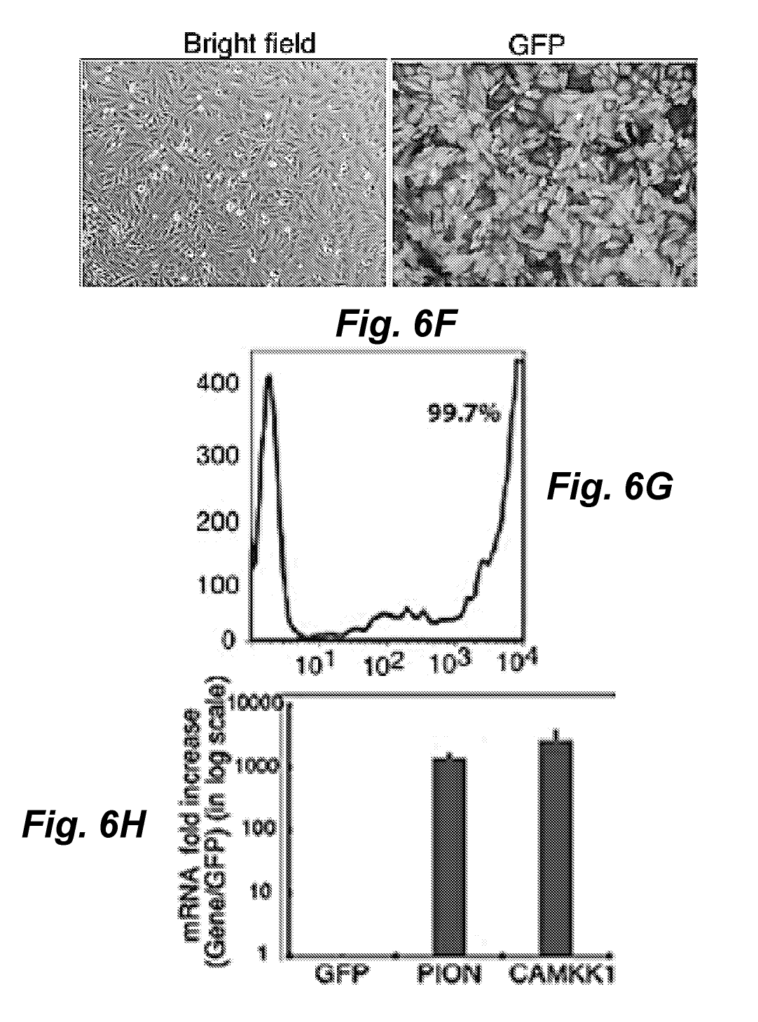

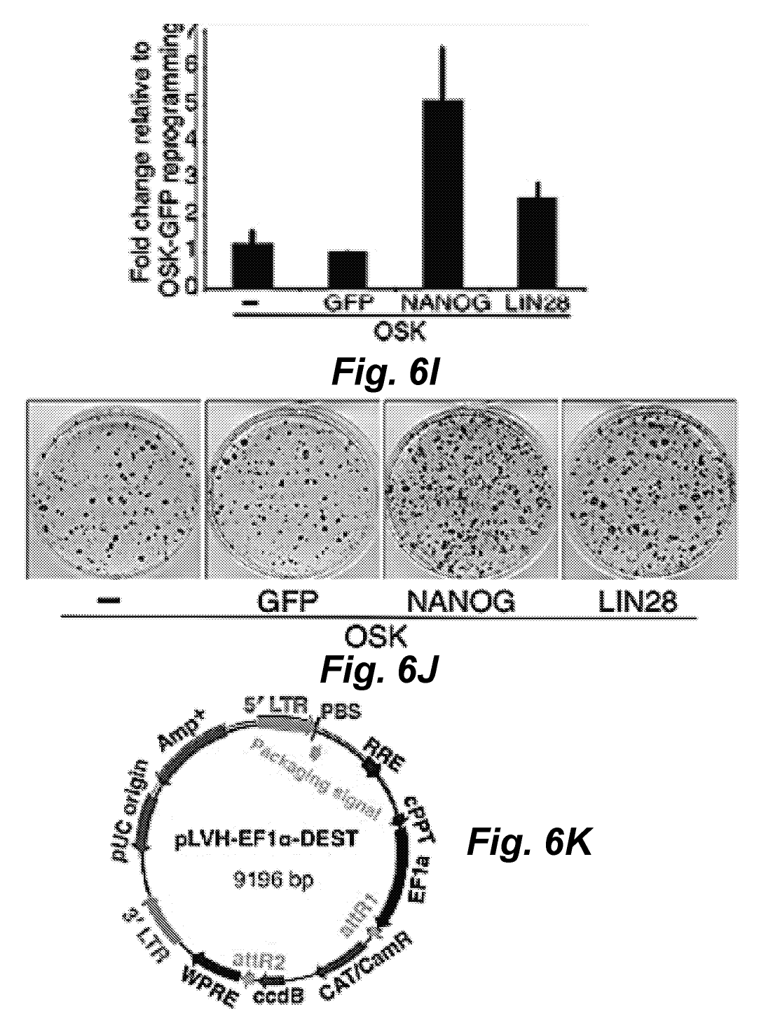

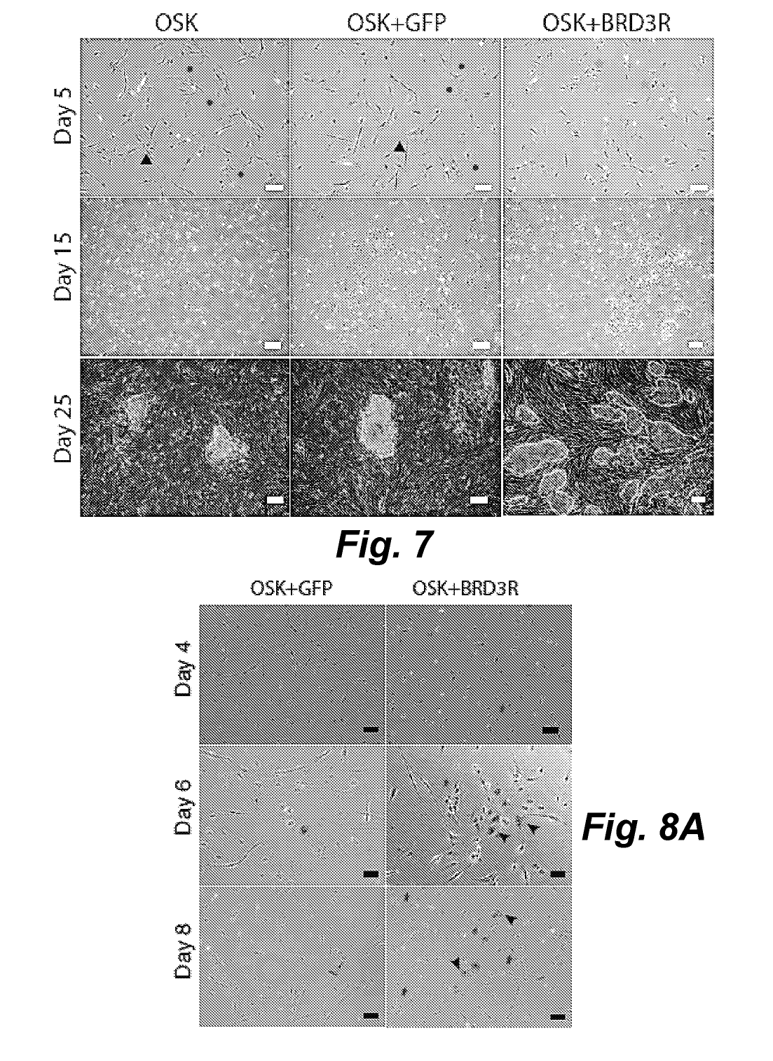

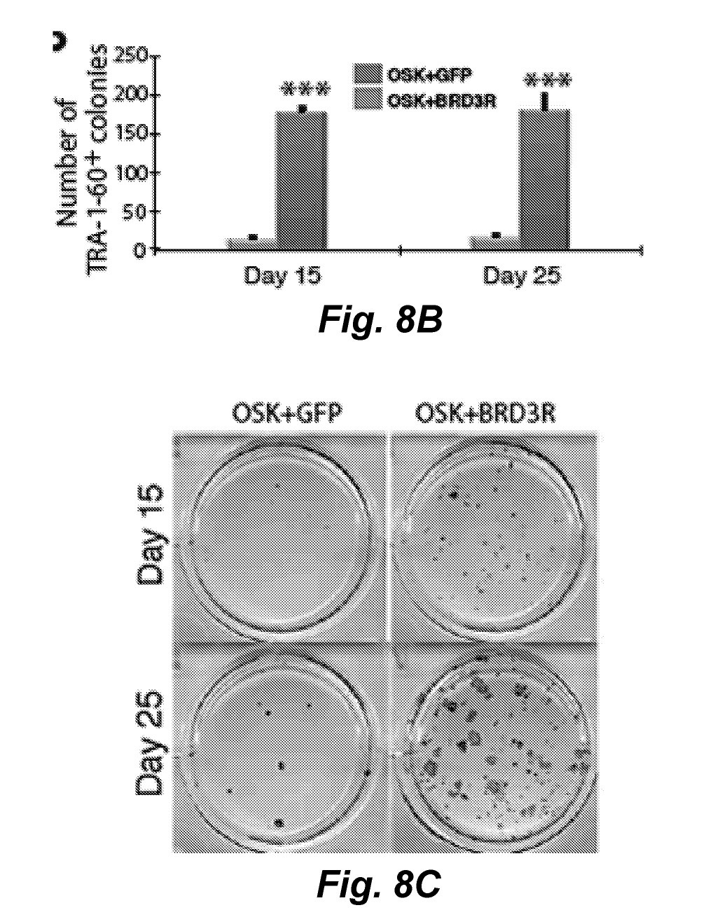

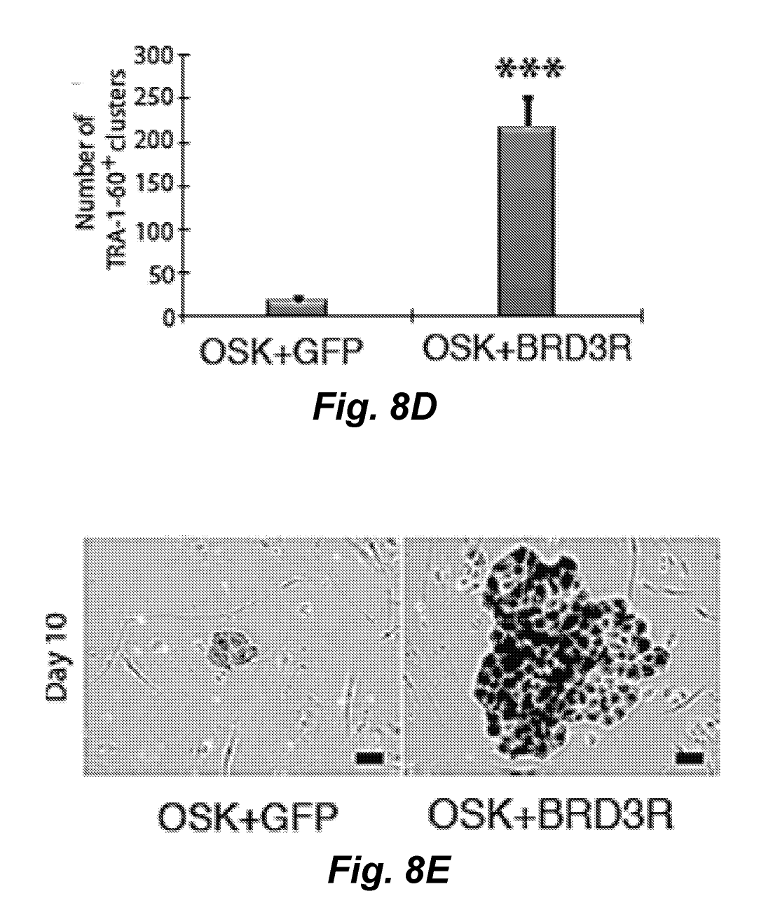

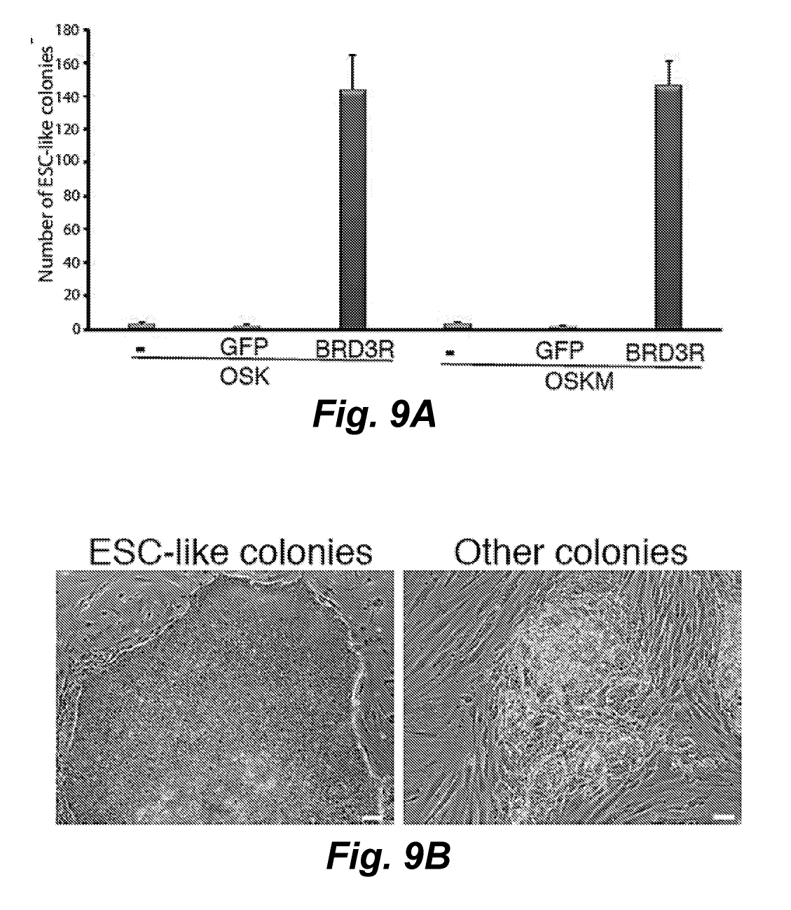

FIG. 1D illustrates the validation of the reprogramming activity of BRD3R with TRA-1-60 as a marker (n=3; ***, p<0.001).

FIG. 1E illustrates the representative images of TRA-1-60 staining for the reprogramming dishes in FIG. 1D. 4F: OCT4, SOX2, KLF4 and MYC; 3F: OCT4, SOX2 and KLF4

FIGS. 2A-2G illustrate that BRD3R regulates mitosis during reprogramming.

FIG. 2A illustrates the percentage of cells in different cell cycle phases on day 6 of reprogramming (n=3; *, p<0.05; **, p<0.01).

FIG. 2B illustrates representative flow-cytometry histograms from experiments in A.

FIG. 2C illustrates the numbers of mitotic cells collected in shake-off experiments of reprogramming cells on day 4 (n=3; *, p<0.05).

FIG. 2D illustrates the percentage of SA-β-galactosidase⁺ cells under different reprogramming conditions (on day 5, n=10; **, p<0.01; ***, p<0.01).

FIG. 2E illustrates representative images of SA-β-galactosidase staining (blue) of reprogramming cells in D. Note that the morphology in BRD3R-transduced cells is dissimilar from that of typical fibroblasts, and resembles those of mitotic cells (small and round) (red star). Bar, 50 μm.

FIG. 2F illustrates confocal images of cells on day 3 of reprogramming transduced with HA-tagged BRD3R plus OSK showing BRD3R association with mitotic chromatin (visualized using HA antibody).

FIG. 2G illustrates the confocal images showing Pol II dissociation from mitotic chromatin in reprogramming cells. Bars in F and G, 10 μm.

FIGS. 3A-3C illustrates that a set of human mitotic genes is upregulated by BRD3R in early stages of reprogramming.

FIG. 3A illustrates the gene counts for the top-21 GO terms for the 335 mapped genes that are upregulated by BRD3R on day 3 of reprogramming (AFC≥1.7, p<0.05). Mitotic GO terms are highlighted in red font.

FIG. 3B illustrates the RT-qPCR verification of 11 mitotic genes randomly selected from the 185 BRD3R-upregulated mitotic genes.

FIG. 3C illustrates the AFC for the 24 mitotic genes listed in Table 2. The red line marks the no-change level. Numbers above bars of each gene are the p values for each AFC. The apparent upregulation of CCNA1 and CDKN1C are indicated in C.

FIGS. 4A-4E illustrate that BRD3R-regulated mitotic genes constitute a PSC fingerprint.

FIG. 4A illustrates the Box plots showing higher expression levels of the 24 BRD3R-regulated mitotic genes in human ESCs and human iPSC compared to 20 human tissues, based on dataset GSE34200.

FIGS. 4B and 4C illustrate the fold enrichment of the 24 BRD3R-regulated mitotic genes in H9 (FIG. 4B) and iPSC (FIG. 4C) compared to 3 BJ samples and one keratinocyte (based on RNA sequencing). The line marks the no-change level.

FIGS. 4D and 4E illustrate RT-qPCR verification of the up-regulation of the BRD3R-regulated mitotic genes in ESC (4D) and iPSC (4E) (11 randomly selected genes).

FIG. 5 illustrates a model for BRD3R modulation of reprogramming process. Over-expression of BRD3R upregulates a great number of mitotic genes, and results in increased numbers of mitotic cells privileged for reprogramming, which contributes to the enhanced reprogramming efficiency. BRD3R also facilitates the resetting of the PSC cell cycle structure. The gradient box indicates lower expressions of a set of mitotic genes in the starting fibroblasts; the gradient box designates the elevated expressions of the mitotic genes in BRD3R-expressing reprogramming cells. Arrows indicate a change from one state to another; black arrows represent positive regulations.

FIGS. 6A-6K illustrate the establishment of a sensitive reprogramming protocol capable of simultaneous evaluation of 22×n genes (plus two controls for each 22 genes) for their human reprogramming activities.

FIG. 6A illustrates a map of a modified lentiviral reprogramming vector with labels of the major vector components

FIG. 6B illustrates a map of a modified lentiviral reprogramming vector pLVH-EF1a.AcGFP-P2A-hOct4 (KH162) for the expression of Oct4, with labels of the major vector components. Nucleotide sequence is according to SEQ ID NO: 58.

FIG. 6C illustrates a map of a modified lentiviral reprogramming vector pLVH-EF1a.AcGFP-P2A-hSox2 (KH163) for the expression of Sox2, with labels of the major vector components. Nucleotide sequence is according to SEQ ID NO: 59.

FIG. 6D illustrates a map of a modified lentiviral reprogramming vector pLVH-EF1a.AcGFP-P2A-hKlf4 (KH164) for the expression of Klf4, with labels of the major vector components. Nucleotide sequence is according to SEQ ID NO: 60.

FIG. 6E illustrates a map of a modified lentiviral reprogramming vector pLVH-EF1a-attB1-BRD3-attB2 (KH226) for the expression of BRD3, with labels of the major vector components. Nucleotide sequence is according to SEQ ID NO: 61.

FIG. 6F illustrates the efficient transduction of BJ cells in one well of a 24-well plate with 250 μl of GFP viral supernatant packaged in one well of a 6-well plate. GFP lentiviral construct is shown as in A without the second transgene after P2A.

FIG. 6G illustrates the flow-cytometry histogram of cells in B. Green is the transduced cells, and red is the control of untransduced cells.

FIG. 6H illustrates the RT-qPCR estimations of mRNA levels of the two randomly selected kinase genes from the library delivered into BJ cells with 250 μl of transgene viral supernatant packaged in a well of a 6-well plate (n=3, mean±SD).

FIG. 6I illustrates a sensitive demonstration of reprogramming promoting activities of the two established minor reprogramming factors NANOG and LIN28 by an optimized small-vessel reprogramming protocol (n=3, mean±SD).

FIG. 6J illustrates representative images of reprogramming dishes stained for ALP from experiments in E.

FIG. 6K illustrates a map of a lentiviral destination vector for Gateway cloning of cDNA library, with labels of the major vector components.

FIG. 7 illustrates a comparison of cell morphology and colony morphology of BRD3R reprogramming with control reprogramming at different time of reprogramming. BJ cells were treated with reprogramming factors as indicated. Upper panel, on day 5, BRD3R reprogramming generated a lot of small and round cells (green stars) distinct from the typical elongated fibroblast cells (black triangles), with less senescence cells (red polygons in control reprogramming). At this stage, the cell density in each treatment displays no difference. Middle panel, at mid stage of reprogramming (day 15), BRD3R dishes contain a lot of colonies while control dishes present much less colonies, and the colony size in control is smaller. At this stage, the cell densities in surrounding areas are similar, and therefore cells in BRD3R colonies contribute to the increased numbers of cells as seen in CyQuant cell proliferation assay (fig. S10). Lower panel, BRD3R dishes present much more colonies, and the colonies are more similar to that of the established iPSC/hESC with clear border and smooth colony surface. The cells in BRD3R colonies are more homogenous. Bar in upper panel, 100 μm; bar in middle and lower panels, 200 μm.

FIGS. 8A-8E illustrate that BRD3R speeds up reprogramming kinetics.

FIG. 8A illustrates representative images showing early appearance of TRA-1-60⁺ clusters in BRD3R reprogramming. TRA-1-60⁺ clusters are frequently seen as early as day 6 in BRD3R reprogramming (arrowhead), but it is generally not seen on day 8 for control reprogramming. In addition, a lot of small round cells appear in BRD3R reprogramming (red star), but this is less frequent in control.

FIG. 8B illustrates the numbers of TRA-1-60⁺ colonies on day 15 and 25 of reprogramming (n=3; mean±SD; ***, p<0.001).

FIG. 8C illustrates representative images of reprogramming dishes stained for TRA-1-60 on day 15 and day 25. Note the larger colonies in BRD3R reprogramming.

FIG. 8D illustrates the numbers of TRA-1-60⁺ clusters on day 10 of reprogramming (n=3; mean±SD; ***, p<0.001).

FIG. 8E illustrates representative images of TRA-1-60⁺ clusters from experiments in D. Note the larger TRA-1-60⁺ clusters in BRD3R reprogramming.

FIGS. 9A and 9B illustrate that BRD3R reprogramming generates significantly more high-quality colonies than control both in the context of 3F and 4F.

FIG. 9A illustrates the quantification of ESC-like colonies (n=3; mean±SD).

FIG. 9B illustrates representative images of ESC-like colonies (left) and low-quality colonies (right) on which the quantification in A was based. ESC-like colony has clear border with smooth colony surface and contains homogenous cells, whereas the low-quality colonies have ragged colony border and surface, and contain heterogeneous cells. Bars in B represent 100 μm.

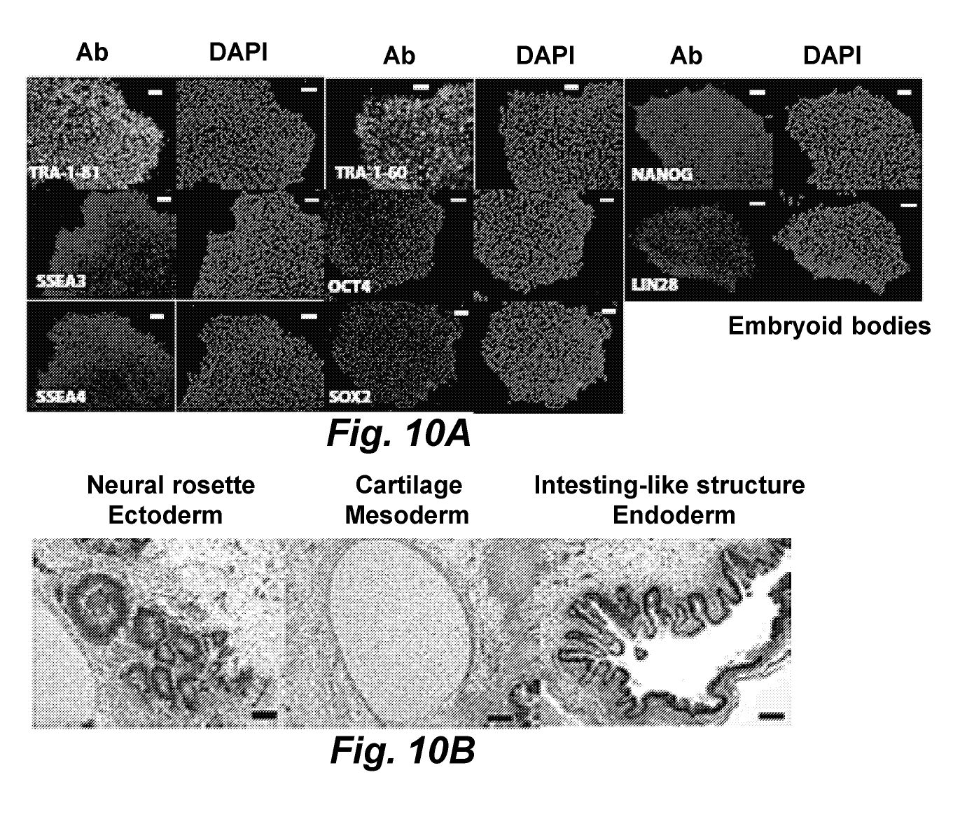

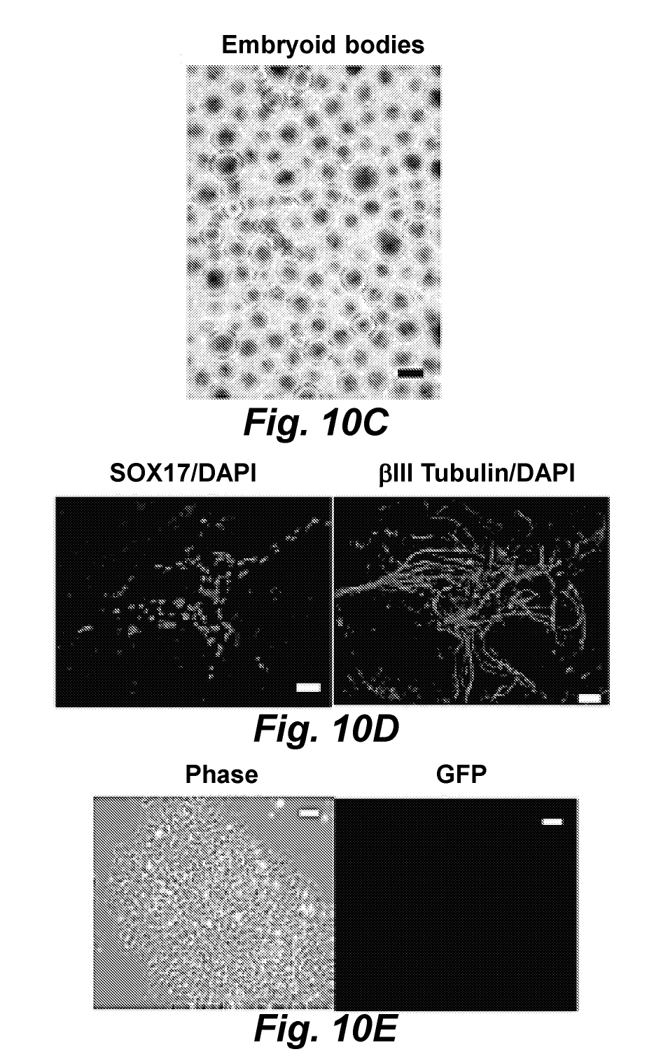

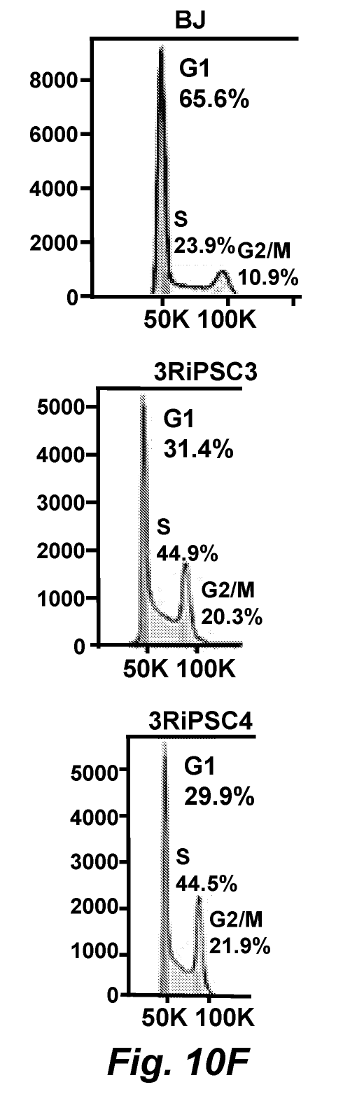

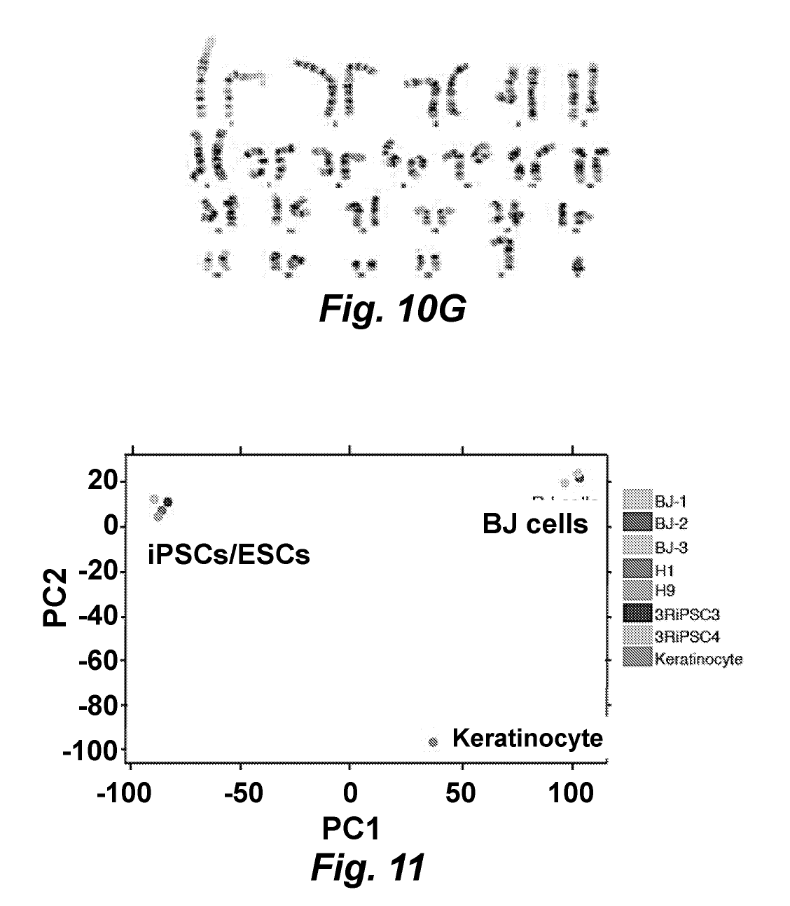

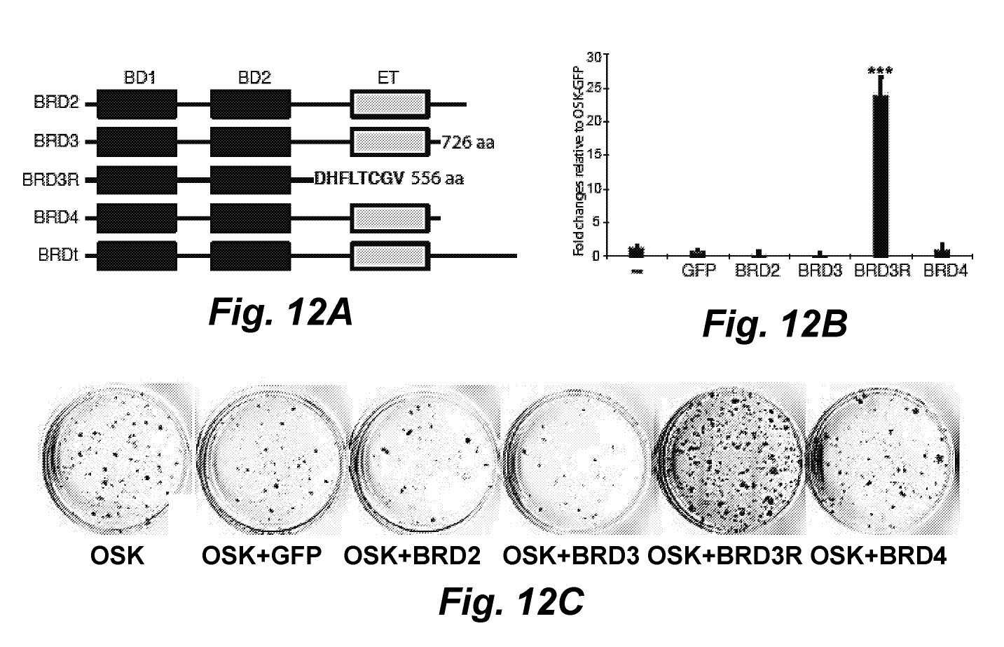

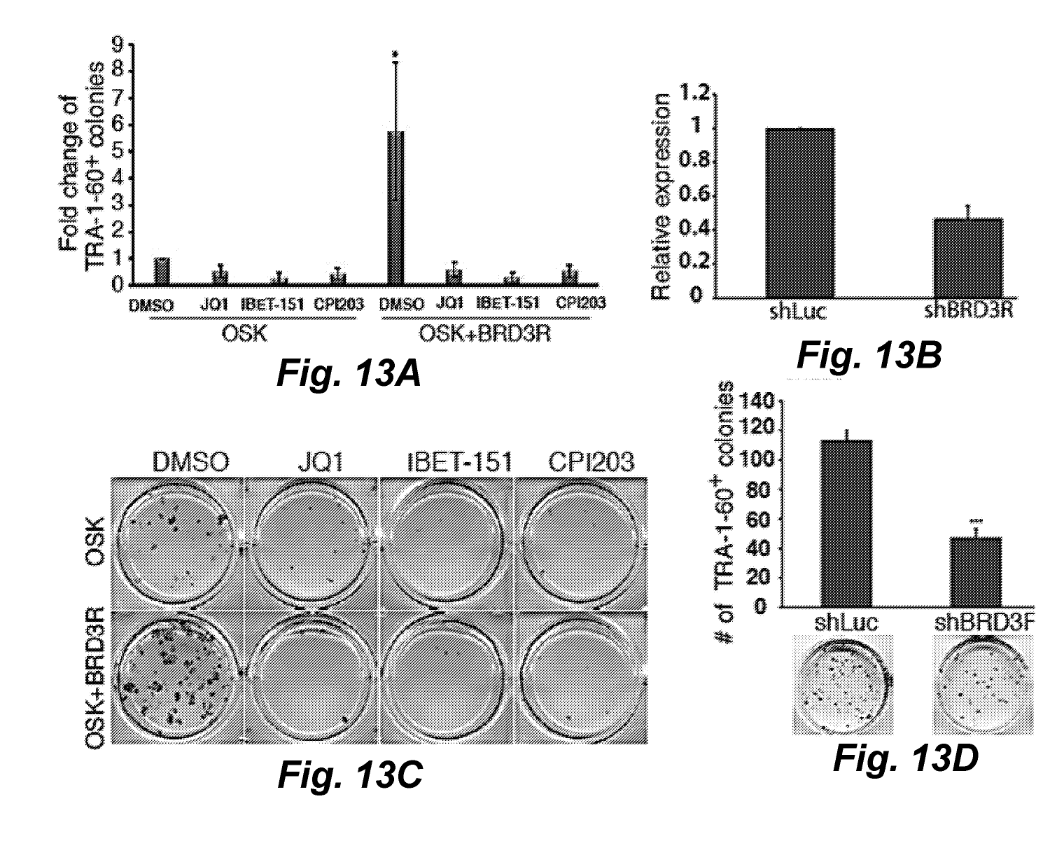

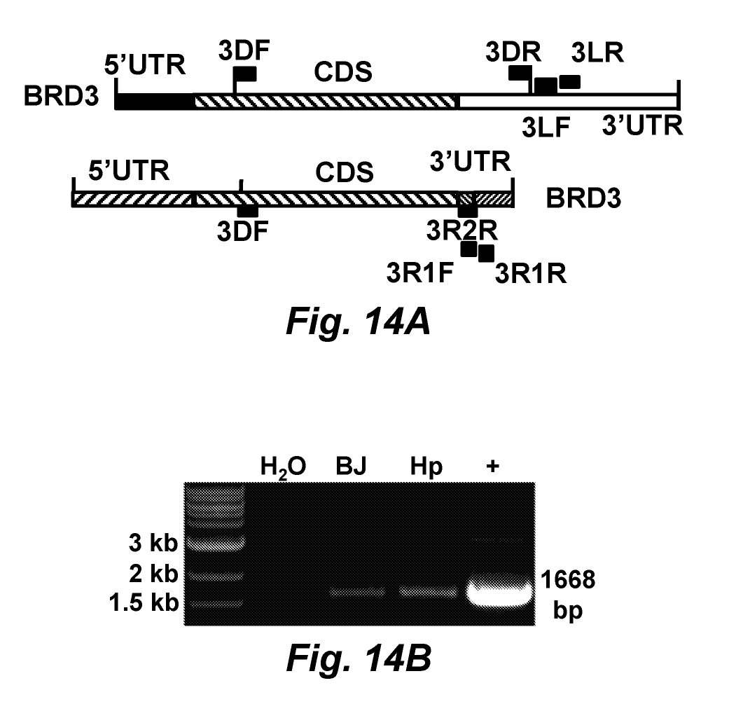

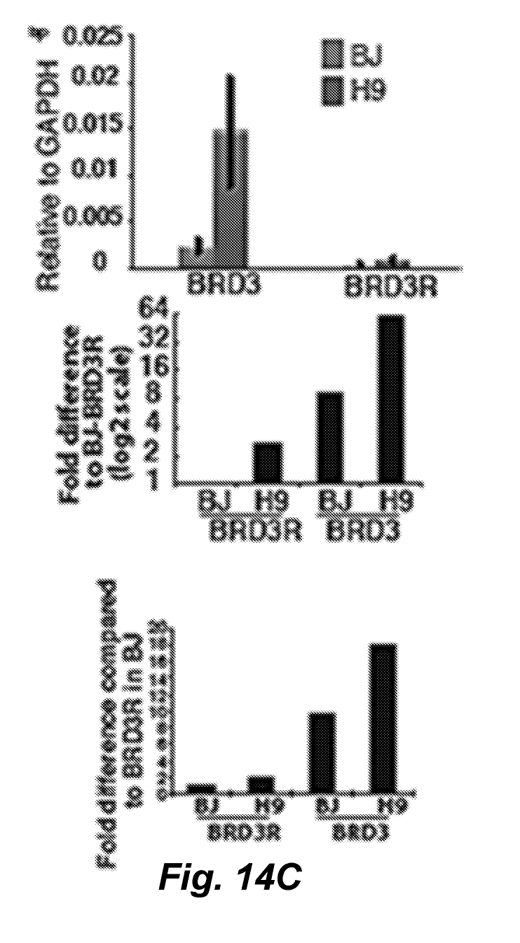

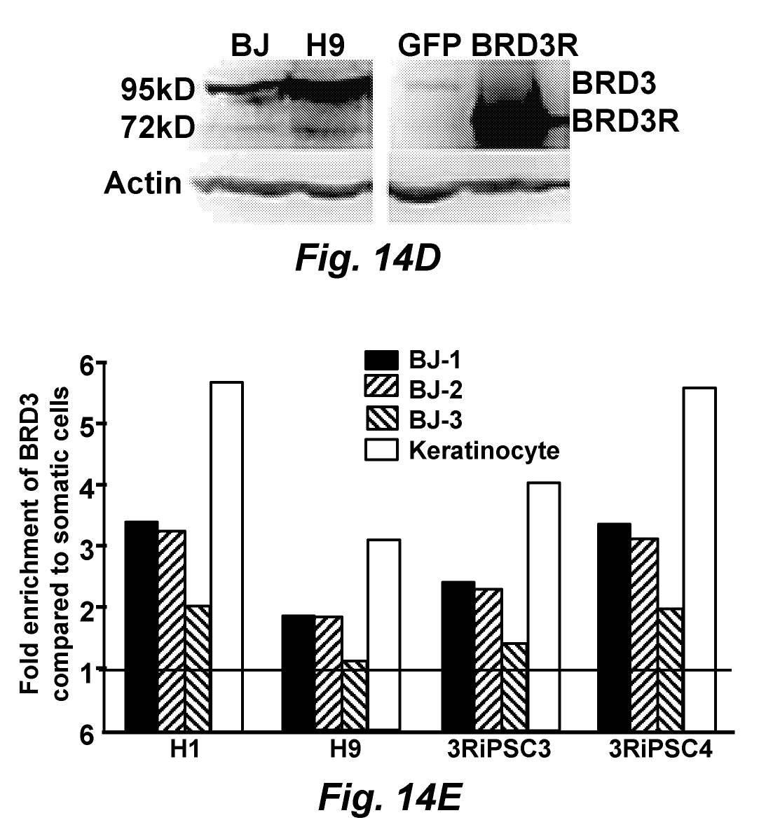

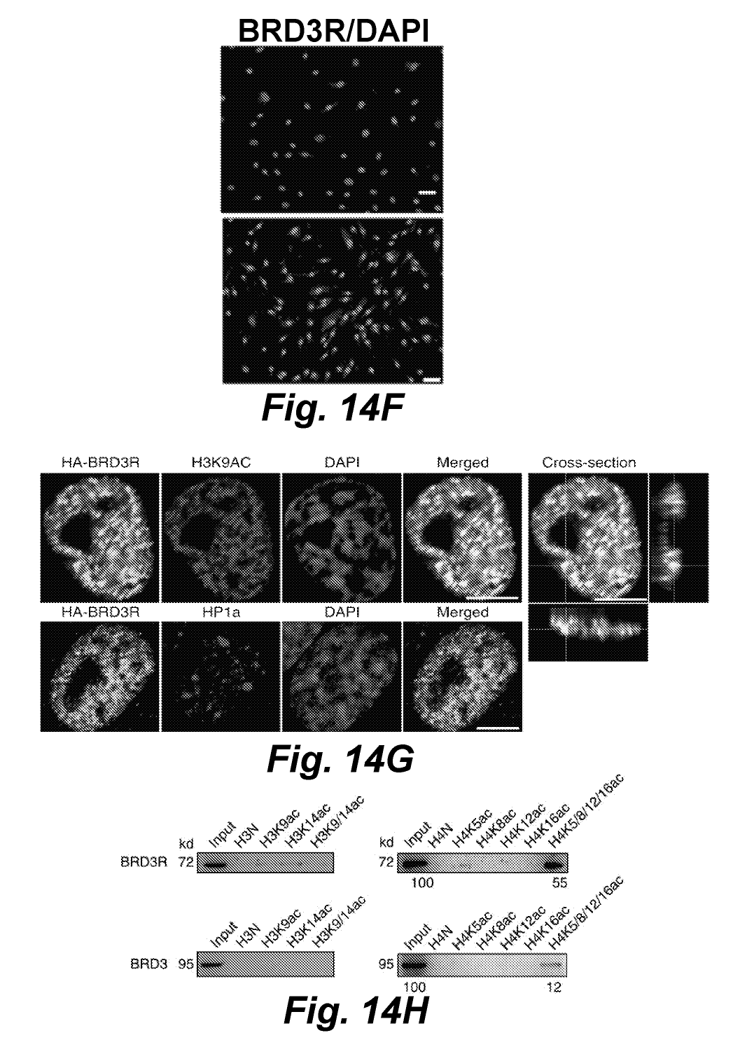

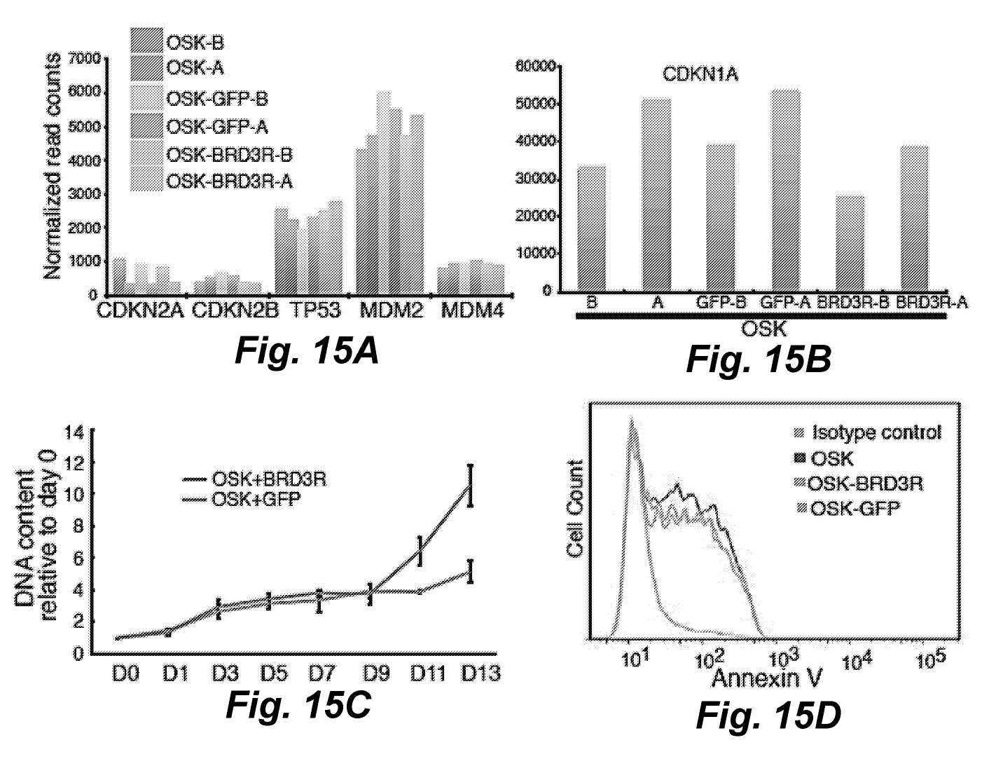

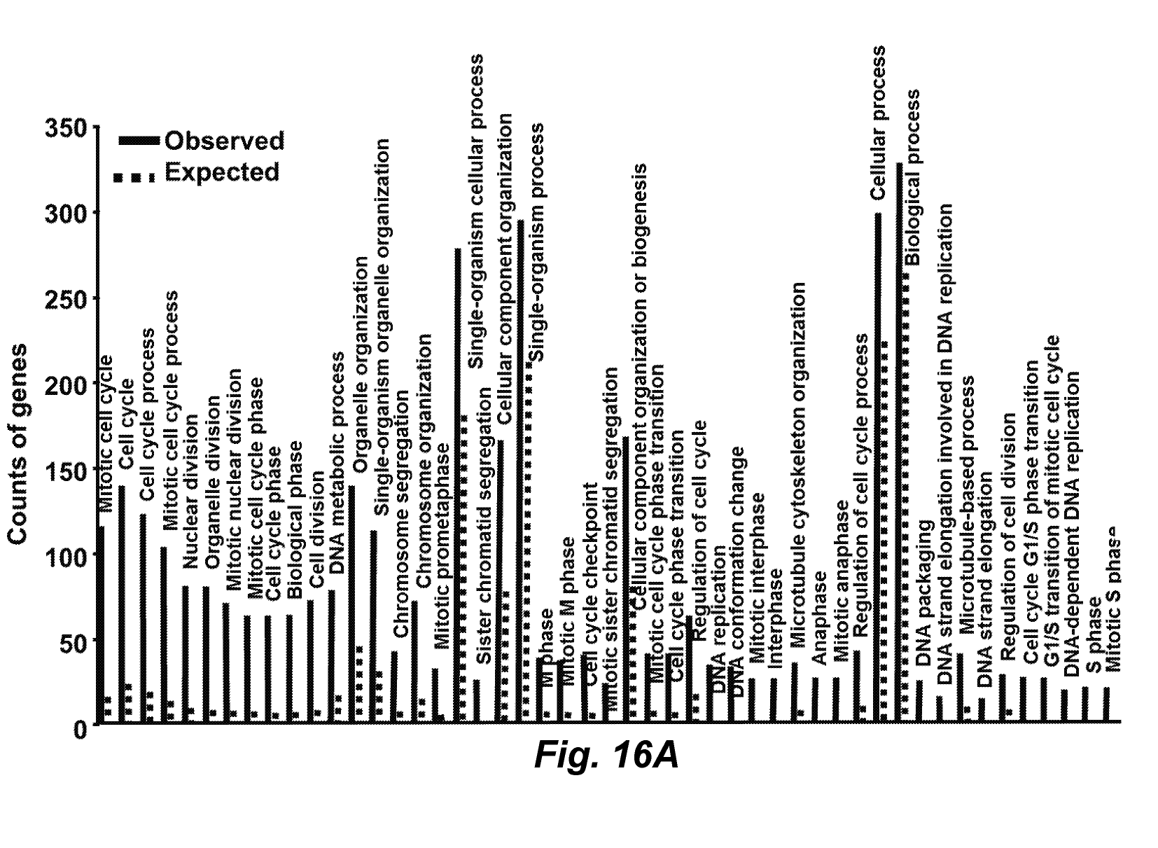

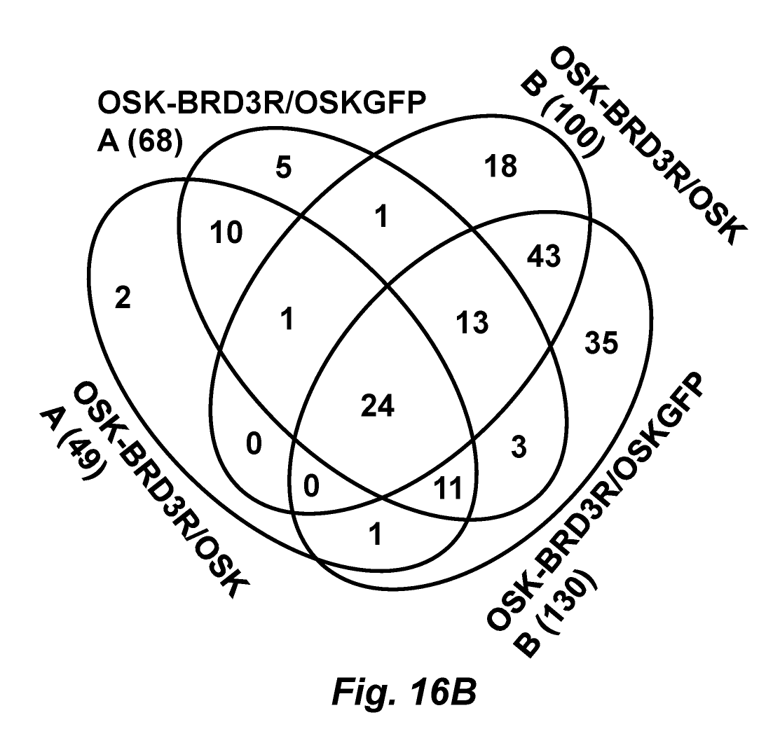

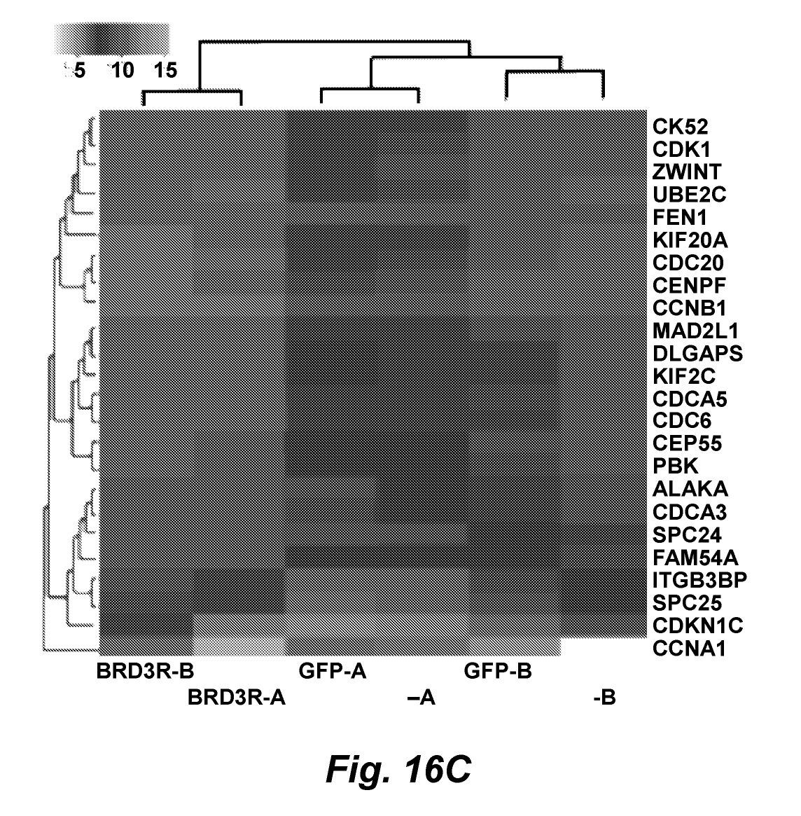

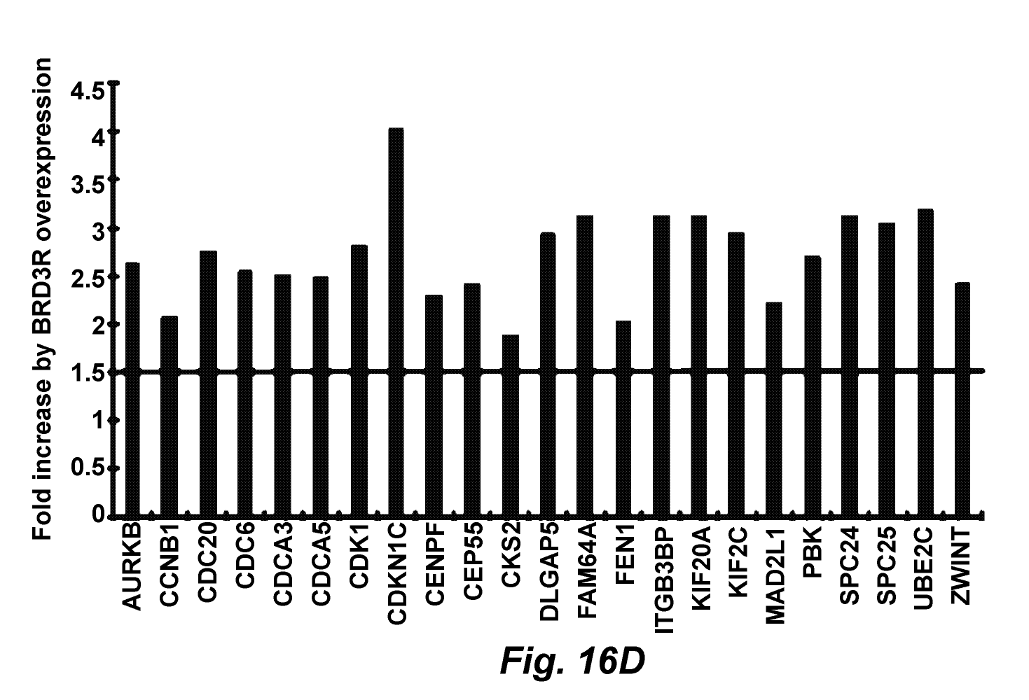

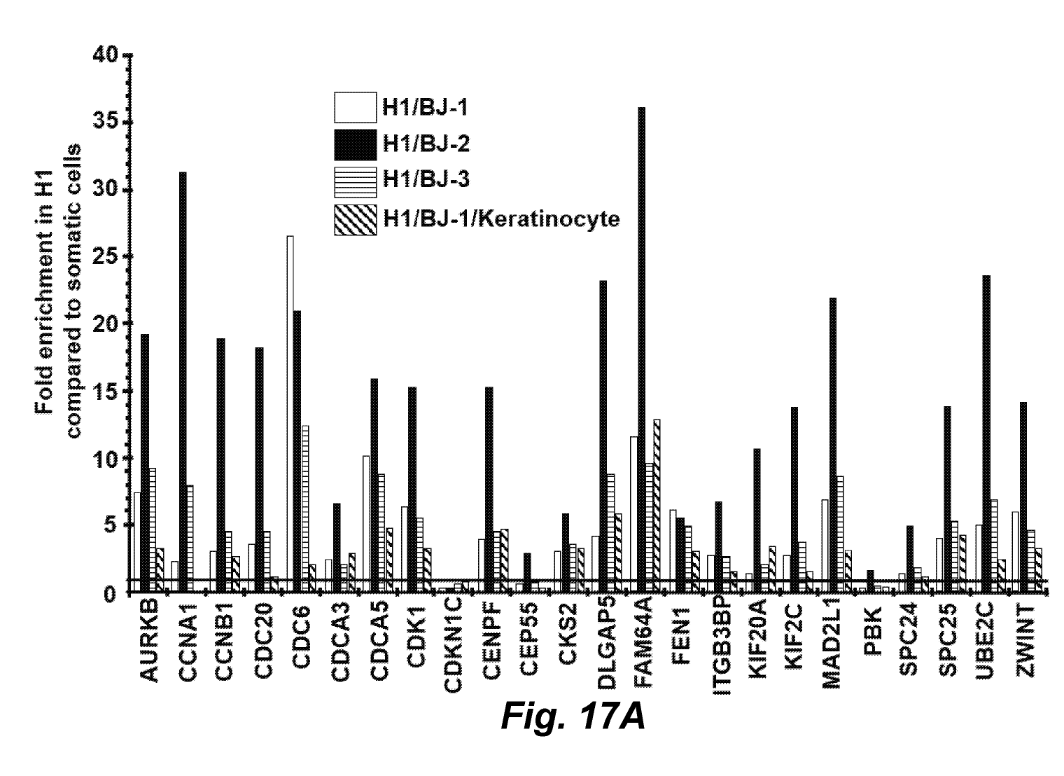

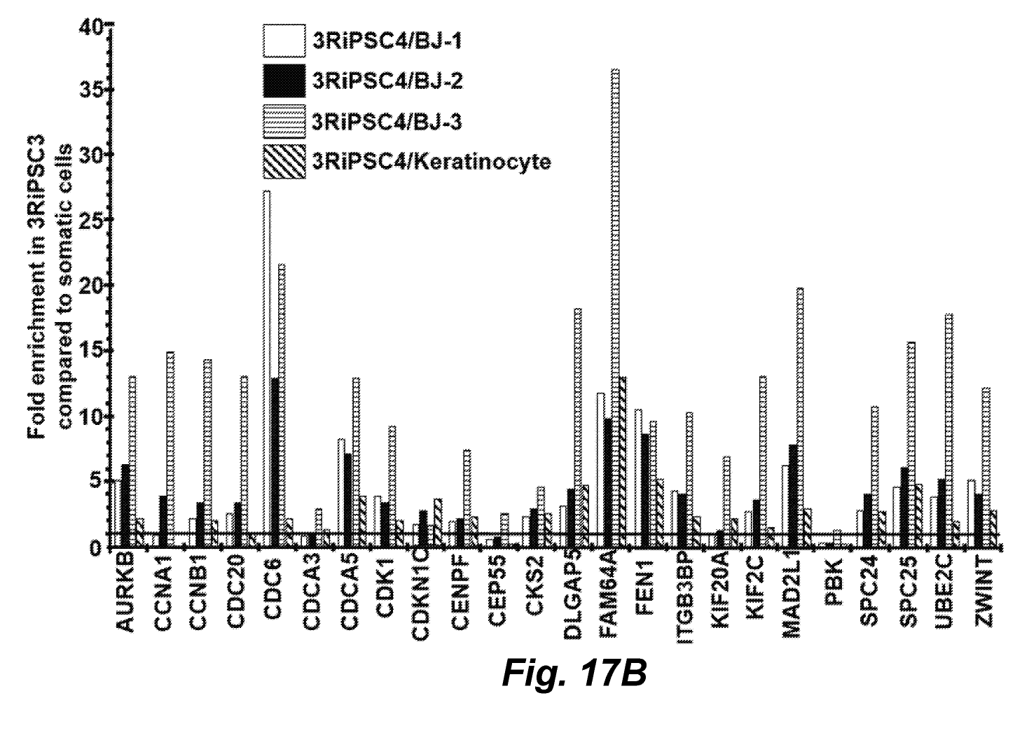

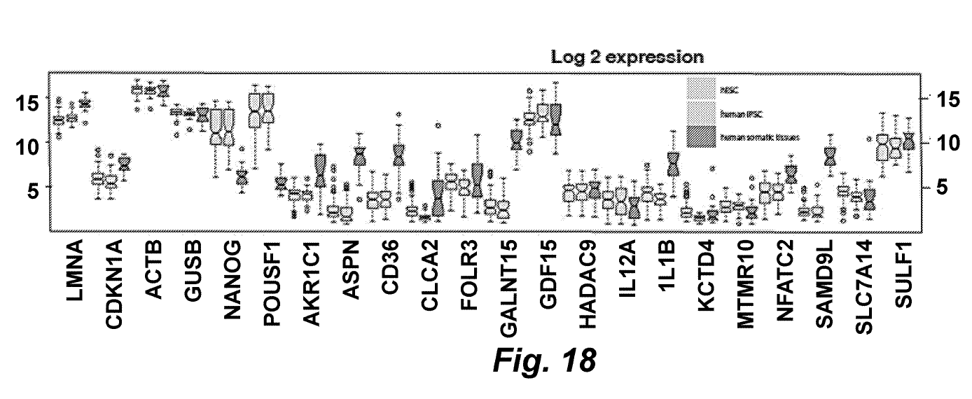

FIGS. 10A-10G illustrate that BRD3R reprogrammed iPSCs are pluripotent.

FIG. 10A illustrates representative images for immunostaining of BRD3R iPSCs (3RiPSC2) for the established pluripotent surface markers TRA-1-81, TRA-1-60, SSEA3 and SSEA4, and for the pluripotent factors OCT4, SOX2, NANOG, and LIN28. Nuclei are visualized with DAPI staining.

FIG. 10B illustrates the H&E staining of representative teratoma sections demonstrating a capacity of BRD3R iPSCs (3RiPSC2) to generate cells representing all three germ layers.

FIG. 10C illustrates uniform embryoid bodies generated from BRD3R iPSCs (3RiPSC2).

FIG. 10D illustrates the immunostaining of differentiated BRD3R iPSCs (3RiPSC2) demonstrating a capacity to form endoderm (SOX17) and ectoderm (beta-III tubulin).

FIG. 10E illustrates the silencing of reprogramming factors in the established BRD3R iPSCs (3RiPSC2) as indicated by the absence of GFP expression, which is co-expressed with the reprogramming factors mediated by a 2A peptide.

FIG. 10F illustrates flow cytometry histograms demonstrating successful resetting of the typical pluripotent cell cycle structure in the established BRD3R iPSCs. Note the shortened G1 phase in BRD3R iPSCs.

FIG. 10G illustrates the normal karyotype of the established BRD3R iPSCs ((3RiPSC4). Note the male karyotype in agreement with its origin of foreskin fibroblasts (BJ cells). Bars in A, D and E, 50 μm; bars in C, 200 μm; bar in B, 100 μm.

FIG. 11 is a graph illustrating the close similarity of BRD3R iPSCs to human embryonic stem cells as demonstrated by principal component analysis (PCA). Four types of cells analyzed are human embryonic stem cells (H1 and H9), BRD3R human iPSCs (3RiPSC3 and 3RiPSC4), human fibroblasts (BJ cells, three RNA sequencing samples) and an isolate of human keratinocyte. PCA analysis was based on RNA sequencing data.

FIGS. 12A-12C illustrate that other human BET members do not exhibit reprogramming activities.

FIG. 12A illustrates the domain structure of human BET family members (not to scale). Black box, bromodomain; grey box, ET domain; the single-letter sequence at the C-terminus of BRD3R is the unique tail of BRD3R as a result of alternative splicing.

FIG. 12B illustrates the fold change in numbers of TRA-1-60⁺ colonies for reprogramming with different human BET members when used with 3F in reprogramming of human BJ cells, as compared to OSK-GFP control (n=3; mean±SD; ***, p<0.001). BRDT was not tested due to its restricted expression.

FIG. 12C illustrates representative images of TRA-1-60 staining for the reprogramming dishes of experiments in panel B.

FIGS. 13A-13D illustrate that BET inhibition impairs human reprogramming.

FIG. 13A illustrates the fold changes of TRA-1-60⁺ colonies for reprogramming treated for 5-7 days with the BET inhibitors JQ1 (500 nM), I-BET-151 (10 μM), and CPI-203 (1 μM) and with DMSO as control.

FIG. 13B illustrates representative reprogramming dishes stained for TRA-1-60.

FIG. 13C illustrates the significant knockdown of BRD3R mRNA with BRD3R-specific shRNA.

FIG. 13D illustrates the knockdown of BRD3R impairs human reprogramming. Images beneath each bar are representative reprogramming dishes stained for TRA-1-60. Data are presented as mean±SD (n=3). *, p<0.05; ***, p<0.001.

FIGS. 14A-14E illustrate that BRD3R/BRD3 are enriched in PSCs.

FIG. 14A illustrates the cDNA structure and primer location of BRD3R in comparison with its long isoform BRD3. Red box, identical cDNA regions. Primer locations are indicated by black boxes with primer name by the side. F in primer name, forward primers; R in primer name, reverse primers. Primer sequences are given in Table 3.

FIG. 14B illustrates the semi-quantitative RT-PCR with BRD3R-specific primers, demonstrating a higher expression level in hESCs (H9) than in somatic cells (BJ). Upper panel, gel image of the RT-PCR; Lower panel, quantification of the amplified cDNA in the gel above. +, positive control with BRD3R plasmid as PCR template; H₂O, control without template.

FIG. 14C illustrates the RT-qPCR quantification of BRD3R/BRD3 expression in H9 cells in comparison with that in BJ cells. Upper panel, relative expression to GAPDH; Lower panel, fold difference compared to level of BRD3R in BJ cells, calculated from upper panel (in triplicates).

FIG. 14D illustrates the Western analysis of BRD3R/BRD3 protein. Left, protein samples from naïve BJ and H9 cells; Middle, protein samples from BJ cells transduced with GFP (left) and BRD3R (right) lentiviruses; the lower parts in left and middle panels are beta-actin loading control. Right panel, quantification of the protein level from the left panel, relative to protein level of BRD3R in BJ cells (lower band in the BJ lane).

FIG. 14E illustrates the fold enrichment of BRD3R/BRD3 mRNA in human PSCs compared to somatic cells calculated from RNA sequencing data of three BJ RNA samples, one human keratinocyte sample, two hESC and two human iPSC lines established using BDR3R. The red line indicates the level of a fold change of one (no change).

FIG. 14F illustrates that BRD3R localizes in the nucleus. Anti-BRD3 antibody was used. Upper panel, BJ cells overexpressing GFP control; lower panel, BJ cells overexpressing BRD3R. Nuclei were visualized using DAPI. Scale bar, 50 μm.

FIG. 14G illustrates confocal images showing BRD3R localization in distinct regions of chromatin from those of the heterochromatin marker HP1α, and co-localization with euchromatin marker H3K9Ac. Chromatin was visualized using DAPI. The y-axis and z-axis cross sections at a co-localization site are shown along with the x-axis section at the end of the upper row, indicating BRD3R co-localization with H3K9Ac in the space (crosses). Scale bar, 5 μm.

FIG. 14H illustrates peptide pull-down experiments showing differential binding to acetylated histones by BRD3 isoforms.

FIGS. 15A-15D illustrate that BRD3R does not promote reprogramming by regulating p53-p21 pathway.

FIGS. 15A and 15B illustrate normalized read counts of RNA sequencing data for members of ARF-p53 DNA surveillance pathway. RNA samples were prepared from day-3 reprogramming BJ cells transduced with viral particles of reprogramming factors as indicated.

FIG. 15C illustrates the comparison of cell proliferation between BRD3R and control reprogramming, showing similar growth rate in early stages of reprogramming (before day 9).

FIG. 15D illustrates the flow cytometry histograms showing similar apoptosis between BRD3R and control reprogramming (day 5).

FIGS. 16A-16E illustrate that BRD3R upregulates a large set of mitotic genes during early reprogramming.

FIG. 16A illustrates the tally of genes for the top 49 GO terms listed in A.

FIG. 16B illustrates the Venn diagram showing overlapping mitotic genes from the four lists of mitotic genes upregulated (>1.5×, p<0.05) by BRD3R overexpression on day 3 of reprogramming among independent experiments. Total numbers of the upregulated mitotic genes are given in brackets for each comparison.

FIG. 16C illustrates the heat map of expression levels as determined by RNA sequencing for the 24 consistently upregulated mitotic genes (as identified in C) by BRD3R overexpression on day 3 of reprogramming in the context of OSK reprogramming.

FIG. 16D illustrates a bar diagram showing representative individual fold increases of the 24 consistently up-regulated mitotic genes by BRD3R overexpression on day 3 of reprogramming (comparison OSK-BRD3R-B/OSK-GFP-B). The red line marks the level of 1.5-fold increase (p<0.05). CCNA1 is not listed due to scale inconvenience.

FIGS. 17A and 17B illustrate that BRD3R-regulated mitotic genes are enriched in human embryonic stem cells and human iPSCs.

FIG. 17A illustrates the fold enrichment of the 24 BRD3R-regulated mitotic genes in H1 cells compared to BJ human fibroblast and human keratinocyte based on RNA sequencing data. Line marks the no-change level (1 fold change).

FIG. 17B illustrates the fold enrichment of the 24 BRD3R-regulated mitotic genes in BRD3R iPSC3 cells (3RiPSC3) compared to BJ human fibroblasts and human keratinocyte, based on RNA sequencing data. Line marks the no-change level (1 fold change).

FIG. 18 illustrates a boxplot showing that only five of the 17 fibroblast-enriched genes are enriched in other somatic cells compared to PSCs. The boxplot is based on dataset GSE34200 (log 2 expression), which is microarray dataset for the 21 human embryonic stem cells (132 microarray samples), 8 human iPSCs (46 microarray samples) and 20 human tissues. The gene PTCHD4 is not available in the dataset GSE34200. Genes with higher expression in human somatic tissues are highlighted. The five genes that are also enriched in human keratinocyte (based on RNA sequencing, data not shown) are in boldface and underlined.

### DESCRIPTION OF THE DISCLOSURE

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

As used herein, the following terms have the meanings ascribed to them unless specified otherwise. In this disclosure, “comprises,” “comprising,” “containing” and “having” and the like can have the meaning ascribed to them in U.S. patent law and can mean “includes,” “including,” and the like; “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially of” or “consists essentially” or the like, when applied to methods and compositions encompassed by the present disclosure have the meaning ascribed in U.S. patent law, allowing for the presence of more than that which is recited so long as basic or novel characteristics of that which is recited is not changed by the presence of more than that which is recited, but excludes prior art embodiments.

### Abbreviations

iPSC, induced Pluripotent Stem Cell; SCNT, somatic cell nuclear transfer; PSC, pluripotent stem cell; ARF, alternate reading frame tumor suppressor; BRD3, bromodomain-containing 3; BRD3R, BRD3 with reprogramming activity; GFP, green fluorescent protein; ALP+, alkaline phosphatase expressing.

### Definitions

The term “added co-transcriptionally” as used herein refers to the addition of a feature, e.g., a 5′ diguanosine cap or other modified nucleoside, to a synthetic, modified RNA during transcription of the RNA molecule (i.e., the modified RNA is not fully transcribed prior to the addition of the 5′ cap).

The term “c-myc” as used herein refers to a transcription factor that is well known by those skilled in the art. It regulates the expression of many genes and recruits histone transacetylase. Its mutations are related to many cancers.

The term “cell surface marker” as used herein refers to a protein expressed on the surface of a cell, which is detectable via specific antibodies. Cell surface markers that are useful in the methods of the disclosure include, but are not limited to, the CD (clusters of differentiation) antigens CD1a, CD2, CD3, CD5, CD7, CD8, CD10, CD13, CD14, CD16, CD19, CD29, CD31, CD33, CD34, CD35, CD38, CD41, CD45, CD56, CD71, CD73, CD90, CD105, CD115, CD117, CD124, CD127, CD130, CD138, CD144, CD166, HLA-A, HLA-B, HLA-C, HLA-DR, VEGF receptor 1(VEGF-R1), VEGF receptor-2 (VEGF-R2), and glycophorin A. By “intracellular marker” is intended expression of a gene or gene product such as an enzyme that is detectable. For example, aldehyde dehydrogenase (ALDH) is an intracellular enzyme that is expressed in most hematopoietic stem cells. It can be detected via flow cytometry by using fluorescent substrates.

The term “cell-type specific polypeptide” as used herein refers to a polypeptide that is expressed in a cell having a particular phenotype (e.g., a muscle cell, a pancreatic β-cell) but is not generally expressed in other cell types with different phenotypes. As but one example, MyoD is expressed specifically in muscle cells but not in non-muscle cells, thus MyoD is a cell-type specific polypeptide.

The term “contacting” or “contact” as used herein in connection with contacting a cell with one or more synthetic, modified RNAs as described herein, includes subjecting a cell to a culture medium which comprises one or more synthetic, modified RNAs at least one time, or a plurality of times, or to a method whereby such a synthetic, modified RNA is forced to contact a cell at least one time, or a plurality of times, i.e., a transfection system. Where such a cell is in vivo, contacting the cell with a synthetic, modified RNA includes administering the synthetic, modified RNA in a composition, such as a pharmaceutical composition, to a subject via an appropriate administration route, such that the compound contacts the cell in vivo.

The terms “developmental potential” or “developmental potency” as used herein refer to the total of all developmental cell fates or cell types that can be achieved by a cell upon differentiation. Thus, a cell with greater or higher developmental potential can differentiate into a greater variety of different cell types than a cell having a lower or decreased developmental potential. The developmental potential of a cell can range from the highest developmental potential of a totipotent cell, which, in addition to being able to give rise to all the cells of an organism, can give rise to extra-embryonic tissues; to a “unipotent cell,” which has the capacity to differentiate into only one type of tissue or cell type, but has the property of self-renewal, as described herein; to a “terminally differentiated cell,” which has the lowest developmental potential. A cell with “parental developmental potential” refers to a cell having the developmental potential of the parent cell that gave rise to it.

The term “developmental potential altering factor,” as used herein refers to a factor such as a protein or RNA, the expression of which alters the developmental potential of a cell, e.g., a somatic cell, to another developmental state, e.g., a pluripotent state. Such an alteration in the developmental potential can be a decrease (i.e., to a more differentiated developmental state) or an increase (i.e., to a less differentiated developmental state). A developmental potential altering factor can be, for example, an RNA or protein product of a gene encoding a reprogramming factor, such as SOX2, an RNA or protein product of a gene encoding a cell-type specific polypeptide transcription factor, such as myoD, a microRNA, a small molecule, and the like.

The terms “differentiate”, or “differentiating” as used herein are relative terms that refer to a developmental process by which a cell has progressed further down a developmental pathway than its immediate precursor cell. Thus in some embodiments, a reprogrammed cell as the term is defined herein, can differentiate to a lineage-restricted precursor cell (such as a mesodermal stem cell), which in turn can differentiate into other types of precursor cells further down the pathway (such as a tissue specific precursor, for example, a cardiomyocyte precursor), and then to an end-stage differentiated cell, which plays a characteristic role in a certain tissue type, and may or may not retain the capacity to proliferate further.

The term “differentiation factor” as used herein refers to a developmental potential altering factor, as that term is defined herein, such as a protein, RNA, or small molecule that induces a cell to differentiate to a desired cell-type, i.e., a differentiation factor reduces the developmental potential of a cell. In some embodiments, a differentiation factor can be a cell-type specific polypeptide, however this is not required. Differentiation to a specific cell type can require simultaneous and/or successive expression of more than one differentiation factor. In some aspects described herein, the developmental potential of a cell or population of cells is first increased via reprogramming or partial reprogramming using synthetic, modified RNAs, as described herein, and then the cell or progeny cells thereof produced by such reprogramming are induced to undergo differentiation by contacting with, or introducing, one or more synthetic, modified RNAs encoding differentiation factors, such that the cell or progeny cells thereof have decreased developmental potential.

The term “embryonic stem cell” as used herein refers to naturally occurring pluripotent stem cells of the inner cell mass of the embryonic blastocyst (see, for e.g., U.S. Pat. Nos. 5,843,780; 6,200,806; 7,029,913; 7,584,479, which are incorporated herein by reference). Such cells can similarly be obtained from the inner cell mass of blastocysts derived from somatic cell nuclear transfer (see, for example, U.S. Pat. Nos. 5,945,577, 5,994,619, 6,235,970, which are incorporated herein by reference). Embryonic stem cells are pluripotent and give rise during development to all derivatives of the three primary germ layers: ectoderm, endoderm and mesoderm. In other words, they can develop into each of the more than 200 cell types of the adult body when given sufficient and necessary stimulation for a specific cell type. They do not contribute to the extra-embryonic membranes or the placenta, i.e., are not totipotent.

The distinguishing characteristics of an embryonic stem cell define an “embryonic stem cell phenotype.” Accordingly, a cell has the phenotype of an embryonic stem cell if it possesses one or more of the unique characteristics of an embryonic stem cell, such that that cell can be distinguished from other cells not having the embryonic stem cell phenotype. Exemplary distinguishing embryonic stem cell phenotype characteristics include, without limitation, expression of specific cell-surface or intracellular markers, including protein and microRNAs, gene expression profiles, methylation profiles, deacetylation profiles, proliferative capacity, differentiation capacity, karyotype, responsiveness to particular culture conditions, and the like. In some embodiments, the determination of whether a cell has an “embryonic stem cell phenotype” is made by comparing one or more characteristics of the cell to one or more characteristics of an embryonic stem cell line cultured within the same laboratory.

The term “exogenous transcription factor” as used herein refers to a transcription factor that is not naturally (i.e., endogenously) expressed in a cell of interest. Thus, an exogenous transcription factor can be expressed from an introduced expression cassette (e.g., under control of a promoter other than a native transcription factor promoter) or can be introduced as a protein from outside the cell. The exogenous transcription factor may comprise an Oct polypeptide (e.g., Oct4), a Klf polypeptide (e.g., Klf4), a Myc polypeptide (e.g., c-Myc), a Sox polypeptide (e.g., Sox2), or any combination thereof.

The term “expression” refers to the cellular processes involved in producing RNA and proteins and as appropriate, secreting proteins, including where applicable, but not limited to, for example, transcription, translation, folding, modification and processing. “Expression products” include RNA transcribed from a gene, and polypeptides obtained by translation of mRNA transcribed from a gene. In some embodiments, an expression product is transcribed from a sequence that does not encode a polypeptide, such as a microRNA.

The term “exogenous” as used herein refers to a nucleic acid (e.g., a synthetic, modified RNA encoding a transcription factor), or a protein (e.g., a transcription factor) that has been introduced by a process involving the hand of man into a biological system such as a cell or organism in which it is not normally found, or in which it is found in lower amounts. A factor (e.g. a synthetic, modified RNA encoding a transcription factor, or a protein, e.g., a polypeptide) is considered exogenous if it is introduced into an immediate precursor cell or a progeny cell that inherits the substance. In contrast, the term “endogenous” refers to a factor or expression product that is native to the biological system or cell (e.g., endogenous expression of a gene, such as, e.g., SOX2 refers to production of a SOX2 polypeptide by the endogenous gene in a cell). In some embodiments, the introduction of one or more exogenous factors to a cell, e.g., a developmental potential altering factor, using the compositions and methods comprising synthetic, modified RNAs described herein, induces endogenous expression in the cell or progeny cell(s) thereof of a factor or gene product necessary for maintenance of the cell or progeny cell(s) thereof in a new developmental potential.

The terms “heterologous sequence” or a “heterologous nucleic acid” as used herein refer to sequences that originate from a source foreign to the particular host cell, or, if from the same source, is modified from its original form. Thus, a heterologous expression cassette in a cell is an expression cassette that is not endogenous to the particular host cell, for example by being linked to nucleotide sequences from an expression vector rather than chromosomal DNA, being linked to a heterologous promoter, being linked to a reporter gene, etc.

The term “histone modification” used herein indicates a variety of modifications to histone, such as acetylation, methylation, demethylation, phosphorylation, adenylation, ubiquitination, and ADP ribosylation. In particular, the histone modification includes the demethylation of histone.

The term “identity” as used herein refers to a relationship between two or more polypeptide sequences, as determined by comparing the sequences. In the art, “identity” also refers to the degree of sequence relatedness between polypeptides as determined by the match between strings of such sequences. “Identity” and “similarity” can be readily calculated by known methods, including, but not limited to, those described in Computational Molecular Biology, Lesk, A. M., Ed., Oxford University Press, NY, 1988; Biocomputing: Informatics and Genome Projects, Smith, D. W., Ed., Academic Press, NY, 1993; Computer Analysis of Sequence Data, Part I, Griffin, A. M. & and Griffin, H. G., Eds., Humana Press, NJ, 1994; Sequence Analysis in Molecular Biology, von Heinje, G., Academic Press, 1987; Sequence Analysis Primer, Gribskov, M. & Devereux, J., Eds., M Stockton Press, NY, 1991; and Carillo & Lipman (1988) SIAM J. Applied Math., 48: 1073.

Preferred methods to determine identity are designed to give the largest match between the sequences tested. Methods to determine identity and similarity are codified in publicly available computer programs. The percent identity between two sequences can be determined by using analysis software (i.e., Sequence Analysis Software Package of the Genetics Computer Group, Madison, Wis.) that incorporates the Needelman & Wunsch ((1970) J. Mol. Biol., 48: 443-453) algorithm (e.g., NBLAST and XBLAST). The default parameters are used to determine the identity for the polypeptides of the present disclosure.

By way of example, a polypeptide sequence may be identical to the reference sequence, that is be 100% identical, or it may include up to a certain integer number of amino acid alterations as compared to the reference sequence such that the percent identity is less than 100%. Such alterations are selected from: at least one amino acid deletion, substitution (including conservative and non-conservative substitution), or insertion, and wherein said alterations may occur at the amino- or carboxy-terminus positions of the reference polypeptide sequence or anywhere between those terminal positions, interspersed either individually among the amino acids in the reference sequence, or in one or more contiguous groups within the reference sequence. The number of amino acid alterations for a given percent identity is determined by multiplying the total number of amino acids in the reference polypeptide by the numerical percent of the respective percent identity (divided by 100) and then subtracting that product from said total number of amino acids in the reference polypeptide.

The term “immediate precursor cell” is used herein to refer to a parental cell from which a daughter cell has arisen by cell division.

The term “induced pluripotent stem cells” as used herein refers to cells having properties similar to those of embryonic stem cells and encompasses undifferentiated cells artificially derived from a non-pluripotent cell, typically an adult somatic cell.

The term “incorporation” used herein indicates a process to introduce exogenous substances (such as nucleic acids or proteins) into cells by, for example, calcium phosphate transfection, virus infection, liposome transfection, electroporation, gene gun or the like. Herein, delivering an exogenous polypeptide into cells may be carried out by various methods, for example, by transporters or transport factors, and preferably, by liposome, bacterial polypeptide fragments or the like (refer to WO2002/079417, the content of which is incorporated herein by reference).

The term “isolated” or “partially purified” as used herein refers, in the case of a nucleic acid or polypeptide, to a nucleic acid or polypeptide separated from at least one other component (e.g., nucleic acid or polypeptide) that is present with the nucleic acid or polypeptide as found in its natural source and/or that would be present with the nucleic acid or polypeptide when expressed by a cell, or secreted in the case of secreted polypeptides. A chemically synthesized nucleic acid or polypeptide or one synthesized using in vitro transcription/translation is considered “isolated”.

The term “isolated cell” as used herein refers to a cell that has been removed from an organism in which it was originally found, or a descendant of such a cell. Optionally the cell has been cultured in vitro, e.g., in the presence of other cells. Optionally, the cell is later introduced into a second organism or re-introduced into the organism from which it (or the cell or population of cells from which it descended) was isolated.

The term “isolated population” with respect to an isolated population of cells as used herein refers to a population of cells that has been removed and separated from a mixed or heterogeneous population of cells. In some embodiments, an isolated population is a “substantially pure” population of cells as compared to the heterogeneous population from which the cells were isolated or enriched. In some embodiments, the isolated population is an isolated population of pluripotent cells which comprise a substantially pure population of pluripotent cells as compared to a heterogeneous population of somatic cells from which the pluripotent cells were derived.

The terms “Kif” and “Klf polypeptide” as used herein refer to any of the naturally-occurring members of the family of Kruppel-like factors (Klfs), zinc-finger proteins that contain amino acid sequences similar to those of the Drosophila embryonic pattern regulator Kruppel, or variants of the naturally-occurring members that maintain transcription factor activity, similar e.g., within at least 50%, 80%, or 90% activity compared to the closest related naturally occurring family member, or polypeptides comprising at least the DNA-binding domain of the naturally occurring family member, and can further comprise a transcriptional activation domain. Exemplary Klf family members include, Klf1, Klf2, Klf3, Klf-4, Klf5, Klf6, Klf7, Klf8, Klf9, Klf10, Klf11, Klf12, Klf13, Klf14, Klf15, Klf16, and Klf17. Klf2 and Klf-4 were found to be factors capable of generating iPS cells in mice, and related genes Klf1 and Klf5 did as well, although with reduced efficiency. In some embodiments, variants have at least 85%, 90%, or 95% amino acid sequence identity across their whole sequence compared to a naturally occurring Klf polypeptide family member such as those listed above or such as listed in Genbank accession number CAX16088 (mouse Klf4) or CAX14962 (human Klf4).

The terms “lineage commitment” and “specification,” as used interchangeably herein, refer to the process a stem cell undergoes in which the stem cell gives rise to a progenitor cell committed to forming a particular limited range of differentiated cell types. Committed progenitor cells are often capable of self-renewal or cell division.

The term “multipotent” when used in reference to a “multipotent cell” refers to a cell that has the developmental potential to differentiate into cells of one or more germ layers, but not all three. Thus, a multipotent cell can also be termed a “partially differentiated cell.” Multipotent cells are well known in the art, and examples of multipotent cells include adult stem cells, such as for example, hematopoietic stem cells and neural stem cells. “Multipotent” indicates that a cell may form many types of cells in a given lineage, but not cells of other lineages. For example, a multipotent hematopoietic cell can form the many different types of blood cells (red, white, platelets, etc.), but it cannot form neurons. Accordingly, the term “multipotency” refers to a state of a cell with a degree of developmental potential that is less than totipotent and pluripotent.

The term “nucleic acid molecule” as used herein refers to DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule can be single-stranded or double-stranded, but advantageously is double-stranded DNA. An “isolated” nucleic acid molecule is one that is separated from other nucleic acid molecules that are present in the natural source of the nucleic acid. A “nucleoside” refers to a base linked to a sugar. The base may be adenine (A), guanine (G) (or its substitute, inosine (I)), cytosine (C), or thymine (T) (or its substitute, uracil (U)). The sugar may be ribose (the sugar of a natural nucleotide in RNA) or 2-deoxyribose (the sugar of a natural nucleotide in DNA). A “nucleotide” refers to a nucleoside linked to a single phosphate group.

The terms “Oct” or “Oct polypeptide” as used herein refer to any of the naturally-occurring members of Octamer family of transcription factors, or variants thereof that maintain transcription factor activity, e.g., within at least 50%, 80%, or 90% activity compared to the closest related naturally occurring family member, or polypeptides comprising at least the DNA-binding domain of the naturally occurring family member, and can further comprise a transcriptional activation domain. Exemplary Oct polypeptides include, Oct-1, Oct-2, Oct-3/4, Oct-6, Oct-7, Oct-8, Oct-9, and Oct-11. For example, Oct3/4 (referred to herein as “Oct4”) contains the POU domain, a 150 amino acid sequence conserved among Pit-1, Oct-1, Oct-2, and uric-86 (Ryan et al., (1997) Genes Dev. 11: 1207-1225. Variants have at least 85%, 90%, or 95% amino acid sequence identity across their whole sequence compared to a naturally occurring Oct polypeptide family member such as those listed above or such as listed in Genbank accession number NP_002692.2 (human Oct4) or NP_038661.1 (mouse Oct4). Oct polypeptides (e.g., Oct3/4) can be from human, mouse, rat, bovine, porcine, or other animals. Generally, the same species of protein will be used with the species of cells being manipulated.

The term “oligonucleotide” as used herein refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides may be chemically synthesized and may be used as primers or probes. Oligonucleotide means any nucleotide of more than 3 bases in length used to facilitate detection or identification of a target nucleic acid, including probes and primers.

The term “operably linked” as used herein refers to an arrangement of elements wherein the components so described are configured so as to perform their usual function. Thus, a given promoter operably linked to a coding sequence is capable of effecting the expression of the coding sequence when the proper enzymes are present. The promoter need not be contiguous with the coding sequence, so long as it functions to direct the expression thereof. Thus, for example, intervening untranslated yet transcribed sequences can be present between the promoter sequence and the coding sequence and the promoter sequence can still be considered “operably linked” to the coding sequence.

The term “pluripotent” as used herein refers to a cell with the developmental potential, under different conditions, to differentiate to cell types characteristic of all three germ cell layers, i.e., endoderm (e.g., gut tissue), mesoderm (including blood, muscle, and vessels), and ectoderm (such as skin and nerve). A pluripotent cell has a lower developmental potential than a totipotent cell. The ability of a cell to differentiate to all three germ layers can be determined using, for example, a nude mouse teratoma formation assay. In some embodiments, pluripotency can also evidenced by the expression of embryonic stem (ES) cell markers, although the preferred test for pluripotency of a cell or population of cells generated using the compositions and methods described herein is the demonstration that a cell has the developmental potential to differentiate into cells of each of the three germ layers. In some embodiments, a pluripotent cell is termed an “undifferentiated cell.” Accordingly, the terms “pluripotency” or a “pluripotent state” as used herein refer to the developmental potential of a cell that provides the ability for the cell to differentiate into all three embryonic germ layers (endoderm, mesoderm and ectoderm). Those of skill in the art are aware of the embryonic germ layer or lineage that gives rise to a given cell type. A cell in a pluripotent state typically has the potential to divide in vitro for a long period of time, e.g., greater than one year or more than 30 passages.

Pluripotent stem cell characteristics distinguish pluripotent stem cells from other cells. The ability to give rise to progeny that can undergo differentiation, under the appropriate conditions, into cell types that collectively demonstrate characteristics associated with cell lineages from all of the three germinal layers (endoderm, mesoderm, and ectoderm) is a pluripotent stem cell characteristic. Expression or non-expression of certain combinations of molecular markers are also pluripotent stem cell characteristics. For example, human pluripotent stem cells express at least one, two, or three, and optionally all, of the markers from the following non-limiting list: SSEA-3, SSEA-4, TRA-1-60, TRA-1-81, TRA-2-49/6E, ALP, Sox2, E-cadherin, UTF-1, Oct4, Rex1, and Nanog. Cell morphologies associated with pluripotent stem cells are also pluripotent stem cell characteristics.

The term “polypeptide” as used herein refers to a polymer of amino acids comprising at least 2 amino acids (e.g., at least 5, at least 10, at least 20, at least 30, at least 40, at least 50, at least 60, at least 70, at least 80, at least 90, at least 100, at least 125, at least 150, at least 175, at least 200, at least 225, at least 250, at least 275, at least 300, at least 350, at least 400, at least 450, at least 500, at least 600, at least 700, at least 800, at least 900, at least 1000, at least 2000, at least 3000, at least 4000, at least 5000, at least 6000, at least 7000, at least 8000, at least 9000, at least 10,000 amino acids or more). The terms “protein” and “polypeptide” are used interchangeably herein. The term “peptide” as used herein refers to a relatively short polypeptide, typically between about 2 and 60 amino acids in length.

The term “progenitor cell” is used herein to refer to cells that have greater developmental potential, i.e., a cellular phenotype that is more primitive (e.g., is at an earlier step along a developmental pathway or progression) relative to a cell which it can give rise to by differentiation. Often, progenitor cells have significant or very high proliferative potential. Progenitor cells can give rise to multiple distinct cells having lower developmental potential, i.e., differentiated cell types, or to a single differentiated cell type, depending on the developmental pathway and on the environment in which the cells develop and differentiate.

The terms “proliferation” and “expansion” as used herein interchangeably refer to an increase in the number of cells of the same type by division.

The term “recombinant” as used herein refers to a polynucleotide of genomic, cDNA, semisynthetic, or synthetic origin which, by virtue of its origin or manipulation (1) is not associated with all or a portion of the polynucleotide with which it is associated in nature; and/or (2) is linked to a polynucleotide other than that to which it is linked in nature. The term “recombinant” as used with respect to a protein or polypeptide means a polypeptide produced by expression of a recombinant polynucleotide. “Recombinant host cells,” “host cells,” “cells,” “cell lines,” “cell cultures,” and other such terms denoting eukaryotic cell lines cultured as unicellular entities, are used interchangeably and refer to cells which can be, or have been, used as recipients for recombinant vectors or other transfer DNA, and include the progeny of the original cell which has been transfected. It is understood that the progeny of a single parental cell may not necessarily be completely identical in morphology or in genomic or total DNA complement to the original parent, due to accidental or deliberate mutation. Progeny of the parental cell which are sufficiently similar to the parent to be characterized by the relevant property, such as the presence of a nucleotide sequence encoding a desired peptide, are included in the progeny intended by this definition and are covered by the above terms. Techniques for determining amino acid sequence “similarity” are well known in the art.

The term “reprogramming” as used herein refers to a process that reverses the developmental potential of a cell or population of cells (e.g., a somatic cell). Stated another way, reprogramming refers to a process of driving a cell to a state with higher developmental potential, i.e., backwards to a less differentiated state. The cell to be reprogrammed can be either partially or terminally differentiated prior to reprogramming. In some embodiments of the aspects described herein, reprogramming encompasses a complete or partial reversion of the differentiation state, i.e., an increase in the developmental potential of a cell, to that of a cell having a pluripotent state. In some embodiments, reprogramming encompasses driving a somatic cell to a pluripotent state, such that the cell has the developmental potential of an embryonic stem cell, i.e., an embryonic stem cell phenotype. In some embodiments, reprogramming also encompasses a partial reversion of the differentiation state or a partial increase of the developmental potential of a cell, such as a somatic cell or a unipotent cell, to a multipotent state. Reprogramming also encompasses partial reversion of the differentiation state of a cell to a state that renders the cell more susceptible to complete reprogramming to a pluripotent state when subjected to additional manipulations, such as those described herein. Such manipulations can result in endogenous expression of particular genes by the cells, or by the progeny of the cells, the expression of which contributes to or maintains the reprogramming. In certain embodiments, reprogramming of a cell using the synthetic, modified RNAs and methods thereof described herein causes the cell to assume a multipotent state (e.g., is a multipotent cell). In some embodiments, reprogramming of a cell (e.g. a somatic cell) using the synthetic, modified RNAs and methods thereof described herein causes the cell to assume a pluripotent-like state or an embryonic stem cell phenotype. The resulting cells are referred to herein as “reprogrammed cells,” “somatic pluripotent cells,” and “RNA-induced somatic pluripotent cells.” The term “partially reprogrammed somatic cell” as referred to herein refers to a cell which has been reprogrammed from a cell with lower developmental potential by the methods as disclosed herein, such that the partially reprogrammed cell has not been completely reprogrammed to a pluripotent state but rather to a non-pluripotent, stable intermediate state. Such a partially reprogrammed cell can have a developmental potential lower that a pluripotent cell, but higher than a multipotent cell, as those terms are defined herein. A partially reprogrammed cell can, for example, differentiate into one or two of the three germ layers, but cannot differentiate into all three of the germ layers.

The term “reprogramming factor” as used herein refers to a developmental potential altering factor, as that term is defined herein, such as a protein, RNA, or small molecule, the expression of which contributes to the reprogramming of a cell, e.g. a somatic cell, to a less differentiated or undifferentiated state, e.g. to a cell of a pluripotent state or partially pluripotent state. A reprogramming factor can be, for example, transcription factors that can reprogram cells to a pluripotent state, such as SOX2, OCT3/4, KLF4, NANOG, LIN-28, c-MYC, and the like, including as any gene, protein, RNA or small molecule, that can substitute for one or more of these in a method of reprogramming cells in vitro. In some embodiments, exogenous expression of a reprogramming factor, using the synthetic modified RNAs and methods thereof described herein, induces endogenous expression of one or more reprogramming factors, such that exogenous expression of one or more reprogramming factors is no longer required for stable maintenance of the cell in the reprogrammed or partially reprogrammed state. “Reprogramming to a pluripotent state in vitro” is used herein to refer to in vitro reprogramming methods that do not require and/or do not include nuclear or cytoplasmic transfer or cell fusion, e.g., with oocytes, embryos, germ cells, or pluripotent cells. A reprogramming factor can also be termed a “de-differentiation factor,” which refers to a developmental potential altering factor, as that term is defined herein, such as a protein or RNA that induces a cell to de-differentiate to a less differentiated phenotype that is a de-differentiation factor increases the developmental potential of a cell.

The term “similarity” as used herein refers to the exact amino acid to amino acid comparison of two or more polypeptides at the appropriate place, where amino acids are identical or possess similar chemical and/or physical properties such as charge or hydrophobicity. A “percent similarity” then can be determined between the compared polypeptide sequences. Techniques for determining nucleic acid and amino acid sequence identity also are well known in the art and include determining the nucleotide sequence of the mRNA for that gene (usually via a cDNA intermediate) and determining the amino acid sequence encoded thereby, and comparing this to a second amino acid sequence. In general, “identity” refers to an exact nucleotide to nucleotide or amino acid to amino acid correspondence of two polynucleotides or polypeptide sequences, respectively.

The term “small molecule” as used herein refers to a chemical agent which can include, but is not limited to, a peptide, a peptidomimetic, an amino acid, an amino acid analog, a polynucleotide, a polynucleotide analog, an aptamer, a nucleotide, a nucleotide analog, an organic or inorganic compound (e.g., including heterorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

The term “somatic cell” as used herein refers to any cell other than a germ cell, a cell present in or obtained from a pre-implantation embryo, or a cell resulting from proliferation of such a cell in vitro. Stated another way, a somatic cell refers to any cell forming the body of an organism, as opposed to a germline cell. In mammals, germline cells (also known as “gametes”) are the spermatozoa and ova which fuse during fertilization to produce a cell called a zygote, from which the entire mammalian embryo develops. Every other cell type in the mammalian body—apart from the sperm and ova, the cells from which they are made (gametocytes) and undifferentiated, pluripotent, embryonic stem cells—is a somatic cell: internal organs, skin, bones, blood, and connective tissue are all made up of somatic cells. In some embodiments the somatic cell is a “non-embryonic somatic cell,” by which is meant a somatic cell that is not present in or obtained from an embryo and does not result from proliferation of such a cell in vitro. In some embodiments the somatic cell is an “adult somatic cell,” by which is meant a cell that is present in or obtained from an organism other than an embryo or a fetus or results from proliferation of such a cell in vitro. Unless otherwise indicated, the compositions and methods for reprogramming a somatic cell described herein can be performed both in vivo and in vitro (where in vivo is practiced when a somatic cell is present within a subject, and where in vitro is practiced using an isolated somatic cell maintained in culture).

Any cell type, but other than germ cells, of mammalian origin (e.g., humans, mice, monkey, swine, rat etc.) can be used as starting material for the production of induced pluripotent stem cells (iPSC) in the methods of the present disclosure. Examples include keratinizing epithelial cells (e.g., keratinized epidermal cells), mucosal epithelial cells (e.g., epithelial cells of the superficial layer of tongue), exocrine gland epithelial cells (e.g., mammary gland cells), hormone-secreting cells (e.g., adrenomedullary cells), cells for metabolism or storage (e.g., liver cells), intimal epithelial cells constituting interfaces (e.g., type I alveolar cells), intimal epithelial cells of the obturator canal (e.g., vascular endothelial cells), cells having cilia with transporting capability (e.g., airway epithelial cells), cells for extracellular matrix secretion (e.g., fibroblasts), constrictive cells (e.g., smooth muscle cells), cells of the blood and the immune system (e.g., T lymphocytes), sense-related cells (e.g., bacillary cells), autonomic nervous system neurons (e.g., cholinergic neurons), sustentacular cells of sensory organs and peripheral neurons (e.g., satellite cells), nerve cells and glia cells of the central nervous system (e.g., astroglia cells), pigment cells (e.g., retinal pigment epithelial cells), progenitor cells thereof (tissue progenitor cells) and the like. There is no limitation on the degree of cell differentiation, age of animal from which cells are collected and the like; even undifferentiated progenitor cells (including somatic stem cells) and finally differentiated mature cells can be used alike as sources of somatic cells in the methods of the present disclosure.

The choice of individual mammal as a source of somatic cells is not particularly limited; however, when the iPS cells obtained are to be used for regenerative medicine in humans, it is particularly advantageous, from the viewpoint of prevention of graft rejection, that somatic cells are patient's own cells or collected from another person (donor) having the same or substantially the same HLA type as that of the patient. The statement that the HLA type is “substantially the same” means that there is an agreement of the HLA types to the extent that allows a cell graft to survive in a patient receiving cells obtained by inducing differentiation from the somatic cell-derived iPS cell, transplanted with the use of an immunosuppressant and the like. Examples include cases where the primary HLA types (e.g., 3 loci HLA-A, HLA-B and HLA-DR) are the same and the like. When the iPS cells obtained are not to be administered (transplanted) to a human, but used as, for example, a source of cells for screening for evaluating a patient's drug susceptibility or adverse reactions, it is likewise desirable to collect the somatic cells from the patient or another person with the same genetic polymorphism correlating with the drug susceptibility or adverse reactions.

Somatic cells separated from a mammal such as mouse or human can be pre-cultured using a medium known per se suitable for the cultivation thereof, depending on the kind of the cells. Examples of such media include, but are not limited to, a minimal essential medium (MEM) comprising about 5 to 20% fetal calf serum, Dulbecco's modified Eagle medium (DMEM), RPMI1640 medium, 199 medium, F12 medium, and the like. When a transfer reagent such as cationic liposome, for example, is used in bringing the cell into contact with a nuclear reprogramming substance (and, as required, also with the iPS cell establishment efficiency improver described below), it is sometimes advantageous to exchange the medium with a serum-free medium in order to prevent transfer efficiency reductions.

The terms “Sox” and “Sox polypeptide” as used herein refers to any of the naturally-occurring members of the SRY-related HMG-box (Sox) transcription factors, characterized by the presence of the high-mobility group (HMG) domain, or variants thereof that maintain transcription factor activity, e.g., within at least 50%, 80%, or 90% activity compared to the closest related naturally occurring family member or polypeptides comprising at least the DNA-binding domain of the naturally occurring family member, and can further comprise a transcriptional activation domain. Sox polypeptides include, e.g., Sox1, Sox-2, Sox3, Sox4, Sox5, Sox6, Sox7, Sox8, Sox9, Sox10, Sox11, Sox12, Sox13, Sox14, Sox15, Sox17, Sox18, Sox-21, and Sox30. Sox1 has been shown to yield iPS cells with a similar efficiency as Sox2, and genes Sox3, Sox15, and Sox18 have also been shown to generate iPS cells, although with somewhat less efficiency than Sox2. See, Nakagawa et al., (2007) Nature Biotech. 26:101-106. In some embodiments, variants have at least 85%, 90%, or 95% amino acid sequence identity across their whole sequence compared to a naturally occurring Sox polypeptide family member such as those listed above or such as listed in Genbank accession number CAA83435 (human Sox2). Sox polypeptides (e.g., Sox1, Sox2, Sox3, Sox15, or Sox18) can be from human, mouse, rat, bovine, porcine, or other animals. Generally, the same species of protein will be used with the species of cells being manipulated.

The terms “stem cell” or “undifferentiated cell” as used herein refer to a cell in an undifferentiated or partially differentiated state that has the property of self-renewal and has the developmental potential to differentiate into multiple cell types, without a specific implied meaning regarding developmental potential (i.e., totipotent, pluripotent, multipotent, etc.). A stem cell is capable of proliferation and giving rise to more such stem cells while maintaining its developmental potential. In theory, self-renewal can occur by either of two major mechanisms. Stem cells can divide asymmetrically, which is known as obligatory asymmetrical differentiation, with one daughter cell retaining the developmental potential of the parent stem cell and the other daughter cell expressing some distinct other specific function, phenotype and/or developmental potential from the parent cell. The daughter cells themselves can be induced to proliferate and produce progeny that subsequently differentiate into one or more mature cell types, while also retaining one or more cells with parental developmental potential. A differentiated cell may derive from a multipotent cell, which itself is derived from a multipotent cell, and so on. While each of these multipotent cells may be considered stem cells, the range of cell types each such stem cell can give rise to, i.e., their developmental potential, can vary considerably. Alternatively, some of the stem cells in a population can divide symmetrically into two stem cells, known as stochastic differentiation, thus maintaining some stem cells in the population as a whole, while other cells in the population give rise to differentiated progeny only. Accordingly, the term “stem cell” refers to any subset of cells that have the developmental potential, under particular circumstances, to differentiate to a more specialized or differentiated phenotype, and which retain the capacity, under certain circumstances, to proliferate without substantially differentiating. In some embodiments, the term stem cell refers generally to a naturally occurring parent cell whose descendants (progeny cells) specialize, often in different directions, by differentiation, e.g., by acquiring completely individual characters, as occurs in progressive diversification of embryonic cells and tissues. Some differentiated cells also have the capacity to give rise to cells of greater developmental potential. Such capacity may be natural or may be induced artificially upon treatment with various factors. Cells that begin as stem cells might proceed toward a differentiated phenotype, but then can be induced to “reverse” and re-express the stem cell phenotype, a term often referred to as “dedifferentiation” or “reprogramming” or “retrodifferentiation” by persons of ordinary skill in the art.

The term “substantially pure”, when used in reference to stem cells or cells derived therefrom (e.g., differentiated cells), means that the specified cells constitute the majority of cells in the preparation (i.e., more than 20%, 30%, 40%, 50%, 60%, 70%, 80%, 90%, or 95%). Generally, a substantially purified population of cells constitutes at least about 70% of the cells in a preparation, usually about 80% of the cells in a preparation, and particularly at least about 90% of the cells in a preparation (e.g., 95%, 97%, 99% or 100%). As such, a method of the disclosure provides the advantage that a substantially pure population of a particular type of cells can be obtained without contamination by other cell types.

The term “terminal differentiation” refers to the final differentiation of a cell into a mature, fully differentiated cell. For example, neural progenitor cells and muscle progenitor cells can differentiate into hematopoietic cell lineages, terminal differentiation of which leads to mature blood cells of a specific cell type. Usually, terminal differentiation is associated with withdrawal from the cell cycle and cessation of proliferation.

The term “totipotency” refers to a cell with a developmental potential to make all of the cells in the adult body as well as the extra-embryonic tissues, including the placenta. The fertilized egg (zygote) is totipotent, as are the cells (blastomeres) of the morula (up to the 16-cell stage following fertilization).

The term “transcription factor” as used herein refers to a protein that binds to specific parts of DNA using DNA binding domains and is part of the system that controls the transcription of genetic information from DNA to RNA.

### Description

This disclosure relates to use of BRD3R to increase the efficiency of the induction of a cell population, prepared from non-embryonic origins to pluripotent stem cells. This population can be obtained easily at a very high yield. BRD3R can be used, therefore, to regenerate differentiated, functional cells useful in treating various degenerative disorders or tissue damage. As shown in the examples below, the population can be easily prepared, and then maintained, and expanded in vitro, and induced to differentiation using routine technical approaches. Containing a normal chromosomal complement, these stem cells are lineage-uncommitted and can form all somatic (non-reproductive) cells of the body. They can also form the reproductive gametes sperm and/or ovum, and cells and tissues of the embryonic and fetal portions of the placenta. These stem cells are responsive to lineage-induction agents, proliferation agents, and differentiation inhibitory agents. Due to these advantages, they represent an alternative to other stem cells.
Identification of BRD3R as a robust human reprogramming factor: It was hypothesized that there are undiscovered reprogramming factor(s) to account for the higher efficiency and faster kinetics of SCNT compared to factor reprogramming. To search for such reprogramming factor(s), a human kinase cDNA expression library was prepared and screened on account of the importance of phosphorylation in general cell biology and in pluripotency in particular. The importance of phosphorylation in pluripotency and reprogramming is suggested by there being are 8,359 phosphorylation sites in human embryonic stem cells (hESC) (Swaney et al., (2008) Nat. Methods 5: 959-964), the majority of which are believed to be differentially phosphorylated relative to somatic cells (Phanstiel et al., (2011) Nat. Methods 8: 821-827).

Accordingly, a sensitive protocol was first established that enabled simultaneous screening of 22 individual cDNAs in a long process as reprogramming (FIGS. 1A, and 6A-6K). The reprogramming protocol of the disclosure includes 3 of the Yamanaka factors OCT4, SOX2 and KLF4 (3F). MYC was excluded because, consistent with previous report, MYC was slightly detrimental to reprogramming in the feeder-free/serum-free E8 system (FIGS. 1D and 1E) (Chen et al., (2011) Nat. Methods 8: 424-429; Banito et al., (2009) Genes Dev. 23: 2134-2139).

Based on a primary screen of 89 human kinase cDNAs (FIG. 1B and Table 1), 11 candidate cDNAs were re-screened (FIG. 1C and Table 2).

TABLE 1
The 89 kinase cDNAs screened

| Clone ID | Addgene | gene | FC | SD | n |
|---|---|---|---|---|---|
| L1 | P1A1 | CAMK2B | 1.145 | | 1 |
| L10 | P1A10 | CSNK2B | 1.747 | | 1 |
| L11 | P1A11 | PRKAB1 | 1.072 | | 1 |
| L12 | P1A12 | LOC389599 | 0.944 | | 1 |
| L13 | P1B1 | CAMKK1 | 2.603 | 1.105 | 3 |
| L14 | P1B2 | DYRK4 | 0.934 | | 1 |
| L15 | P1B3 | CIB1 | 1.786 | | 1 |
| L16 | P1B4 | GK2 | 0.993 | | 1 |
| L17 | P1B5 | PRKCI | 1.395 | | 1 |
| L18 | P1B6 | FLJ25006 | 0.872 | | 1 |
| L19 | P1B7 | CDK3 | 1.120 | 0.183 | 3 |
| L2 | P1A2 | NEK3 | 1.474 | | 1 |
| L20 | P1B8 | PION | 2.108 | 0.207 | 3 |
| L21 | P1B9 | STK19 | 0.950 | 0.192 | 3 |
| L22 | P1B10 | GRK6 | 1.099 | | 1 |
| L23 | P1B11 | LOC442075 | 1.086 | | 1 |
| L24 | P1B12 | LOC390877 | 0.770 | | 1 |
| L25 | P1C1 | AURKC | 0.638 | | 1 |
| L26 | P1C2 | FLJ23356 | 0.977 | | 1 |
| L27 | P1C3 | PRKAR1B | 0.997 | | 1 |
| L28 | P1C4 | FLJ40852 | 0.990 | | 1 |
| L29 | P1C5 | SGK2 | 1.509 | 0.681 | 3 |
| L3 | P1A3 | ACVR1 | 1.178 | | 1 |
| L30 | P1C6 | TESK1 | 0.760 | | 1 |
| L31 | P1C7 | GUK1 | 1.611 | 0.621 | 3 |
| L32 | P1C8 | DCK | 1.130 | | 1 |
| L33 | P1C9 | CDKL4 | 3.870 | | 1 |
| L34 | P1C10 | PRKX | 2.348 | | 1 |
| L35 | P1C11 | PANK3 | 2.239 | | 1 |
| L36 | P1C12 | PHKG1 | 1.522 | | 1 |
| L37 | P1D1 | CIB4 | 2.543 | | 1 |
| L38 | P1D2 | PKDCC | 5.065 | | 1 |
| L39 | P1D3 | NUAK2 | 2.674 | | 1 |
| L4 | P1A4 | NME1-NME2 | 1.016 | | 1 |
| L40 | P1D4 | CDKL1 | 1.735 | 0.526 | 3 |
| L41 | P1D5 | DAPK2 | 1.674 | | 1 |
| L411 | P5E9 | PBK | 1.254 | 0.624 | 3 |
| L42 | P1D6 | LOC649228 | 1.435 | | 1 |
| L43 | P1D7 | NME2 | 2.175 | 1.116 | 3 |
| L436 | P5E4 | CDK2 | 1.457 | | 1 |
| L44 | P1D8 | ITGB1BP3 | 3.652 | | 1 |
| L45 | P1D9 | LOC652779 | 4.913 | | 1 |
| L46 | P1D10 | NEK7 | 2.826 | | 1 |
| L47 | P1D11 | SLAMF6 | 2.674 | | 1 |
| L48 | P1D12 | TSSK2 | 3.000 | | 1 |
| L49 | P1E1 | RAF1 | 1.326 | | 1 |
| L5 | P1A5 | CRKL | 1.352 | | 1 |
| L50 | P1E2 | BMP2KL | 4.087 | | 1 |
| L51 | P1E3 | CSNK1G3 | 1.174 | | 1 |
| L52 | P1E4 | ACVR2B | 1.217 | | 1 |
| L53 | P1E5 | GRK7 | 2.891 | | 1 |
| L54 | P1E6 | PAK6 | 1.790 | 1.503 | 3 |
| L55 | P1E7 | MARK2 | 0.978 | | 1 |
| L56 | P1E8 | SNRK | 2.318 | 0.964 | 2 |
| L560 | | SGK494 | 1.194 | 0.332 | 3 |
| L57 | P1E9 | MAP2K1 | 3.023 | 0.675 | 2 |
| L58 | P1E10 | PLXNB2 | 2.370 | 0.170 | 2 |
| L59 | P1E11 | CAMK2A | 2.643 | 2.272 | 2 |
| L6 | P1A6 | CSNK1A1 | 1.128 | | 1 |
| L60 | P1E12 | CAMK2G | 1.883 | 0.809 | 3 |
| L61 | P1F1 | BRD3 | 7.989 | 6.026 | 2 |
| L62 | P1F2 | MPP7 | 3.441 | 1.498 | 2 |
| L63 | P1F3 | IRAK2 | 1.845 | 0.959 | 3 |
| L64 | P1F4 | PRKG1 | 2.670 | 0.820 | 2 |
| L65 | P1F5 | PRKFB1 | 2.848 | 2.690 | 2 |
| L66 | P1F6 | IPPK | 2.430 | 1.867 | 2 |
| L67 | P1F7 | MPP6 | 1.525 | 1.025 | 2 |
| L68 | P1F8 | MPP4 | 2.282 | 1.016 | 2 |
| L69 | P1F9 | NEK8 | 3.182 | 2.571 | 2 |
| L7 | P1A7 | CCL4 | 1.528 | 0.192 | 3 |
| L70 | P1F10 | PANK4 | 2.052 | 1.694 | 2 |
| L71 | P1F11 | MAPK8 | 1.486 | 0.829 | 3 |
| L72 | P1F12 | CDKL2 | 1.734 | 0.730 | 2 |
| L73 | P1G1 | CAMKK2 | 1.652 | 0.569 | 2 |
| L74 | P1G2 | BMP2K | 1.874 | 0.891 | 3 |
| L75 | P1G3 | PRKCQ | 4.573 | 3.433 | 2 |
| L76 | P1G4 | LIMK2 | 3.491 | 3.548 | 2 |
| L77 | P1G5 | MAPKAPK2 | 1.338 | 0.561 | 3 |
| L78 | P1G6 | PGK1 | 2.532 | 2.783 | 2 |
| L79 | P1G7 | CHKA | 1.365 | 0.864 | 3 |
| L8 | P1A8 | STK33 | 1.477 | | 1 |
| L80 | P1G8 | CDC2L6 | 2.273 | 2.443 | 2 |
| L81 | P1G9 | ADRBK1 | 1.145 | 0.350 | 3 |
| L82 | P1G10 | MPP3 | 2.984 | 2.497 | 2 |
| L83 | P1G11 | BRSK2 | 4.559 | 2.745 | 2 |
| L84 | P1G12 | UHMK1 | 5.750 | | 1 |
| L9 | P1A9 | C1orf57 | 1.089 | | 1 |
| L93 | P1H9 | RIPK1 | 0.990 | 0.453 | 3 |
| L94 | P1H10 | KSR | 1.394 | 0.734 | 3 |

FC, fold change; SD, standard deviation; n, number of repeats; Addgene, listed in this A BRD3 cDNA (library identifier, L61) exhibited a 27.6-fold increase in reprogramming activity as judged by the number of ALP⁺ colonies (Clone 61 in FIG. 1C and Table 2). The L61 cDNA plasmid was purified and verified the robust reprogramming activity based on the number of TRA-1-60⁺ colonies (FIGS. 1D, 1E, and 7). cDNA L61, identified as an isoform of human BRD3 (GenBank Accession Number, BC032124; protein GenBank Accession Number, AAH32124; 556 aa (SEQ ID NO: 47)). AAH32124 differs from the canonical BRD3 (mRNA, NM_007371, protein, NP_031397; 726 aa (SEQ ID NO: 45)) in the carboxyl-terminus.

In place of the ET domain, this protein BRD3R (BRD3 with Reprogramming activity) has a unique extension of eight amino acids. The reprogramming-enhancing BRD3R is expressed in human cells as an atypical isoform by alternative splicing of BRD3 gene with its expression elevated in PSCs compared to somatic cells. Other members of the bromodomain BET proteins examined have no enhanced reprogramming activity.

High-Quality Primary iPSC Colonies by BRD3R Reprogramming

When combined with the 3F combination (OCT4, SOX2 and KLF4), BRD3R gave rise to abundant TRA-1-60⁺ clusters as early as day 6, whereas such clusters were infrequent events before day 10 in the control reprogramming (3F alone and 3F-GFP) (FIGS. 8A and 8E). On day 10, TRA-1-60⁺ cells in BRD3R dishes developed into colonies while controls contained only small clusters of TRA-1-60⁺ cells (FIG. 8E). The number of high-quality PSC colonies was also compared (well-defined colony border, homogeneous iPSCs within each reprogramming colony, smooth colony surface, and typical morphology of pluripotent cells (small cells, large nucleus, and close cell contact)), BRD3R reprogramming gave rise to at least 57× more colonies with PSC morphology than controls (FIGS. 9A and 9B). In addition, the iPSC colonies generally were larger in BRD3R dishes than in the control dishes (FIG. 1E, and FIGS. 8C, 8E, 9B, and 12C). Also, BRD3R reprogramming generated more TRA-1-60⁺ colonies, a more reliable marker (FIGS. 1D, 1E, 8B-8D, 12B, and 12C). These observations suggest a higher quality of reprogramming by BRD3R overexpression.

The iPSCs generated using BRD3R (designated as 3RiPSC) are pluripotent as demonstrated by several criteria. They expressed pluripotent markers (OCT4, SOX2, NANOG, LIN28, TRA-1-81, TRA-1-60, SSEA3 and SSEA4) (FIG. 10A), produced teratomas with cells representing all the three embryonic germ layers (FIG. 10B), generated embryoid bodies (FIG. 10C), differentiated into multiple lineages in vitro (FIG. 10D), silenced transgenes (FIG. 10E), and acquired a transcriptome highly similar to those of hESCs (FIG. 11).

The 3RiPSCs also demonstrated a typical pluripotent cell cycle structure with a truncated G1 phase and an increased cell population in S/G2/M phases (FIG. 12F) and had normal karyotypes (FIG. 10G). Thus, BRD3R robustly increases reprogramming efficiency, speeds up reprogramming kinetics, and enhances the quality of reprogramming.

### BRD3R Uniquely Possesses Reprogramming Activity

BRD3R belongs to the BET subfamily of bromodomain proteins that includes four members, BRD2, BRD3, BRD4 and BRDT (FIG. 12A). They are characterized by two bromodomains and an extra terminal domain (ET domain). BRD2, BRD4 and the canonical BRD3 were examined as to whether they also exhibit reprogramming activity. Surprisingly, none did (FIGS. 12B and 12C).

Inhibition with BET-specific inhibitors significantly impaired reprogramming (FIGS. 13A and 13B). However, these inhibitors cannot distinguish among BET members. Therefore, an shRNA was designed that specifically targets BRD3R (FIG. 13C). Inhibition of BRD3R impaired reprogramming by 58% (FIG. 13D), suggesting a role for BRD3R in reprogramming in addition to the reprogramming promoting activity of this protein.

RT-PCR with isoform-specific primers demonstrated that BRD3R is expressed in human BJ cells, and the expression was elevated in hESCs compared to BJ cells (FIG. 14B). RT-qPCR with isoform-specific primers for both isoforms gave similar results (FIG. 14C). This was further verified using a BRD3 antibody that recognizes the common region of the two isoforms (FIG. 14D). The multiple RNA sequencing data corroborated the higher expression of BRD3/BRD3R in PSC compared to somatic cells. Interestingly, BRD3R had a much lower expression than BRD3 both in somatic cells and PSCs.

To test whether the unique 8-aa tail of BRD3R is responsible to the observed reprogramming activity, this 8-aa tail was deleted. No decrease in reprogramming activity for this deletion mutation was observed. Since there is a deletion of 178 aa at the C terminus in BRD3R (SEQ ID NO: 47) compared with the sequence of BRD3 (SEQ ID NO: 45) and the nuclear localization signal is not defined, its ability to localization into nucleus was examined.

BRD3R was localized into nucleus when overexpressed in BJ cells (FIG. 14F). One basic biochemical feature of BET proteins is binding to acetylated histone in regions of euchromatins BRD3R and HP1α, a marker of heterochromatin, were co-stained. These two proteins were localized to distinct chromatin regions (lower row, FIG. 14G). In contrast, BRD3R co-localized extensively with H3-K9Ac (FIG. 14G), a marker of euchromatin. Study with confocal imaging demonstrated that BRD3R associates with mitotic chromatin.

To substantiate BRD3R binding to the acetylated chromatins, in vitro peptide-pull-down experiments were performed using 8 peptides with various histone acetylation modifications (as shown in Table 4). Cellular proteins from human fibroblasts that overexpressed BRD3R or BRD3 were used considering that other cellular factors may be beneficial or essential for binding.

BRD3R bound strongly to tetra-acetylated H4 (H4K5/8/12/16Ac), and weakly to H4K5Ac (upper right, FIG. 14H). BRD3R also bound to H3K9Ac and H3K14Ac, but bound very weakly to biacetylated H3 (H3K9/14Ac) (upper left, FIG. 14H). Binding of BRD3 to H4K5/8/12/16 only was detected, and the binding was weaker than BRD3R based on the relative amount of pull-down to input (FIG. 14H). Thus, BRD3R uniquely possesses reprogramming activity. This unusual isoform is expressed in both human somatic cells and PSCs. BRD3R localizes into nucleus in regions distinct from those bound by HP1α, but overlapping with H3K9Ac foci and the two BRD3R isoforms demonstrated differential binding to the acetylated histones.
BRD3R facilitates resetting of the pluripotent cell cycle structure and increases the number of mitotic cells in the early stages of reprogramming: BRD3R may promote reprogramming by downregulating reprogramming barriers. The p53-p21 pathway is a well-recognized barrier to reprogramming. Manipulation of p53-p21 members promotes reprogramming by increasing proliferation rate of the reprogramming cells in the early stages (Guo et al., (2014) Cell 156: 649-662; Banito et al., (2009) Genes Dev. 23: 2134-2139). In contrast, comparable overall proliferation rates between BRD3R and control cells before day 9 were seen (FIG. 15C). Between days 9 and 11, there was an abrupt increase in cell numbers in BRD3R reprogramming compared to controls, agreeing with the iPSC colonies appearing earlier in BRD3R reprogramming. Therefore, the rapid proliferation of the already reprogrammed cells inside these expanding BRD3R colonies contributed to the increased numbers of cells at this stage.

Activation of the p53-p21 pathway during reprogramming increases cell apoptosis and senescence (Banito et al., (2009) Genes Dev. 23: 2134-2139; Li et al., (2009) Nature 460: 1136-1139); however, there were similar levels of apoptosis in BRD3R reprogramming compared to control reprogramming (FIG. 15D). Reduced cell senescence, however, were seen during the early stages in BRD3R reprogramming based on SA-β-galactosidase staining (FIGS. 2D and 2E). The decreased cell senescence does not result from the compromised p53-p21 pathway, but may result, at least in part, from the ability of BRD3R to promote mitosis. RNA sequencing with early reprogramming cells were then performed.

Downregulation was not seen for CDKN2A (p16^{ink4a}/p19^{Arf}), CDKN2B (p15^{ink4b}), CDKN1A (p21^{CIP1}) or TP53 (p53) in BRD3R reprogramming cells based on the multiple RNA sequencing data. Nor was differential expression seen for the regulator genes of the p53-p21 pathway, MDM2 and MDM4. In contrast, upregulation of the CDK inhibitor gene p57^{Kip2} (CDKN1C) by BRD3R in the early stages of reprogramming (FIG. 3C) was consistently seen. Interestingly, unlike cell cycle inhibitors of the p53-p21 pathway, CDKN1C is annotated as a mitotic gene. Another CDK inhibitor gene CDKN2C (p18^{ink4c}) is also among 185 mitotic genes upregulated by BRD3R.

There were significant changes in cell morphology in early stages in BRD3R reprogramming in that it gave rise to more small compact cells (FIG. 2E) reminiscent of mitotic cells. Flow cytometry demonstrated that BRD3R statistically increased the population of cells in G2/M phases, and statistically reduced the number of cells in G1 on day 6 of reprogramming compared to controls (FIGS. 2A and 2B). Mitotic shake-off experiments were further performed with day-4 reprogramming cells, and significantly more mitotic cells (2.43×) were collected from the BRD3R reprogramming dishes compared to controls (FIG. 2C). These data indicate that BRD3R increases the number of mitotic cells in the early stages of reprogramming.

To provide insights into possible mechanisms involved in BRD3R induction of mitotic cells during reprogramming, confocal immunocytochemical localization of an HA-tagged BRD3R during the reprogramming process was performed. BRD3R remained associated with mitotic chromatin at all stages of mitosis (FIG. 2F). In contrast, Pol II dissociated from mitotic chromatin as expected. Collectively, BRD3R promotes reprogramming not by enhancing proliferation of the reprogramming cells and regulation of the p53-p21 pathway, but by increasing the number of reprogramming-privileged mitotic cells via its continuous association with mitotic chromatin in the early stages of reprogramming.
BRD3R upregulates a large set of human mitotic genes at an early stage of reprogramming: To understand further the reprogramming mechanism of BRD3R, the transcriptional contribution of BRD3R overexpression to reprogramming was investigated by performing RNA sequencing analysis of cells on day 3 of reprogramming. While not wishing to be bound by any one theory, it is contemplated that these cells are still homogeneous at this very early stage and that BRD3R-overexpression may have an early molecular impact on reprogramming as it speeds up reprogramming by several days.

The fold changes resulting from BRD3R overexpression were calculated using averaged DEseq normalized read counts (ADNRC) (average read counts (ARC) and average fold changes (AFC)). RNA sequencing identified 401 genes (≥1.7×, p<0.05, ADNRC≥50 for BRD3R treatments) upregulated in BRD3R-expressing cells compared to controls. To identify the biological significance, a GO analysis (biological process) was performed. Of the 401 genes, 335 were mapped with GO terms in the PANTHER GO database. A total of 128 BRD3R-upregulated genes belong to the mitotic category, representing 38.2% of 335 GO mapped genes, and 31.9% of 401 BRD3R-upregulated genes (FIG. 3C).

Fold changes were also calculated using individual DEseq normalized read counts (IDNRC) for two independent sets of RNA sequencing data (individual read counts (IRC) and individual fold changes (IFC)). There were 57 additional mitotic genes (a total of 185 mitotic genes) statistically upregulated (≥1.5×, p<0.05) by BRD3R overexpression on day 3 of reprogramming in at least one differential expression comparison if both individual and average fold increases were considered (5 comparisons, 4 IFC and 1 AFC). The AFC was, therefore, re-examined for these 185 mitotic genes without consideration of their p values. Three genes did not show upregulation by BRD3R (CEP78, PSMB9 and ERG, 0.94×, 0.87×, and 0.84×, respectively). The remaining 182 mitotic genes demonstrated at least a 1.2-fold increase, and 168 of these mitotic genes displayed at least a 1.5-fold increase. Most stringently, 23 of these mitotic genes were always upregulated (sorting criteria, FC≥1.5×, p<0.05) in all of the differential expression analyses (FIGS. 3C, 3E, and 22A-22F), and these genes have an AFC of at least 2.24× (p<0.05) (FIG. 3E). 11 mitotic genes were randomly selected from the 185 BRD3R-upregulated genes and performed RT-qPCR verification. These 11 genes were all upregulated by BRD3R on day 3 of reprogramming (FIG. 3D). Thus, BRD3R up-regulates a set of mitotic genes in early stages of reprogramming.
The BRD3R-upregulated mitotic genes constitute an expression fingerprint of PSC: The relative expression levels of the 23 mitotic genes were examined in PSC compared to somatic cells. RNA sequencing of two hESCs (H1 and H9), two human iPSC lines (3RiPSC3 and 3RiPSC4), BJ cells (3 replicates) and one isolate of human keratinocytes were performed. KIF20A was also included in the analysis because it is also consistently upregulated by BRD3R, but the p value was marginal (p=0.057) in the AFC comparison.

The results showed that 19 of the 24 BRD3R-regulated mitotic genes are consistently upregulated in human PSCs (both ESC and iPSC) (FIGS. 4B, 4C, 19, 20A, and 20B). The dataset GSE34200 from the NIH human PSC expression database includes microarray expression data for the 21 human ESC lines registered at NIH (132 microarray samples), 8 human iPSC lines (46 microarray samples) and 20 human somatic tissues (Mallon et al., (2013) Stem Cell Res. 10: 57-66). The analysis of this dataset showed that all the 24 BRD3R-upregulated mitotic genes exhibited higher expression in PSC, whereas the two control somatic genes (LMNA and CDKN1A) demonstrated a higher expression in somatic cells (FIG. 4A). Housekeeping genes (ACTB and GUSB) had similar expression levels between PSCs and somatic tissues, and the established pluripotent genes (NANOG and POU5F1) have higher expression in PSCs.

RT-qPCR was also performed to compare the expression levels of the 11 mitotic genes that were verified previously in reprogramming cells before. These 11 mitotic genes all exhibited elevated expression in human PSCs (FIGS. 4D and 4E). Therefore, at least 19 of the BRD3R-upregulated mitotic genes are upregulated in PSCs, and therefore these 19 mitotic genes constitute a novel molecular fingerprint of the PSC transcriptome.

A human kinase library was screened to identify BRD3R as a robust reprogramming factor. Among the 24 mitotic genes consistently upregulated by BRD3R in the early stages of reprogramming, four have kinase activities (AURKB, CCNB1, CDK1 and PBK); five regulate kinase activities (CCNA1, CDC6, CDKN1C, CKS2, KIF20A), and one is a phosphatase (DLGAP5) (Table 2). CDK1 is a master mitotic kinase, and AURKB is a critical mitotic kinase. Therefore, even if BRD3R may not have kinase activity, this protein likely regulates an important mitotic kinase network to promote reprogramming.

TABLE 2
Summary of secondary screen of candidate human kinase cDNAs for
reprogramming activity

| | | | FC to GFP | | | |
|---|---|---|---|---|---|---|
| treatment | | Gene | Experiment | Experiment | Average | |
| ID | Addgene | symbol | 1 | 2 | FC | SD |
| 3F | N/A | N/A | 1.8079 | 1.8187 | 1.8133 | 0.0076 |
| GFP + 3F | NA | N/A | 1 | 1 | 1 | 0 |
| L10 + 3F | P1A10 | CSNK2B | 2.9470 | 3.3859 | 3.1665 | 0.3104 |
| L13 + 3F | P1B1 | CAMKK1 | 2.5166 | 2.5556 | 2.5361 | 0.0276 |
| L15 + 3F | P1B3 | CIB1 | 2.9735 | 3.3158 | 3.1447 | 0.2420 |
| L20 + 3F | P1B8 | PION | 2.7616 | 2.7602 | 2.7609 | 0.0009 |
| L33 + 3F | P1C9 | CDKL4 | 2.9337 | 2.5731 | 2.7534 | 0.2550 |
| L38 + 3F | P1D2 | PKDCC | 1.6026 | 1.2105 | 1.4066 | 0.2772 |
| L44 + 3F | P1D8 | ITGB1BP3 | 3.0132 | 0.7661 | 1.8897 | 1.5889 |
| L45 + 3F | P1D9 | LOC652779 | 3.1854 | 2.0701 | 2.6278 | 0.7886 |
| L50 + 3F | P1E2 | BMP2KL | 2.2053 | 1.2807 | 1.7429 | 0.6538 |
| L61 + 3F | P1F1 | BRD3 | 33.8823 | 21.32 | 27.6012 | 8.8829 |
| L84 + 3F | P1G12 | UHMK1 | 1.8873 | 1.6797 | 1.7835 | 0.1468 |

The reprogramming with each candidate gene was repeated once in the secondary screen. FC, fold change compared with OSK-GFP control reprogramming. Reprogramming activity was evaluated by numbers of ALP⁺ colonies on day 25 of reprogramming with E8 system. Addgene, Addgene plate location number; SD, standard deviation; 3F, 3 factors: OCT4, SOX2 and KLF4.

BRD3R exhibited robust reprogramming activity whereas other BET members including the canonical BRD3 did not. BET family members demonstrate similarity in primary sequence, 3D structure, biochemical features and cellular activities. The major common biochemical property for BET proteins is their ability to bind to acetylated lysine on histone tail. Unlike transcription factors, BET proteins remain associated with mitotic chromatin. Except for BRDT, BET members are ubiquitously expressed. BRD2 and BRD3 both regulate active genes, but they differentially bind to some active genes (LeRoy et al., (2008) Mol. Cell 30: 51-60). Knockdown of BRD3 in HEK293 cells leads to cell death, but knockdown of BRD2 does not (LeRoy et al., (2008) Mol. Cell 30: 51-60). BRD4 also has two isoforms, but the two isoforms localize to different cellular compartments, interact with different proteins, display different binding profile for acetylated histone, and have distinct biological roles (Alsarraj et al., (2013) PLoS One 8: e80746). The data establish that BRD3R uniquely possesses the reprogramming activity.

The ARF-p53 pathway can prevent reprogramming of cells with DNA damage (Marion et al., (2009) Nature 460: 1149-1153), but it also constitutes a reprogramming barrier (Banito et al., (2009) Genes Dev. 23: 2134-2139; Li et al., (2009) Nature 460: 1136-1139). Many reprogramming protocols employ shRNA knockdown of the p53-p21 pathway to enhance reprogramming. However this manipulation increases the risk of introduction of reprogramming-associated mutations into iPSCs. The data demonstrates that BRD3R does not impair the ARF-p53 surveillance pathway, thus ensuring the integrity of reprogrammed genomes.

Although there are 24 mitotic genes (FIG. 16B) among the 106 genes consistently upregulated by BRD3R at early stage of reprogramming, 36 of the remaining 82 non-mitotic genes are annotated with the GO term, “developmental process”. It is not clear whether these non-mitotic genes contribute to the observed reprogramming activity of BRD3R. Only 45 genes are consistently down-regulated by BRD3R. Among the 45 genes, 17 genes turn out to be upregulated in human fibroblasts compared to human PSCs. Although these 17 genes are not consistently upregulated in human somatic tissues (FIG. 18), the downregulations in reprogramming fibroblasts may contribute to some of the observed reprogramming activity of BRD3R

The results allow the establishment of a model on how BRD3R modulates reprogramming as shown in FIG. 5. In the early stages of reprogramming, BRD3R upregulates a large set of mitotic genes via direction association with mitotic chromatin, which increases the population of mitotic cells. These mitotic cells are privileged cells for reprogramming. Positive regulation of the 19 PSC fingerprint mitotic genes by BRD3R may also contribute to the transcriptional resetting of these genes to their elevated levels of expression in PSCs. The model is in agreement with previous observations that only mitotic cells (M-II oocytes and mitotic zygotes) have sufficient reprogramming power to enable cloning of animals (Wakayama et al., (2000) Nat. Genet. 24: 108-109; Egli et al., (2007) Nature 447: 679-685), and that donor nuclei also have mitotic advantage in reprogramming (Halley-Stott et al., (2014) PLoS Biol. 12: e1001914), indicating a paramount importance of mitosis in reprogramming. PSCs have a unique cell cycle structure characterized with a shortened G1 phase (White & Dalton (2005) Stem Cell Rev. 1: 131-138). During reprogramming process, the somatic cell cycle structure must be reset to that of PSCs. However, the mechanisms for this resetting are poorly understood.

Overexpression of BRD3R decreased the number of cells in G1 and increased the number of cells in G2/M (FIGS. 2A and 2B). Thus, with these observations in combination of data supporting its role in regulation of mitosis during reprogramming, BRD3R may facilitate reprogramming by resetting the somatic cell cycle structure to that of PSCs as a result of its regulation of the 128 mitotic genes, and/or through regulation of other cell cycle genes.
Transferring a Nuclear Reprogramming factor into Somatic Cell: Transfer of a “nuclear reprogramming factor” e.g., BRD3R of the disclosure and a “nuclear reprogramming factor capable of inducing iPS cells when combined with BRD3R” into a somatic cell can be performed using a method of protein transfer into cells known in the art. Such methods include, for example, the method using a protein transfer reagent, the method using a protein transfer domain (PTD)- or cell-penetrating peptide (CPP)-fusion protein, the microinjection method and the like. Protein transfer reagents are commercially available, including those based on a cationic lipid, such as BioPOTER® Protein Delivery Reagent (Genlantis), Pro-Ject® Protein Transfection Reagent (PIERCE), PULSin® delivery reagent (Polyplus-transfection) and ProVectin (IMGENEX); those based on a lipid, such as Profect-1 (Targeting Systems); those based on a membrane-permeable peptide, such as Penetrain Peptide (Q biogene), Chariot Kit (Active Motif), and GenomONE (Ishihara Sangyo), which employs HVJ envelop (inactivated Sendai virus), and the like. The transfer can be achieved according to the protocols attached to these reagents. Nuclear reprogramming factor(s) can be diluted in an appropriate solvent (e.g., a buffer solution such as PBS or HEPES), a transfer reagent is added, the mixture is incubated at room temperature for about 5 to 15 mins to form a complex. This complex is then added to cells after exchanging the medium with a serum-free medium, and the cells are incubated at 37° C. for one to several hours. Thereafter, the medium is removed and replaced with a serum-containing medium.

A fusion protein expression vector incorporating a cDNA encoding a iPS cell establishment efficiency improver such as BRD3R according to the disclosure and a PTD or CPP sequence can be prepared to allow the recombinant expression of the fusion protein, and the fusion protein can be recovered for use in for transfer. This transfer can be achieved as described above, except that no protein transfer reagent is added.

Microinjection, a method of placing a protein solution in a glass needle having a tip diameter of about 1 μm, and injecting the solution into a cell, can ensure the transfer of the protein into the cell. Other useful methods of protein transfer include electroporation, the semi-intact cell method (Kano et al., (2006) Methods Mol. Biol. 322: 357-365), transfer using the Wr-t peptide (Kondo et al., (2004) Mol. Cancer. Ther. 3: 1623-1630) and the like.

The protein transfer operation can be performed one or more optionally chosen times (e.g., once or more to 10 times or less, or once or more to 5 times or less, and the like); advantageously, the transfer operation can be performed twice or more (e.g., 3 times or 4 times) repeatedly. The time interval for repeated transfer is, for example, 6 to 48 h, advantageously 12 to 24 h.

When emphasis is placed on iPS cell establishment efficiency, it can be advantageous to use the BRD3R of the disclosure in the form of a nucleic acid that encodes the same, rather than as the proteinaceous factor itself. The nucleic acid may be a DNA, an RNA, or a DNA/RNA chimera, and may be double-stranded or single-stranded. Most advantageously, the nucleic acid can be a double-stranded DNA, particularly cDNA.

A cDNA encoding the nuclear reprogramming factor, such as BRD3R, of the disclosure can be inserted into an appropriate expression vector comprising a promoter capable of functioning in a host somatic cell. Useful expression vectors include, but are not limited to, viral vectors such as retrovirus, lentivirus, adenovirus, adeno-associated virus, herpesvirus and Sendai virus, plasmids for the expression in animal cells (e.g., pA1-11, pXT1, pRc/CMV, pRc/RSV, and pcDNAI/Neo) and the like. The kind of vector used can be chosen as appropriate according to the intended use of the iPS cells obtained. Useful vectors include, for example, adenovirus vectors, plasmid vectors, adeno-associated virus vectors, retrovirus vectors, lentivirus vectors, Sendai virus vectors and the like.

Examples of promoters used in expression vectors include the EF1α promoter, the CAG promoter, the SRα promoter, the SV40 promoter, the LTR promoter, the CMV (cytomegalovirus) promoter, the RSV (Rous sarcoma virus) promoter, the MoMuLV (Moloney mouse leukemia virus) LTR, the HSV-TK (herpes simplex virus thymidine kinase) promoter and the like, with preference given to the EF1α promoter, the CAG promoter, the MoMuLV LTR, the CMV promoter, the SRα promoter and the like.

The expression vector may contain as desired, in addition to a promoter, an enhancer, a polyadenylation signal, a selectable marker gene, a SV40 replication origin and the like. Examples of useful selectable marker genes include the dihydrofolate reductase gene, the neomycin resistant gene, the puromycin resistant gene and the like.

The nucleic acids as nuclear reprogramming factors (reprogramming genes) may be separately integrated into different expression vectors, or 2 kinds or more, advantageously 2 to 3 kinds, of genes may be incorporated into a single expression vector. Preference is given to the former case with the use of a retrovirus or lentivirus vector, which offer high gene transfer efficiency, and to the latter case with the use of a plasmid, adenovirus, or episomal vector and the like. Furthermore, an expression vector incorporating two kinds or more of genes and another expression vector incorporating one gene alone can be used in combination.

When a plurality of genes are incorporated in one expression vector, these genes can advantageously be inserted into the expression vector via an intervening sequence enabling polycistronic expression. By using an intervening sequence enabling polycistronic expression, it is possible to more efficiently express a plurality of genes incorporated in one kind of expression vector.

An expression vector harboring a heterologous nucleic acid sequence encoding BDR3R as a nuclear reprogramming factor can be introduced into a cell by a technique known per se according to the choice of the vector. In the case of a viral vector, for example, a plasmid containing the nucleic acid is introduced into an appropriate packaging cell (e.g., Plat-E cells) or a complementary cell line (e.g., 293-cells), the viral vector produced in the culture supernatant is recovered, and the vector is infected to the cell by a method suitable for the viral vector. For example, specific means using a retroviral vector are disclosed in WO2007/69666, Takahashi & Yamanaka (2006) Cell 126: 663-676, and Takahashi et al., (2007) Cell 131: 861-872. Specific means using a lentivirus vector is disclosed in Yu et al., (2007) Science 318: 1917-1920.

When iPS cells are utilized as a source of cells for regenerative medicine, it is advantageous that the reprogramming gene be expressed transiently, without being integrated into the chromosome of the cells because the expression (reactivation) of the reprogramming gene possibly increases the risk of carcinogenesis in the tissues regenerated from a differentiated cell from an iPS cell. From this viewpoint, use of an adenoviral vector, whose integration into chromosome is rare, is most advantageous. Because adeno-associated virus is also low in the frequency of integration into chromosome, and is lower than adenoviral vectors in terms of cytotoxicity and inflammation inducibility, it can be mentioned as another most advantageous vector. Because Sendai viral vector is capable of being stably present outside the chromosome, and can be degraded and removed using an siRNA as required, it is advantageously utilized as well. Regarding Sendai viral vector, one described in Nishimura et al., (2007) J. Biol. Chem., 282: 27383-27391, Proc. Jpn. Acad., Ser. B 85, 348-362 (2009) or JP Patent No. 3602058 can be used.

When a retroviral vector or a lentiviral vector is used, even if silencing of the transgene has occurred, it possibly becomes reactivated; therefore, for example, a method can be used advantageously wherein a nucleic acid that encodes a nuclear reprogramming factor is cut out using the Cre/loxP system, when it has become unnecessary. That is, with a loxP sequence arranged on both ends of the nucleic acid in advance, iPS cells are induced, thereafter the Cre recombinase is allowed to act on the cells using a plasmid vector or adenoviral vector, and the region sandwiched by the loxP sequences can be cut out. Because the enhancer-promoter sequence of the LTR U3 region possibly upregulates a host gene in the vicinity thereof by insertion mutation, it is more advantageous to avoid the expression regulation of the endogenous gene by the LTR outside of the loxP sequence remaining in the genome without being cut out, using a 3′-self-inactivated (SIN) LTR prepared by deleting the sequence, or substituting the sequence with a polyadenylation sequence such as of SV40. Specific means using the Cre-loxP system and SIN LTR is disclosed in Chang et al., (2009) Stem Cells 27: 1042-1049).

A plasmid vector can be transferred into a cell using the lipofection method, liposome method, electroporation method, calcium phosphate co-precipitation method, DEAF dextran method, microinjection method, gene gun method and the like. Specific means using a plasmid as a vector are described in, for example, Science 322: 949-953 (2008) and the like.

When a plasmid vector or adenovirus vector or the like is used, gene transfer can be performed once or more optionally chosen times (e.g., once to 10 times, or once to 5 times). When two or more kinds of expression vectors are introduced into a somatic cell, it is advantageous that these all kinds of expression vectors be concurrently introduced into a somatic cell; however, even in this case, the transfection can be performed once or more optionally chosen times (e.g., once to 10 times, once to 5 times or the like), advantageously the transfection can be repeatedly performed twice or more (e.g., 3 times or 4 times).

Also when an adenovirus or a plasmid is used, the transgene can get integrated into chromosome; therefore, it is eventually necessary to confirm the absence of insertion of the gene into chromosome by Southern blotting or PCR. For this reason, like the aforementioned Cre-loxP system, it can be advantageous to use a means wherein the transgene is integrated into a chromosome, thereafter the gene is removed. In another most advantageous mode of embodiment, a method can be used wherein the transgene is integrated into chromosome using a transposon, thereafter a transposase is allowed to act on the cell using a plasmid vector or adenoviral vector so as to completely eliminate the transgene from the chromosome. As examples of advantageous transposons, piggyBac, a transposon derived from a lepidopterous insect, and the like can be mentioned. Specific means using the piggyBac transposon are disclosed in Kaji et al., (2009) Nature 458: 771-775 (2009); Woltjen et al., (2009) Nature 458: 766-770.

Another most advantageous non-recombination type vector is an episomal vector autonomously replicable outside the chromosome. A specific procedure for using an episomal vector is disclosed by Yu et al. in Science 324, 797-801. As required, an expression vector may be constructed by inserting a reprogramming gene into an episomal vector having loxP sequences placed in the same orientation at both the 5′ and 3′ sides of the vector element essential for the replication of the episomal vector, and this may be transferred into a somatic cell. Examples of the episomal vector include vectors comprising a sequence required for its autonomous replication, derived from EBV, SV40 and the like, as a vector element. Specifically, the vector element required for its autonomous replication is a replication origin or a gene that encodes a protein that binds to the replication origin to regulate its replication. Examples include the replication origin oriP and EBNA-1 gene for EBV, and the replication origin on and SV40 large T antigen gene for SV40.

The episomal expression vector contains a promoter that controls the transcription of the reprogramming gene. The promoter used can be the same promoter as the above. The episomal expression vector may further comprise an enhancer, poly-A addition signal, selection marker gene and the like as desired. Examples of selection marker gene include the dihydrofolate reductase gene, neomycin resistance gene and the like.

An episomal vector can be introduced into a cell using, for example, lipofection method, liposome method, electroporation method, calcium phosphate co-precipitation method, DEAE dextran method, microinjection method, gene gun method and the like. Specifically, the method described in Science 324: 797-801 (2009), for example, can be used.

When a nuclear reprogramming factor capable of inducing iPS cell by combination with BRD3R is a low-molecular compound, introducing thereof into a somatic cell can be achieved by dissolving the substance at an appropriate concentration in an aqueous or non-aqueous solvent, adding the solution to a medium suitable for cultivation of somatic cells isolated from human or mouse (e.g., minimal essential medium (MEM) comprising about 5 to 20% fetal bovine serum, Dulbecco's modified Eagle medium (DMEM), RPMI1640 medium, 199 medium, F12 medium and combinations thereof, and the like) so that the nuclear reprogramming factor concentration will fall in a range that is sufficient to cause nuclear reprogramming in somatic cells and does not cause cytotoxicity, and culturing the cells for a given period. The nuclear reprogramming factor concentration varies depending on the kind of nuclear reprogramming factor used, and is chosen as appropriate over the range of about 0.1 nM to about 100 nM. Duration of contact is not particularly limited, as far as it is sufficient to cause nuclear reprogramming of the cells; usually, the nuclear reprogramming factor may be allowed to be co-present in the medium until a positive colony emerges.
iPS Cell Establishment Efficiency Improvement by BRD3R: In recent years, a wide variety of substances that improve the efficiency of establishment of iPS cells, which has traditionally been low, have been proposed one after another. The efficiency of establishment of iPS cell can be expected to be increased by bringing these iPS cell establishment efficiency improvers into contact with a somatic cell.

Examples of iPS cell establishment efficiency improvers include, but are not limited to, histone deacetylase (HDAC) inhibitors (e.g., valproic acid (VPA) (Nat. Biotechnol., 26: 795-797 (2008)), low-molecular inhibitors such as trichostatin A, sodium butyrate, MC 1293, and M344, nucleic acid-based expression inhibitors such as siRNAs and shRNAs against HDAC (e.g., HDAC1 siRNA Smartpool® (Millipore), HuSH 29mer shRNA Constructs against HDAC1 (OriGene)), and the like], DNA methyltransferase inhibitors (e.g., 5′-azacytidine) (Nat. Biotechnol., 26: 795-797 (2008)), G9a histone methyltransferase inhibitors (e.g., low-molecular inhibitors such as BIX-01294 (Cell Stem Cell, 2: 525-528 (2008), nucleic acid-based expression inhibitors such as siRNAs and shRNAs against G9a (e.g., G9a siRNA (human) (Santa Cruz Biotechnology) and the like) and the like], L-channel calcium agonists (e.g., Bayk8644) (Cell Stem Cell 3: 568-574 (2008)), p53 inhibitors (e.g., siRNA and shRNA against p53, UTF1, Wnt Signaling inducers (e.g., soluble Wnt3a) (as described in Cell Stem Cell 3: 132-135 (2008)), 2i/LIF, ES cell-specific miRNAs (e.g., miR-302-367 cluster (Mol. Cell. Biol. doi:10.1128/MCB.00398-08), miR-302 (RNA (2008) 14: 1-10), miR-291-3p, miR-294 and miR-295 (described in Nat. Biotechnol. 27: 459-461 (2009))) and the like. The present disclosure provides a novel iPS cell establishment efficiency improver, BRD3R that may be used in conjunction with a nuclear reprogramming factor such as, but not limited to, NANOG and LIN28 to induce the formation of iPSCs.

Contact of an iPS cell establishment efficiency improver with a somatic cell can be achieved as described above for each of cases: (a) the improver is a proteinaceous factor, (b) the improver is a nucleic acid that encodes the proteinaceous factor, and (c) the improver is a low-molecular compound.

An iPS cell establishment efficiency improver may be brought into contact with a somatic cell simultaneously with a nuclear reprogramming factor, or either one may be contacted in advance, as far as the efficiency of establishment of iPS cells from the somatic cell is significantly improved, compared with the absence of the improver. For example, when the nuclear reprogramming factor is a nucleic acid that encodes a proteinaceous factor and the iPS cell establishment efficiency improver is a chemical inhibitor, the iPS cell establishment efficiency improver can be added to the medium after the cell is cultured for a given length of time after the gene transfer treatment, because the nuclear reprogramming factor involves a given length of time lag from the gene transfer treatment to the mass-expression of the proteinaceous factor, whereas the iPS cell establishment efficiency improver is capable of rapidly acting on the cell. When a nuclear reprogramming factor and an iPS cell establishment efficiency improver are both used in the form of a viral or non-viral vector, for example, both may be simultaneously introduced into the cell.

After the nuclear reprogramming factor(s) (and iPS cell establishment efficiency improver(s)) is (are) brought into contact with the cell, the cell can be cultured under conditions suitable for the cultivation of, for example, ES cells. In the case of mouse cells, the cultivation is carried out with the addition of Leukemia Inhibitory Factor (LIF) as a differentiation suppressor to an ordinary medium. Meanwhile, in the case of human cells, it is desirable that basic fibroblast growth factor (bFGF) and/or stem cell factor (SCF) be added in place of LIF. Usually, the cells are cultured in the co-presence of mouse embryo-derived fibroblasts (MEFs) treated with radiation or an antibiotic to terminate the cell division thereof, as feeder cells. Usually, STO cells and the like are commonly used as MEFs, but for inducing iPS cells, SNL cells (McMahon & Bradley (1990) Cell 62: 1073-1085) and the like are commonly used. Co-culture with feeder cells may be started before contact of the nuclear reprogramming factor, at the time of the contact, or after the contact (e.g., 1-10 days later).

A candidate colony of iPS cells can be selected by a method with drug resistance and reporter activity as indicators, and also by a method based on visual examination of morphology. As an example of the former, a colony positive for drug resistance and/or reporter activity is selected using a recombinant somatic cell wherein a drug resistance gene and/or a reporter gene is targeted to the locus of a gene highly expressed specifically in pluripotent cells (e.g., Fbx15, Nanog, Oct3/4 and the like, advantageously Nanog or Oct3/4). Examples of such recombinant somatic cells include MEFs from a mouse having a gene encoding a fusion protein of β-galactosidase and neomycin phosphotransferase knocked-in to the Fbx15 locus (Takahashi & Yamanaka 2006) Cell 126: 663-676), MEFs from a transgenic mouse having the green fluorescent protein (GFP) gene and the puromycin resistance gene integrated in the Nanog locus (Okita et al., (2007) Nature 448: 313-317) and the like. Although the method using reporter cells is convenient and efficient, it is desirable from the viewpoint of safety that colonies be selected by visual examination when iPS cells are prepared for the purpose of human treatment.

The identity of the cells of a selected colony as iPS cells can be confirmed by positive responses to the above-described Nanog (or Oct3/4) reporters (puromycin resistance, GFP positivity and the like), as well as by the formation of a visible ES cell-like colony; however, to increase the accuracy, it is possible to perform tests such as alkaline phosphatase staining, analysis of the expression of various ES-cell-specific genes, and transplantation of the selected cells to a mouse and confirmation of teratoma formation.

The iPS cells thus established can serve various purposes. For example, differentiation of the iPS cells into a wide variety of cells (e.g., myocardial cells, blood cells, nerve cells, vascular endothelial cells, insulin-secreting cells and the like) can be induced by means of a reported method of differentiation induction of ES cells. Therefore, inducing iPS cells using somatic cells collected from a patient or another person of the same or substantially the same HLA type would enable stem cell therapy based on transplantation, wherein the iPS cells are differentiated into desired cells (cells of an affected organ of the patient, cells having a therapeutic effect on disease, and the like), and the differentiated cells are transplanted to the patient. Furthermore, because functional cells (e.g., liver cells) differentiated from iPS cells are thought to better reflect the actual state of the functional cells in vivo than do corresponding existing cell lines, they can also be suitably used for in vitro screening for the effectiveness and toxicity of pharmaceutical candidate compounds and the like.

One aspect of the disclosure, therefore, encompasses embodiments of a method of generating an induced pluripotent stem cell (iPSC), said method comprising the steps of: introducing to an animal somatic cell at least one nuclear reprogramming inducing factor and a BRD3R polypeptide having an amino acid sequence having at least 90% sequence similarity to the amino acid sequence according to SEQ ID NO: 47, or at least one nucleic acid expressing said at least one nuclear reprogramming factor and said BRD3R-related polypeptide in the recipient somatic cell, and generating a population of induced pluripotent stem cells (iPSCs) by culturing the recipient somatic cell under conditions that promote the proliferation of said cell.

In some embodiments of this aspect of the disclosure the amino acid sequence can have at least 90% sequence similarity to the amino acid sequence according to SEQ ID NO: 47 and can be expressed from a recombinant expression vector comprising a nucleotide sequence encoding said amino acid sequence operably linked to a gene expression promoter

In some embodiments of this aspect of the disclosure the expression vector can be a lentivirus expression vector.

In some embodiments of this aspect of the disclosure the at least one nucleic acid expressing said at least one nuclear reprogramming factor can be inserted in a recombinant expression vector. In some embodiments the disclosure the expression vector is a lentivirus expression vector.

In embodiments of this aspect of the disclosure the introduction of said BRD3R-related polypeptide into the recipient somatic cell can increase the efficiency of inducing the generation of an iPSC by the at least one nuclear reprogramming inducing factor compared to when said BRD3R-related polypeptide is not introduced into the recipient somatic cell.

In some embodiments of this aspect of the disclosure the nuclear reprogramming inducing factor or a combination of said factors can be selected from the group consisting of: (1) OCT4, or a nucleic acid sequence that encodes the same; (2) SOX2, or a nucleic acid sequence that encodes the same; (3) KLF4, or a nucleic acid sequence that encodes the same; (4) OCT4 and SOX2, or nucleic acid sequences that encode the same; (5) OCT4 and KLF4, or nucleic acid sequences that encode the same; (6) SOX2 and KLF4, or nucleic acid sequences that encode the same; (7) OCT4, SOX2 and KLF4, or nucleic acid sequences that encode the same.

In some embodiments of this aspect of the disclosure the combination of nuclear reprogramming inducing factors of (4)-(7) can be expressed from a single nucleic acid sequence or individual nucleic acid sequences.

Another aspect of the disclosure encompasses embodiments of an expression vector comprising a nucleotide sequence encoding a polypeptide having an amino acid sequence having at least 90% sequence similarity to the amino acid sequence according to SEQ ID NO: 47, wherein said nucleotide sequence is operatively linked to a region of the expression vector that provides expression of the nucleotide sequence in a recipient cell.

In some embodiments of this aspect of the disclosure the expression vector further comprising at least one nucleic acid region encoding a nuclear reprogramming inducing factor or a combination of said factors, wherein said nucleotide sequence is operatively linked to a region of the expression vector that provides expression of the nucleotide sequence in a recipient cell.

In some embodiments of this aspect of the disclosure the nuclear reprogramming inducing factor or a combination of said factors can be selected from the group consisting of: (1) OCT4, or a nucleic acid sequence that encodes the same; (2) SOX2, or a nucleic acid sequence that encodes the same; (3) KLF4, or a nucleic acid sequence that encodes the same; (4) OCT4 and SOX2, or nucleic acid sequences that encode the same; (5) OCT4 and KLF4, or nucleic acid sequences that encode the same; (6) SOX2 and KLF4, or nucleic acid sequences that encode the same; (7) OCT4, SOX2 and KLF4.

In some embodiments of this aspect of the disclosure the expression vector is a lentivirus expression vector.

Another aspect of the disclosure encompasses embodiments of a modified animal somatic cell, wherein said cell can comprise a polypeptide having an amino acid sequence having at least 90% sequence similarity to the polypeptide BRD3R, or a heterologous nucleic acid expressing said BRD3R-related polypeptide.

In some embodiments of this aspect of the disclosure the modified animal somatic cell can be genetically modified by a heterologous nucleic acid expressing the BRD3R-related polypeptide.

In some embodiments of this aspect of the disclosure the modified animal somatic cell can be further modified by a heterologous nucleic acid expressing a nuclear reprogramming inducing factor or a combination of said factors selected from the group consisting of: (1) OCT4, or a nucleic acid sequence that encodes the same; (2) SOX2, or a nucleic acid sequence that encodes the same; (3) KLF4, or a nucleic acid sequence that encodes the same; (4) OCT4 and SOX2, or nucleic acid sequences that encode the same; (5) OCT4 and KLF4, or nucleic acid sequences that encode the same; (6) SOX2 and KLF4, or nucleic acid sequences that encode the same; (7) OCT4, SOX2 and KLF4, or nucleic acid sequences that encode the same.

In some embodiments of this aspect of the disclosure the combination of nuclear reprogramming inducing factors of (4)-(7) can be expressed from a single nucleic acid sequence or individual nucleic acid sequences.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include ±1%, ±2%, ±3%, ±4%, ±5%, ±6%, ±7%, ±8%, ±9%, or ±10%, or more of the numerical value(s) being modified. In addition, the phrase “about ‘x’ to ‘y’” includes “about ‘x’ to about ‘y’”.

It should be emphasized that the above-described embodiments of the present disclosure are merely possible examples of implementations, and are set forth only for a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

### EXAMPLES

### Example 1

Modification of lentiviral reprogramming constructs: The lentiviral vector pLVX-AcGFP-C1 (Clontech, 632155) was modified to generate lentiviral vector pLVH-EF1α-GFP-P2A (FIG. 6A) for more sensitive screen of cDNA library in search of new reprogramming factors. The modifications include: 1) replacement of a CMV promoter with an EF1α promoter because the CMV promoter is silenced prematurely during reprogramming; 2) removal of PGK promoter and puromycin resistant gene to reduce the size of vector for enhanced packaging; 3) realization of GFP co-expression with the reprogramming factor via the short and efficient P2A self-cleavage peptide.

### Example 2

Cloning of Gateway lentiviral destination vector and preparation of a lentiviral human kinase library: Clontech lentiviral vector was modified to construct a Gateway lentiviral destination vector for cDNA library construction (pLVH-EF1α-DEST) (FIG. 6J), as was generated vector pLVH-EF1α-GFP-P2A except that GFP was removed to reduce the size of the plasmid and for easy cloning of kinase cDNAs, and a cassette encompassing Gateway cloning sites was cloned immediately after the EF1α promoter from the destination vector pLX304 (Addgene, 25890). 89 of the human kinase cDNAs (Addgene, Human Kinase ORF kit, 1000000014) were then transferred into the lentiviral vector pLVH-EF1α-DEST using Gateway cloning kit (Life Technologies, 11791-043) per manufacturer's instruction.

### Example 3

Optimization of screening protocol: Several strategies to make the screening of cDNA library more efficient and sensitive were used. First, the lentiviral reprogramming vector was modified so that it is more efficient and consistent in reprogramming human cells. This was achieved by using EF1a promoter and co-expression of GFP, which makes titration of viral vectors easier and faster. Second, the efficient Gateway cloning was used to transfer the human kinase library onto the modified lentiviral destination vector. 24 randomly selected of the 89 cloned cDNAs were sequenced, and verified precise cloning for all of the 24 genes. Third, a protocol to simultaneously package 24×n individual transgene viruses in individual wells of 6-well plates was established. The kinase virus was not concentrated and the supernatant directly used in screening protocol. Almost 100% of transduction of BJ cells in one well of a 24-well plate with 250 μl of supernatant using GFP reporter construct on the same destination vector (FIGS. 6J and 6K) was achieved. Two cDNAs (PION and CAMKK1) from the library were also randomly tested, and demonstrated that both genes are efficiently overexpressed with viruses packaged with the protocol using cDNA plasmid cloned by Gateway technology (FIG. 6H). Fourth, reprogramming in one well of a 24-well plate was initiated to evaluate the reprogramming activities of 22×n cDNAs at one time (the two remaining wells are used for control reprogramming). Fifth, a feeder-free/serum-free reprogramming system was used. This system was reported to have high efficiency of reprogramming, and is more consistent since it is a chemically defined system (without the variation of serum and feeder) (Chen et al., (2011) Nat. Methods 8: 424-429). Last, MYC was omitted from the screening reprogramming, considering that MYC is not an essential reprogramming factor, and was reported to be detrimental in serum-free reprogramming system (Xu et al., (2013) J. Biol. Chem. 288: 9767-9778). A slight decrease in reprogramming efficiency was seen when MYC was included in the reprogramming system (FIGS. 1D and 1E). With the above improvement, an efficient and sensitive reprogramming protocol was established for evaluation of at least 22 genes at one time. To test the sensitivity of the new screening protocols, the reprogramming activities of two established reprogramming factors: NANOG and LIN28, were evaluated. The protocol of the disclosure revealed a 5.1× increase by NANOG, and a 2.4× increase by LIN28 in reprogramming efficiency (FIGS. 6I and 6J). These results are in agreement with literature that NANOG and LIN28 are relatively weak reprogramming factors. Therefore, the new screening protocol of the disclosure is sensitive and suitable for evaluation of many genes simultaneously.

Briefly (FIG. 1A), 2×10⁴ of BJ cells were seeded in each well of a 24-well plates. The second day, fibroblasts were transduced with OCT4 (10 MOI), SOX2 (5 MOI), KLF4 (5 MOI) along with 250 μl of individual kinase viral supernatant freshly packaged in one well of a 6-well plate. Twenty-two cDNAs were evaluated in one 24-well plate. One well is OSK control, and one well of cells is transduced with OSK plus 250 μl of GFP viral supernatant as a second control. Virus was removed next day with fresh fibroblast medium. Forty-eight hours after transduction, fibroblasts were transferred from one well into a 60-mm dish for continued reprogramming. The next day of re-seeding, fibroblast medium was replaced with E7 medium (E8 minus TGF beta) plus 100 μM of sodium butyrate. From day 18 of reprogramming on, E8 media was used. On day 25 of reprogramming, reprogramming dishes were stained for ALP or TRA-1-60 markers.

### Example 4

Package kinase viruses in one well of a 6-well plate: Six-well plate was coated with collagen I (5 μg/cm², BD Bioscience, 354236). The day before transfection, lenti-X 293T (Clontech, 632180) were seeded at 6×10⁵ cells/well, and the cells were cultured in 2 ml of DMEM (Gibco, 12800-058) with 10% FBS (Gibco, 10437 or 26140), 4 mM L-Glutamine; 100 U/ml penicillin and 100 μg/ml streptomycin (Gibco, 15140-122), 0.1 mM MEM NEAA (Gibco, 11140-050). At 24 hours, the medium was replaced 1-3 h prior to transfection with 1.6 ml of pre-warmed fresh medium. A total of 4 μg of plasmid DNA (0.7 μg envelope plasmid (pMD2-G) was added, 1.3 μg packaging plasmid (ps-PAX2) and 2 μg transfer plasmid) was added to 100 μl of 0.25 M calcium chloride solution. The diluted plasmid DNA was mixed with an equal volume of 2× HBS (100 μl) (PH 7.07) and mixed by pipetting 10-20 times gently using a 200-ul pipette. 200 μl of the DNA complex was added to a well drop-wise, and the plate gently swirled. The cells were incubated for 12-18 h, the DNA and spent media removed at 12-18 h after DNA addition, and 1.6 ml of fresh DMEM added to the each well before incubation at 37° C., 5% CO₂. Forty-eight to 72 hours after the medium change, virus-containing supernatant was collected and filtered using 0.45-μm filters.

### Example 5

Concentration of virus: The reprogramming viruses (OCT4, SOX2, KLF4, MYC and BET members) were concentrated before use except for library viruses. The lentiviral supernatant was centrifuged at 3,000×g for 10 mins at 4° C. to remove the cell debris. Thirty ml of the viral supernatant was then transferred into each 50-ml tube. 7 ml of 50% PEG-6000 stock solution (final concentration of 8.5%) and 4.1 ml of 4 M NaCl stock solution was added to each tube (final concentration of 0.4 M). The virus mixture was stored at 4° C. for 3-5 h.

The contents were mixed every 20-30 min and the viruses centrifuged at 4,000×g for 30 min at 4° C. Carefully decant the supernatant and add Tris-HCl buffer (50 mM, pH 7.4) at 1/100 to 1/150 of the volume of the original viral supernatant. The pellets were resuspended aliquoted. The concentrated virus was stored at −80° C. The virus was titrated with flow cytometry based on GFP expression in Hela cells transduced with viral stock.

### Example 6

Cell culture and reprogramming: Human fibroblast cells (BJ ATCC CRL-2522®) were cultured in fibroblast medium: DMEM, 10% heat-inactivated FBS, 0.1 mM 2-mercaptoethanol, 100 U/ml penicillin, 100 μg/ml streptomycin, 0.1 mM MEM NEAA and 4 ng/ml human bFGF. For reprogramming, seed BJ cells into 24-well plate at 2×10⁴ cells/well. Twenty-four hours after plating, pre-mix the OSK (OCT4, 10 MOI; SOX2, 5 MOI and KLF4, 5 MOI) viruses (shown in FIGS. 6B-6D, SEQ ID NO: 58-60, respectively), and add the OSK virus along with 250 μl supernatant of individual kinase virus into respective wells. Incubate overnight. Next morning, remove viruses by replacing virus-containing medium with fresh fibroblast medium. Twenty-four hours after transduction, the cells were re-seeded from one well into one matrigel-coated 60-mm dish. Next day, replace fibroblast medium with reprogramming media (E7 plus sodium butyrate at 100 μM). On 18 day, start to use E8 media. On day 25 of reprogramming, stain the reprogramming cells for alkaline phosphatase or TRA-1-60.

Human ESCs (H1 and H9, WiCell, Wisconsin) and iPSCs were maintained in E8 medium (Chen et al., (2011) Nat. Methods 8: 424-429) on Matrigel-coated tissue culture vessels. E8 medium contained DMEM/F12, 64 mg/L L-ascorbic acid 2-phosphate sesquimagnesium, 13.6 μg/L sodium selenium, 1.7 g/L NaHCO₃, 1 g/L sodium chloride, 10 ng/ml FGF2, 20 μg/ml insulin, 10 μg/ml transferrin and 2 μg/L TGFβ1.

### Example 7

Immunocytochemistry and microscopy: Cells were fixed with 4% paraformaldehyde in PBS at room temperature for 15 min. The fixed cells were then blocked with 0.1% Triton X-100, 1% BSA in PBS at room temperature for 30 min. Wash 3 times with PBS; Cells were incubated with the diluted primary antibody overnight at 4° C. Cell was washed 3 times and then incubated with appropriate secondary antibody at room temperature in the dark for one h. After washing the cells with PBS add DAPI (2 μg/ml) and incubate at room temperature for 5-10 min. For immunocytochemistry in confocal imaging, following the same procedure above except for that cells were cultured on fibronectin-coated coverslips (NeuVitro, GG-14-fibronectin). Fluorescence microscopy was performed on Olympus IX51 equipped with CellSens software for image acquisition. Confocal images were acquired on a Nikon A1 Laser Confocal system with a Nikon Eclipse Ti microscope, which has a 60× Plan Apo objective. Lasers used were 405 nm for blue, 488 nm for green, 561 nm for red. NIS Elements 4.20.01 Software was used to acquire Z-stacks of each channel sequentially to avoid spectral cross talk. Each slice was captured at 0.4-μm step, and reconstructions were done with a Maximum Intensity Projection and a 3D Rendered Maximum Projection.

### Example 8

Western blotting: Total cell lysates were prepared by incubating cells in RIPA buffer (100 mM Tris-HCl pH 7.4, NaCl 150 mM, EDTA 1 mM, 1% TritonX-100, 1% sodium deoxycholate and 0.1% SDS) on a rotator for 1 h at 4° C. Centrifuge at 13,000×g for 10 min. Proteins were resolved on 10% SDS-polyacrylamide gel, and the proteins were transferred to polyvinylidene difluoride membranes (Bio-Rad, 1620177). Membranes were blocked with 5% milk in Tris-buffered Saline with Tween 20 (TBST) for at least 1 h at RT. Blots were then probed with the antibodies.

### Example 10

shRNA cloning: BRD3R shRNAs at XbaI and HpaI sites on the shRNA vector PLVH-U6-EF1a-AcGFP were cloned. Correct cloning was verified by sequencing. An shRNA targeting the firefly luciferase was used as a control. The oligonucleotide primers for the cloning of BRD3R shRNA (SEQ ID NO: 42 and 43) are listed in Table 3.

### Example 9

RT-qPCR: Cells were harvested with Trizol reagent and stored at −80° C. until use. Total RNA was extracted using the Direct-zol™ Miniprep kit (R2052). cDNA was prepared using the M-MLV reverse transcriptase (cat #28025-013) per manufacturer's instruction. Quantitative PCR was performed on ViiA 7 Real-time PCR system (Applied Biosystem) using SYBR-Green Master PCR mix (Clontech, Cat #639676) in triplicates. All quantifications were normalized to an endogenous GAPDH control. Primers used are listed in Table 3.

TABLE 3
PCR Primers

| primer name | sequence | Gene name | Accession # | application |
|---|---|---|---|---|
| hAURKB-F2 | AAGGAGCTGCAGAAGAGCT | AURKB | NM_001284526 | qPCR |
| | G (SEQ ID NO: 1) | | | |
| hAURKB-R2 | CCTTGAGCCCTAAGAGCAG | | | |
| | A (SEQ ID NO: 2) | | | |
| hCDK1-F1 | CTGGGGTCAGCTCGTTACT | CDK1 | NM_001786. | qPCR |
| | C (SEQ ID NO: 3) | | | |
| hCDK1-R1 | TCTGAATCCCCATGGAAAA | | | |
| | G (SEQ ID NO: 4) | | | |
| hCKS2-F1 | CACTACGAGTACCGGCATG | CKS2 | NM_001827 | qPCR |
| | TT (SEQ ID NO: 5) | | | |
| hCKS2-R1 | TGTTGGACACCAAGTCTCC | | | |
| | TC (SEQ ID NO: 6) | | | |
| hCLSPN-F2 | AAGGAGCGAATTGAACGAG | CLSPN | NM_022111 | qPCR |
| | A (SEQ ID NO: 7) | | | |
| hCLSPN-R2 | TGCAGTGCTTTGGCTGTAA | | | |
| | C (SEQ ID NO: 8) | | | |
| hDLGAP5-F2 | CGTCCAGACCGAGTGTTCT | DLGAP5 | NM_014750. | qPCR |
| | T (SEQ ID NO: 9) | | | |
| hDLGAP5-R2 | ATCCTTCCTGTGTCGACTG | | | |
| | G (SEQ ID NO: 10) | | | |
| hFAM83D-F1 | CAGTGGTCATGGACGTGTT | FAM83D | NM_030919. | qPCR |
| | C (SEQ ID NO: 11) | | | |
| hFAM83D-R1 | CAACTCCCTGTTTCCTGCAT | | | |
| | (SEQ ID NO: 12) | | | |
| hNCAPH-F2 | GGCTCAGAACCTCTCCATA | NCAPH | NM_001281710 | qPCR |
| | CCT (SEQ ID NO: 13) | | | |
| hNCAPH-R2 | GAGGTCCTCTGTTCCTTCC | | | |
| | AGT (SEQ ID NO: 14) | | | |
| hNUSAP1-F2 | AAGCGCTCTGCTATCTCTG | NUSAP1 | NM_016359 | qPCR |
| | C (SEQ ID NO: 15) | | | |
| hNUSAP1-R2 | TTCTGGCTGGAGTCTTGGT | | | |
| | C (SEQ ID NO: 16) | | | |
| hSPC25-F2 | TTCAAAAGTACGGACACCT | SPC25 | NM_020675 | qPCR |
| | CCT (SEQ ID NO: 17) | | | |
| hSPC25-R2 | CTCAACCATTCGTTCTTCTT | | | |
| | CC (SEQ ID NO: 18) | | | |
| hTACC3-F2 | TTTCGCCACCAGAAGTTAC | TACC3 | NM_006342 | qPCR |
| | C (SEQ ID NO: 19) | | | |
| hTACC3-R2 | TCATAGCTTTGGCCAGGTT | | | |
| | C (SEQ ID NO: 20) | | | |
| hUBE2C-F1 | ACCCAACATTGATAGTCCCT | UBE2C | NM_007019 | qPCR |
| | TG (SEQ ID NO: 21) | | | |
| hUBE2C-R1 | GCTGGTGACCTGCTTTGAG | | | |
| | TAG (SEQ ID NO: 22) | | | |
| 3R1F | GCAGAGATCATTTCTTGAC | BRD3R | BC032124.2 | qPCR |
| | CTGTGGAG (SEQ ID NO: 23) | | | |
| 3R1R | AGCCCTTGGCCAGGAAACA | | | |
| | A (SEQ ID NO: 24) | | | |
| 3LF | CTTCAAATGCTAACCCGAT | BRD3 | NM_007371.3 | qPCR |
| | GAC (SEQ ID NO: 25) | | | |
| 3LR | TCTTTCTCGAGCTATCGACC | | | |
| | AG (SEQ ID NO: 26) | | | |
| BRD3iso2HA- | GTTCCAGATTACGCTATGTC | BRD3R | BC032124.2 | cloning |
| F | CACCGCCACGACA (SEQ ID | | | |
| | NO: 27) | | | |
| BRD3iso2HA- | ATCGTATGGGTACATAGCC | | | |
| R | TGCTTTTTTGTACAAACTTG | | | |
| | (SEQ ID NO: 28) | | | |
| BRD2-F | ATGCTGCAAAACGTGACTC | BRD2 | NM_001113182.2 | cloning |
| | CCCACA (SEQ ID NO: 29) | | | |
| BRD2-R | TTAGCCTGAGTCTGAATCA | | | |
| | CTGGTGTC (SEQ ID NO: 30) | | | |
| 3DF | ATGTCCACCGCCACGACAG | BRD3 | NM_007371.3 | cloning |
| | TCGC (SEQ ID NO: 31) | | | |
| 3DR | TCATTCTGAGTCACTGCTGT | | | |
| | CAGAGCT (SEQ ID NO: 32) | | | |
| hpion-1-F | TCTCTGCCTGCCATTCATTT | PION | NM_017439.3 | qPCR |
| | (SEQ ID NO: 33) | | | |
| hpion-1-R | GCACTGAGGAATGTGGCAA | | | |
| | T (SEQ ID NO: 34) | | | |
| hCAMKK1-1- | GCGTCAGCAACCAGTTTGA | CAMKK1 | NM_172207.2 | qPCR |
| F | G (SEQ ID NO: 35) | | | |
| hCAMKK1-1- | AGTGGCCCATACATCCAAG | | | |
| R | G (SEQ ID NO: 36) | | | |
| hm- | CCTTCATTGACCTCAACTAC | GAPDH | NM_001256799.1 | qPCR |
| GAPDH- | ATGG (SEQ ID NO: 37) | | | |
| hao-F | | | | |
| hm-GAPDH- | TCGCTCCTGGAAGATGGTG | | | |
| hao-R | ATGGG (SEQ ID NO: 38) | | | |
| attR1-F | CAACAAGTTTGTACAAAAAA | | | cloning |
| | GCTGAACG (SEQ ID NO: 39) | | | |
| attrR2-stop-R | TCAACTAGTTACTAAACCAC | | | cloning |
| | TTTGTACAAGAAAGCTGAAC | | | |
| | GAGA (SEQ ID NO: 40) | | | |
| 3R2R | TCAAACTCCACAGGTCAAG | BRD3R | BC032124.2 | cloning/PCR |
| | AAATGATC (SEQ ID NO: 41) | | | |
| BRD3S-sh3sn | CTAGGAACCTCTGTAATTG | BRD3R | BC032124.2 | Cloning |
| | TTTCCTGGCTCGAGCCAGG | | | shRNA |
| | AAACAATTACAGAGGTTCT | | | |
| | TTTTT (SEQ ID NO: 42) | | | |
| BRD3S-sh3as | AAAAAAGAACCTCTGTAATT | | | |
| | GTTTCCTGGCTCGAGCCAG | | | |
| | GAAACAATTACAGAGGTTC | | | |
| | (SEQ ID NO: 43) | | | |

### Example 10

shRNA cloning: BRD3R shRNAs at XbaI and HpaI sites on the shRNA vector PLVH-U6-EF1a-AcGFP were cloned. Correct cloning was verified by sequencing. An shRNA targeting the firefly luciferase was used as a control. The oligonucleotide primers for the cloning of BRD3R shRNA are SEQ ID NO: 42 and 43 as listed in Table 3.

### Example 11

Mitotic shake-off: Reprogramming cells were prepared in in T75 flasks. On day 4 of reprogramming, 1 h before mitotic shake-off, replace spent media with fresh reprogramming media. Shake the flasks at 200 rpm for 1 min and collect the media containing the shake-off mitotic cells. Add new warmed media and incubate for 10 min. Repeat these shake-off collection 2 more times. Pool the cells and centrifuge at 1,000×g for 5 min. Count the cells collected.

### Example 12

Cell proliferation assays: Human fibroblasts were transduced with reprogramming viruses. Forty-eight hs post transduction, the reprogramming cells were plated at 4,000 cells/well of a 96-well plate. Five replicates were performed for each condition. On days 0, 1, 3, 5, 7, 9, 11 and 13, the cells were measured using a CyQUANT® NF Cell Proliferation Assay Kit (Life Technologies; c35007) per manufacturer's instruction.

### Example 13

Cell Cycle Analysis: Harvest cells by trypsin solution. Fix cell with 70% cold ethanol overnight at 4° C. The next day, wash cells with PBS. Treat cells with 0.2 mg/ml RNase A in PBS containing 0.1% Triton X-100 at 37° C. for 1 h. Add PI at a final concentration of 10 μg/ml. Keep the cells in dark and at 4° C. until analysis. Analyze on BD LSRFortessa. Percentage of cells at each cell cycle phases was determined with Watson (pragmatic) (Watson et al., (1987) Cytometry 8: 1-8) and Dean-Jett-Fox (Fox M. H. (1980) Cytometry 1: 71-77) models on FlowJow.

### Example 14

Senescence analysis: Prepare reprogramming cells as stated in the reprogramming section. At day 5 of reprogramming, stain cells for endogenous p-galactosidase using the Cell Senescence Kit (Cell Signaling Technology, #9860s) per manufacturer's instruction. Count the β-galactosidase⁺ cells in 10 randomly selected fields in each treated groups. The total cells were counted based on DAPI staining.

### Example 15

EB generation and in vitro differentiation of iPSCs: EB was generated from established iPSCs using AggreWell® 400 (Stemcell Technologies, 27845) per manufacturer's instruction. EBs (at age of day 4) were plated on gelatin-coated plates in DMEM with 10% FBS and differentiate for three weeks. Change media every two days.

### Example 16

Teratoma formation assays: The iPSC lines were cultured on matrigel-coated vessels in E8 medium. At 80% confluence, harvest cells using the EDTA. Re-suspend 10⁶ cells in 100 μl of cold E8 containing 30% Matrigel. Inject the cells subcutaneously into one flank of a mouse. After 6 to 8 weeks, harvest the teratoma and fix the teratoma in formaldehyde. Histology was performed at UAB Comparative Pathology Laboratory.

### Example 17

RNA sequencing: mRNA sequencing was performed on the Illumina HiSeq2500 using the sequencing reagents and flow cells providing up to 300 Gb of sequence information per flow cell. Briefly, the quality of the total RNA was assessed using the Agilent 2100 Bioanalyzer followed by 2 rounds of polyA+ selection and conversion to cDNA. The stranded mRNA library generation kits were used per manufacturer's instructions (Agilent, Santa Clara, Calif.). Library construction consists of random fragmentation of the polyA mRNA, followed by cDNA production using random primers with inclusion of Actinomycin D in the first strand reaction. The ends of the cDNA are repaired, polyA-tailed and adaptors ligated for indexing (4 different barcodes per lane) during the sequencing runs. The cDNA libraries were quantitated using qPCR in a Roche LightCycler 480 with the Kapa Biosystems kit for library quantitation (Kapa Biosystems, Woburn, Mass.) prior to cluster generation. Clusters were generated to yield approximately 725 K to 825 K clusters/mm². Cluster density and quality were determined during the run after the first base addition parameters were assessed. Paired end 2×50 bp sequencing runs were run to align the cDNA sequences to the reference genome.

### Example 18

Bioinformatics: 25-65 million of paired 51 bp reads were obtained for each sample. RNA sequencing reads were mapped to the human reference genome (GRCh37/hg19) using TopHat (v2.0.13) (Kim et al., (2013) Genome Biol. 14: R36). For more accurate mapping, the mean insert sizes and the standard deviations were calculated using Picard-tools (v1.126), and were passed to the mapper along with a Gene Transfer File (GTF version GRCh37.70) and the data were re-aligned. Read count tables were generated using HT-seq (v0.6.0) (Anders et al., (2015) Bioinformatics 31: 166-169). Deferential Expression (DE) analysis was performed using DESeq (v3.0) (Anders & Huber (2010) Genome Biol. 11: R106). Cufflinks v2.2.1 (Trapnell et al., (2010) Nat. Biotechnol. 28: 511-515) and Cummerbund v3.0 (Goff et al., (2014): Visualization and Exploration of Cufflinks High-throughput Sequencing Data., pp. 45) were also used for calculating expression levels in FPKM and data visualization. The BigWig files were generated using Bedtools (v2.17.0) (Quinlan & Hall (2010) Bioinformatics 26: 841-842) and bedGraphToBigWig tool (v4). For the analysis of micro-array data, Limma v3.0 (Smyth G. K., (2005) in Bioinformatics and Computational Biology Solutions Using R and Bioconductor: Gentleman et al., Eds. (Springer: New York): Ch. 23: pp. 397-420) was used Gene Ontology (GO) analysis was conducted using PANTHER (Mi et al., (2013) Nat. Protoc. 8: 1551-1566), Cytoscape-BiNGO (Saito et al., (2012) Nat. Methods 9: 1069-1076) and DAVID (Huang da et al., (2009) Nat. Protoc. 4: 44-57). Lists of mitotic genes were compiled based on the results from the three tools.

### Example 19

Histone peptide pull-down assay: H3 or H4 histone tails with 8 different acetylation modifications were evaluated for binding with BRD3R and BRD3. One unmodified tail for each histone was used for negative control. The histone tails and modifications are listed in Table 4.

TABLE 4
Histone tails used for peptide pull-down experiments

| Description of histone | Sequence of histone tails |
|---|---|
| Histone H3 N-terminal | ARTKQTARKSTGGKAPRKQLK-(Biot)-NH₂ |
| Peptide Biotinylated | (SEQ ID NO: 48) |
| Histone H3 K9ac | ARTKQTARK(Ac)STGGKAPRKQLK-(Biot)-NH₂ |
| Peptide Biotinylated | (SEQ ID NO: 49) |
| Histone H3 K14ac | ARTKQTARKSTGGK(Ac)APRKQLK-(Biot)-NH₂ |
| Peptide Biotinylated | (SEQ ID NO: 50) |
| Histone H3 K9, K14ac | ARTKQTARK(Ac)STGGK(Ac)APRKQLK-(Biot)-NH₂ |
| Peptide Biotinylated | (SEQ ID NO: 51) |
| Histone H4 N-terminal | Ac-SGRGKGGKGLGKGGAKRHRKVLR-Peg-Biot |
| Peptide Biotinylated | (SEQ ID NO: 52) |
| Histone H4 K5ac | Ac-SGRGK(Ac)GGKGLGKGGAKRHRKVLR-Peg-Biot |
| Peptide Biotinylated | (SEQ ID NO: 53) |
| Histone H4 K8ac | Ac-SGRGKGGK(Ac)GLGKGGAKRHRKVLR-Peg-Biot |
| Peptide Biotinylated | (SEQ ID NO: 54) |
| Histone H4 K12ac | Ac-SGRGKGGKGLGK(Ac)GGAKRHRKVLR-Peg-Biot |
| Peptide Biotinylated | (SEQ ID NO: 55) |
| Histone H4 K16ac | Ac-SGRGKGGKGLGKGGAK(Ac)RHRKVLR-Peg-Biot |
| Peptide Biotinylated | (SEQ ID NO: 56) |
| Histone H4 K5, K8, K12 | Ac-SGRGK(Ac)GGK(Ac)GLGK(Ac)GGAK(Ac)R |
| K16ac Peptide Biotinylated | HRKVLR-Peg-Biot (SEQ ID NO: 57) |

Human BJ fibroblasts were transduced with BRD3 or BRD3R viruses. Three days post-transduction, cells were lysed by non-denaturing lysis buffer (20 mM HEPES pH 7.9, 150 mM NaCl, 1 mM MgCl2, 0.5% NP40, 10 mM NaF, 0.2 mM NaVO4, 10 mM β-glycerol phosphate, 5% glycerol, 1 mM DTT, 1 mM PMSF and protease inhibitors). Twenty μg of the cell lysates were incubated with 1 μg biotinylated peptide in 300 μl binding buffer (50 mM Tris pH 7.5, 150 mM NaCl, 0.1% NP-40, 1 mM PMSF and protease inhibitors) at 4° C. for overnight. The next day, 30 μl of Dynabeads M-280 Streptavidin was added into each sample (Invitrogen, 11205D). The mixture of proteins, antibodies and beads were further incubated with gentle rotation at 4° C. for 1 h. The beads were then washed with binding buffer three times. The bound proteins were resuspended in 60 μl of 2× SDS sample buffer. The pull-down proteins were analyzed by western using antibody of BRD3 (Proteintech, 11859-1-AP). Two micrograms of cell lysates were loaded as input control. For semi-quantification, band density was normalized to the corresponding inputs.

1. A method of generating an induced pluripotent stem cell (iPSC), said method comprising the steps of:
introducing to an animal somatic cell at least one nuclear reprogramming inducing factor and (ii) a BRD3R polypeptide having an amino acid sequence having at least 90% sequence similarity to the amino acid sequence according to SEQ ID NO: 47, or at least one nucleic acid expressing said at least one nuclear reprogramming factor and said BRD3R-related polypeptide in the recipient somatic cell; and
generating a population of induced pluripotent stem cells (iPSCs) by culturing the recipient somatic cell under conditions that promote the proliferation of said cell.

2. The method of claim 1, wherein the amino acid sequence having at least 90% sequence similarity to the amino acid sequence according to SEQ ID NO: 47 is expressed from a recombinant expression vector comprising a nucleotide sequence encoding said amino acid sequence operably linked to a gene expression promoter.

3. The method of claim 2, wherein the expression vector is a lentivirus expression vector.

4. The method of claim 1, wherein the at least one nucleic acid encoding said at least one nuclear reprogramming factor is inserted in a recombinant expression vector and operably linked to a gene expression promoter.

5. The method of claim 4, wherein the expression vector is a lentivirus expression vector.

6. The method of claim 1, wherein the introduction of said BRD3R-related polypeptide into the recipient somatic cell increases the efficiency of inducing the generation of an iPSC by the at least one nuclear reprogramming inducing factor compared to when said BRD3R-related polypeptide is not introduced into the recipient somatic cell.

7. The method of claim 1, wherein the nuclear reprogramming inducing factor or a combination of said factors are selected from the group consisting of: (1) OCT4, or a nucleic acid sequence that encodes the same; (2) SOX2, or a nucleic acid sequence that encodes the same; (3) KLF4, or a nucleic acid sequence that encodes the same; (4) OCT4 and SOX2, or nucleic acid sequences that encode the same; (5) OCT4 and KLF4, or nucleic acid sequences that encode the same; (6) SOX2 and KLF4, or nucleic acid sequences that encode the same; (7) OCT4, SOX2 and KLF4, or nucleic acid sequences that encode the same.

8. The method of claim 7, wherein the combination of nuclear reprogramming inducing factors of (4)-(7) are expressed from a single nucleic acid sequence or individual nucleic acid sequences.

9. An expression vector comprising a nucleotide sequence encoding a polypeptide having an amino acid sequence having at least 90% sequence similarity to the amino acid sequence according to SEQ ID NO: 47, wherein said nucleotide sequence is operatively linked to a region of the expression vector that provides expression of the nucleotide sequence in a recipient cell.

10. The expression vector of claim 9, further comprising at least one nucleic acid region encoding a nuclear reprogramming inducing factor or a combination of said factors, wherein said nucleotide sequence is operatively linked to a region of the expression vector that provides expression of the nucleotide sequence in a recipient cell.

11. The expression vector of claim 10, wherein the nuclear reprogramming inducing factor or a combination of said factors are selected from the group consisting of: (1) OCT4, or a nucleic acid sequence that encodes the same; (2) SOX2, or a nucleic acid sequence that encodes the same; (3) KLF4, or a nucleic acid sequence that encodes the same; (4) OCT4 and SOX2, or nucleic acid sequences that encode the same; (5) OCT4 and KLF4, or nucleic acid sequences that encode the same; (6) SOX2 and KLF4, or nucleic acid sequences that encode the same; (7) OCT4, SOX2 and KLF4.

12. The expression vector of claim 9, wherein the expression vector is a lentivirus expression vector.

13. A modified animal somatic cell, wherein said cell comprises a polypeptide having an amino acid sequence having at least 90% sequence similarity to the polypeptide BRD3R, or a heterologous nucleic acid expressing said BRD3R-related polypeptide.

14. The modified animal somatic cell of claim 13, wherein the modified animal somatic cell is genetically modified by a heterologous nucleic acid expressing the BRD3R-related polypeptide.

15. The modified animal somatic cell of claim 14, wherein the modified animal somatic cell is further modified by a heterologous nucleic acid expressing a nuclear reprogramming inducing factor or a combination of said factors selected from the group consisting of: (1) OCT4, or a nucleic acid sequence that encodes the same; (2) SOX2, or a nucleic acid sequence that encodes the same; (3) KLF4, or a nucleic acid sequence that encodes the same; (4) OCT4 and SOX2, or nucleic acid sequences that encode the same; (5) OCT4 and KLF4, or nucleic acid sequences that encode the same; (6) SOX2 and KLF4, or nucleic acid sequences that encode the same; (7) OCT4, SOX2 and KLF4, or nucleic acid sequences that encode the same.

16. The modified animal somatic cell of claim 15, wherein the combination of nuclear reprogramming inducing factors of (4)-(7) is expressed from a single nucleic acid sequence or individual nucleic acid sequences.
